(19) United States
(12) Patent Application Publication
(10) Pub. No.: US 2010/0317607 A1
(43) Pub. Date: Dec. 16, 2010

(54) USE OF COMPOUNDS FOR PREPARING ANTI-TUBERCULOSIS AGENTS

(75) Inventors: Graham Michael Wynne, Oxfordshire (GB); Olivier De Moor, Oxford (GB); Peter David Johnson, Oxford (GB); Richard John Vickers, Oxfordshire (GB)

Correspondence Address: SEED INTELLECTUAL PROPERTY LAW GROUP PLLC, 701 FIFTH AVE, SUITE 5400, SEATTLE, WA, 98104, US

(73) Assignee: INFECTIOUS DISEASE RESEARCH INSTITUTE, Seattle, WA (US)

(21) Appl. No.: 12/666,769

(22) PCT Filed: Jun. 24, 2008
(86) PCT No.: PCT/GB08/02145

§ 371 Date: Sep. 7, 2010

(30) Foreign Application Priority Data
Jun. 27, 2007 (GB) 0712458.9
Jan. 24, 2008 (GB) 0801288.2

(51) Int. Cl. A61K 31/517; C07D 239/94; C07D 401/12; C07D 413/12; A61K 31/5377; A61K 31/7036; A61K 31/606; A61K 31/5383; A61P 31/06

(52) U.S. Cl. 514/40; 544/293; 544/284; 544/119; 514/266.4; 514/266.22; 514/234.5; 514/161; 514/230.2

(57) ABSTRACT

Compounds of a compound of compound of general formula (I) wherein X¹, X², A, R¹R², R³ and R⁴ are as defined herein; are useful as anti-mycobacterial agents, especially agents for the treatment of tuberculosis.

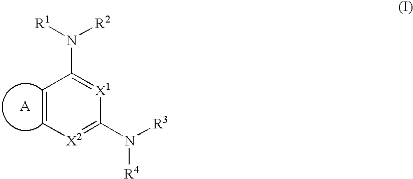

The present invention relates to compounds which are of use in the treatment of bacterial infections, to compositions containing those compounds and to methods of treating bacterial infections using the compounds. In particular, the compounds of the present invention are useful for the treatment of tuberculosis.

The development of antibacterial drugs represents one of the most important medical advances of the 20th Century. Previously untreatable diseases could now be readily controlled and it was felt that many diseases would be eradicated with these new wonder drugs. However, the emergence of drug resistant pathogens has placed many infectious diseases into the spotlight as many of the current frontline drugs are unable to effectively control many diseases.

The problem, however, is not restricted to the so-called hospital ‘superbugs’ but also encompasses diseases affecting the wider community. A particularly pertinent example is Tuberculosis (TB) which has re-emerged as a serious global health problem.

A highly contagious bacterial infection, TB has become the biggest single-infection killer in the world. Over one third of the world's population is believed to be infected, with around 5-10% of those becoming sick or infectious during their lifetime. The disease accounts for around 2 million deaths a year and is a leading cause of mortality in HIV sufferers.

TB persists in the body for months or years following infection and once the patient becomes sick, a complex and protracted treatment regime (Directly Observed Treatment Shortcourse or DOTS) of 4-5 drugs over a 6-9 month period is required to eradicate the disease. Poor patient compliance has led to a rapid increase in multi-drug resistant TB (MDR-TB). An 80% fatality rate for MDR-TB has resulted in the disease becoming a major global health problem.

TB is not a problem isolated to the developing world. With the increase in global travel and immigration TB is a serious problem for western countries. In the US alone, it is estimated that 10-15 million people are currently infected with TB and around 20,000 will become sick each year.

With only a small handful of new antibacterial classes being approved by the FDA in the last 30 years, new and innovative treatments are urgently required to address infectious diseases. Therefore, it is an object of the present invention to provide compounds for use in the treatment of mycobacterial infections and, in particular, in the treatment of tuberculosis.

Therefore, in a first aspect of the present invention, there is provided a compound of general formula (I)

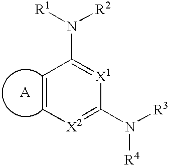

wherein

### X¹ is CH or N;

### X² is CH or N;

provided that X¹ and X² cannot both be CH;
   A is a saturated, unsaturated or partially saturated 5- or 6-membered ring system containing up to three heteroatoms chosen from N, O and S and optionally substituted with one or more substituents selected from halo, OH or C₁-C₆ alkyl, or O—(C₁-C₆ alkyl), either of which may be substituted with one or more halo substituents;
   R¹ and R³ are each independently hydrogen or C₁-C₄ alkyl optionally substituted with halo or a group R⁵;
   R² and R⁴ are each independently selected from:
   (a) a group —C₁-C₆ alkyl- or C₂-C₆ alkenyl, either of which may optionally be substituted with one or more groups NHR⁵, R⁵, R⁶, OR⁶, COR⁶, CO₂R⁶, CONR⁶R⁷;
   (b) a group R⁵ or —COR⁵;
each R⁵ is independently an aryl, heteroaryl, carbocyclic or heterocyclic group, any of which may be substituted with one or more substituents chosen from halo, CN, NO₂, R⁶, OR⁶, N(R⁶)₂, COR⁶, CO₂R⁶, SO₂R⁶, (C₁-C₆) alkyl-CO₂R⁶, (C₁-C₆) alkyl-OR⁶, NR⁷COR⁶, NR⁷CO₂R⁶, NR⁷SO₂R⁶, NR⁷CONR⁶R⁷, CONR⁶R⁷, or SO₂NR⁶R⁷;
each R⁶ is independently H, C₁-C₆ alkyl, C₂-C₆ alkenyl, C₂-C₆ alkynyl, C₄-C₇ carbocyclyl, C₄-C₇ heterocyclyl or a 5- or 6-membered aromatic or heteroaromatic ring, any of which may be substituted with one or more halo atoms; and
each R⁷ is independently hydrogen or C₁-C₄ alkyl, optionally substituted with one or more halo atoms;
   or, alternatively, R¹ and R² together with the nitrogen atom to which they are attached may form a 4- to 7-membered heterocyclic ring, optionally containing one or more further heteroatoms or groups chosen from N, O, S, SO or SO₂; and optionally fused with a 5- or 6-membered aromatic ring and optionally substituted with one or more substituents selected from R⁵ as defined above or halo, CN, NO₂, R⁶, OR⁶, N(R⁶)₂, COR⁶, CO₂R⁶, SO₂R⁶ and (C₁-C₆) alkyl-CO₂R⁶, (C₁-C₆) alkyl-OR⁶, NR⁷COR⁶, NR⁷CO₂R⁶, NR⁷SO₂R⁶, NR⁷CONR⁶R⁷, CONR⁶R⁷, or SO₂NR⁶R⁷; wherein R⁶ and R⁷ are as defined above;
   or, alternatively, R³ and R⁴ together with the nitrogen atom to which they are attached may form a 4- to 7-membered heterocyclic ring, optionally containing one or more further heteroatoms chosen from N, O, S, SO or SO₂, optionally fused with a 5- or 6-membered aromatic ring and optionally substituted with one or more substituents selected from R⁵ as defined above or halo, CN, NO₂R⁶, OR⁶, N(R⁶)₂, COR⁶, CO₂R⁶, SO₂R⁶ and (C₁-C₆) alkyl-CO₂R⁶, (C₁-C₆) alkyl-OR⁶, NR⁷COR⁶, NR⁷CO₂R⁶, NR⁷SO₂R⁶, NR⁷CONR⁶R⁷, CONR⁶R⁷, or SO₂NR⁶R⁷; wherein R⁶ and R⁷ are as defined above;
   or a pharmaceutically acceptable salt, hydrate, solvate, complex or prodrug thereof;
   for use in the treatment or prevention of a mycobacterial condition.

As used herein, the term “mycobacterial condition” defines any disease, disorder, pathology, symptom, clinical condition or syndrome in which bacteria of the genus Mycobacterium (i.e. mycobacteria) act as aetiological agents or in which infection with mycobacteria is implicated, detected or involved. The term therefore includes the various forms of tuberculosis (TB), leprosy, paediatric lymphadenitis and mycobacterial skin ulcers. The term therefore covers mycobacterial conditions arising from or associated with infection by nontuberculous mycobacteria as well as tuberculous mycobacteria.

There is also provided the use of a compound of general formula (I) in the preparation of an agent for the treatment or prevention of a mycobacterial condition.

The compound may be used for the treatment or prevention of tuberculosis or leprosy but preferably the compounds of formula (I) are used in the treatment or prevention of tuberculosis.

Therefore the invention further comprises a method for the treatment or prevention of tuberculosis, the method comprising administering to a patient in need of such treatment an effective amount of a compound of general formula (I).

Pyrimidine and quinazoline compounds similar to those of general formula (I) are known in the art and have been used for many different purposes, both as pharmaceuticals and for other purposes.

For example, WO 2006/105056 relates to compounds in which a pyrimidine ring substituted with amino groups is fused to another ring. These compounds are said to be useful as insecticides. U.S. Pat. No. 5,534,518 also relates to insecticidal compounds.

WO 2006/097441 relates to the use of quinazoline compounds as potassium channel modulating agents.

WO 2006/071095 teaches a method for the preparation of quinazoline compounds which are useful in the treatment of diabetes and obesity. WO 03/028641 also relates to quinazoline derivatives which are useful in the treatment of obesity.

WO 2006/050843 relates to quinazoline compounds which are PTP-1B inhibitors.

There are a number of documents which relate to the use of quinazoline derivatives as phosphodiesterase inhibitors, for example PDE4, PDE5, PDE7 and PDE10 inhibitors. These include WO 2006/026395, WO 02/102315, WO 02/088080, U.S. Pat. No. 6,331,543, EP 1097711.

US 2006/025406 teaches the use of 2,4-diaminoquinalzolines as modulators of hepatocyte growth factor which are useful in the treatment of cancer. Other document which teach similar compounds as anti-cancer agents include US 2004/229890 and WO 2004/099159, which relate to quinazoline diamine derivatives with protein tyrosine phosphatase inhibiting activity and WO 03/05586, which teaches compounds which are inhibitors of polylpeptidase and inducers of apoptosis. US 2002/025968 relates to the inhibition of neoplastic cells and U.S. Pat. No. 6,262,059 and U.S. Pat. No. 6,046,206 both relate to the treatment of precancerous lesions. WO 92/14716 and WO 92/07844 both relate to the use of quinazoline compounds for enhancing antitumour activity.

WO2005/082865 relates to bicyclic pyrimidine derivatives for treating inflammatory diseases and other conditions mediated by CCR4.

There are also several prior art documents which relate to the use of quinazolines and similar compounds for treating digestive disorders or ulcers. These include WO 99/50264, U.S. Pat. No. 5,064,833 and WO 89/05297.

There are a number of documents which relate to quinazolines or pyrimidine derivatives as protein kinase inhibitors. These include US 2005/038023, which relates to pyrazole derivatives of quinazolines or other bicyclic pyrimidine derivatives. In addition, WO 02/062789, WO 02/059111, WO 02/022601, WO 02/022602, WO 02/022603, WO 02/022604, WO 02/022605, WO 02/022606, WO 02/022607, WO 02/022608, WO 02/050065 and WO02/057259 all relate to compounds protein kinase inhibitory activity.

WO 2005/011758 relates to the use of pyrimidine and quinazoline derivatives as antimicrobials, particularly bactericides and fungicides. The compounds are said to be useful as preservatives.

U.S. Pat. No. 5,439,895, U.S. Pat. No. 5,436,233 and EP 0579496 all relate to the use of quinazolines as cGMP phosphodiesterase and TXA2 synthase inhibitors.

It is clear from these prior art that numerous pyrimidine and quinazoline compounds are known and that the compounds have a large number of uses. However, none of the above prior art documents teaches or suggests that these compounds might be of use in the treatment of bacterial infections, especially tuberculosis.

There are various references which teach the use of quinazoline compounds for the treatment of bacterial infections.

Thayer et al, Antibiotics and Chemotherapy, vol II No. 9, 463-466, (1952) relates to three quinazoline compounds which were known for the treatment of malaria and which the author suggested could be used in the treatment of mycobacterial infections. These compounds are 2(1-ethyl-3-guanidino)-4-methyl-6-chloroquinazoline hydrochloride hydrate, 2(dimethylamino)-4-amino-6,7-dimethoxyquinazoline dihydrochloride and 2(1-isopropyl-3-guanidino)-4-methyl-6-chloroquinazoline nitrate. None of these compounds is particularly similar to the compounds of the present invention.

De La Fuente et al, British Journal of Pharmacology, (2006), 149, 551-559 relates to compounds which are said to have activity against E. coli and P. aeruginosa. Some of the compounds are similar to the compounds of the present invention but there is no suggestion in this document that they would be of use in the treatment of mycobacterial infections such as TB.

Kunes et al, II Farmaco 55 (2000), 725-729 relates to quinazoline derivatives which are said to have anti tubercular activity. These compounds are significantly different from the compounds of the present invention as they have no substituent in a position equivalent to NR³R⁴ and have an SR substituent in place of NR¹R².

GB664262 relates to 2,4-diaminoquinazoline compounds in which one of the amino groups is bound directly to a carbon atom at the 2-position of a thiazole or imidazole ring and the other contains an organic substituent having a tertiary amino group. These compounds are said to have anti-TB activity. The exemplified compounds all have a diethylamino alkylamino group at either the 2- or the 4-position of the quinazoline ring and the authors suggest that this type of substitution pattern is necessary for anti-TB activity.

Le et al, Bull. Korean Chem. Soc. (2007), 28(6), 947-952 relates to the analysis of structural models of compounds likely to be inhibitors of tubercular acetohydroxy acid synthase. However, the authors of this paper have not actually synthesised or tested any of compounds but merely suggested that they may have the correct stereochemistry to fit the authors' model.

WO 03/099820 relates to compounds which are said to be of use for treating p38 kinase-associated conditions and the list of conditions includes tuberculosis. However, the document contains no examples and no experimental evidence to demonstrate that the compounds would have the suggested use. In addition, it is not the case that inhibitors of p38 MAPK would be expected to have a direct bactericidal or bacteriostatic effect as there is no p38 MAPK or equivalent enzyme in M. tuberculosis.

M. tuberculosis survives and is able to persist in the host by parasitizing macrophages and arresting phagosome maturation and a key part of this process is activation of the human p38 MAPK by the m. tuberculosis bacteria. (For example, see R. Fratti et al. Journal of Biological Chemistry 2003, 278(47), pp 46961-46967.) As such, inhibitors of p38 MAPK could be expected to moderate the human immune response to infection but would not be expected to have a direct bactericidal or bacteriostatic effect. However, the inventors have found that the compounds of the present invention have a direct growth inhibition effect on TB bacteria in vitro (i.e. in the absence of any human immune cells/system).

A number of references teach the use of dihydrofolate reductase inhibitors for the treatment of TB. Dihydrofolate reductase inhibitor compounds are well known and all have certain common structural features. In general, such compounds are pyrimidine compounds with NH₂ substituents at the 2- and 4-positions and a bulky substituent at the 5-position or quinazoline compounds with NH₂ substituents at the 2- and 4-positions and a bulky substituent in the 6-position.

EP 0255100 describes the use of trimetrexate (5-methyl-6-{[(3.4.5-trimethoxyphenyl)amino]methyl}-2,4-quinazolinediamine for the treatment of infections of the Mycobacterium avium intracellulare complex. EP 0542497 relates to pyrroloquinazoline derivatives which are said to be dihydrofolate reductase inhibitors and to be useful in the treatment of bacterial (including mycobacterial) infections in mammals. WO 2004/082613 relates to pyrimidine and quinazoline compounds which are said to have dihydrofolate reductase inhibiting activity, including inhibition of dihydrofolate reductase of Mycobacterium avium, and to be useful for treating mycobacterial infections. All of the compounds described in these documents have the structural features typical of dihydrofolate reductase inhibitors.

In contrast, the compounds of the present invention do not have these structural features. In particular, they all have a group NR³R⁴ at the 2-position and a group NR¹R² at the 4-position. R² and R⁴ cannot be hydrogen and therefore the compounds of general formula (I) lack an important feature common to dihydrofolate reductase inhibitors.

Surprisingly, however, the compounds of general formula (I) were compared with similar compounds in which either or both of NR¹R² and NR³R⁴ was replaced with NH₂, it was found that the compounds of general formula (I) were significantly more active in both a minimum inhibition concentration assay and a low oxygen recovery assay (see Examples 2 and 3 below).

In the present specification “C₁-C₆ alkyl” refers to a straight or branched saturated hydrocarbon chain having one to six carbon atoms. Examples include methyl, ethyl, n-propyl, isopropyl, t-butyl, n-hexyl.

“C₁-C₄ alkyl” has a similar meaning except that it contains from one to four carbon atoms.

“C₂-C₆ alkenyl” refers to a straight or branched hydrocarbon chain having from two to six carbon atoms and containing at least one carbon-carbon double bond. Examples include ethenyl, 2-propenyl, and 3-hexenyl.

The term “C₁-C₆ haloalkyl” refers to a C₁₋₆ alkyl group as defined above substituted by one or more halogen atoms.

The terms “carbocyclic ring system” and “carbocyclyl” refers to a 3 to 14 membered carbocyclic ring, (except when alternative numbers of ring atoms are specified), which may be fully or partially saturated and which includes fused bicyclic or tricyclic systems. Examples of such groups include cyclopropyl, cyclobutyl, cyclopentyl, cyclohexyl, cyclohexenyl and also bridged systems such as norbornyl and adamantyl.

The terms “heterocyclic ring system” and “heterocyclyl” refers to a saturated or partially saturated 3 to 14 membered ring system (except when alternative numbers of ring atoms are specified) similar to cycloalkyl but in which at least one of the carbon atoms has been replaced by N, O, S, SO or SO₂. Examples include piperidine, piperazine, morpholine, tetrahydrofuran and pyrrolidine,

The terms “aryl” and “aromatic moiety” in the context of the present specification refer to an aromatic ring system having from 6 to 14 ring carbon atoms (except when other numbers of ring atoms are specified) and containing up to three rings. Examples of aromatic moieties are benzene and naphthalene. The term also includes bicyclic or tricyclic systems in which one or more of the rings has aromatic character. Indane is an example of this type of system.

The terms “heteroaryl” and “heteroaromatic moiety” refer to an aromatic ring system, which may be partially saturated and which has from 5 to 14 ring atoms (except when other numbers of atoms are specified) and containing up to three rings and at least one heteroatom selected from N, O and S. The term also includes systems in which a ring having aromatic character is fused to a saturated or partially saturated ring. Examples include pyridine, pyrimidine, furan, thiophene, indole, isoindole, indoline, benzofuran, benzimidazole, benzimidazoline quinoline, isoquinoline, tetrahydroisoquinoline, quinazoline, thiazole, benzthiazole, benzoxazole, indazole and imidazole ring systems.

In the present specification, “halo” refers to fluoro, chloro, bromo or iodo.

Appropriate pharmaceutically and veterinarily acceptable salts of the compounds of general formula (I) include basic addition salts such as sodium, potassium, calcium, aluminium, zinc, magnesium and other metal salts as well as choline, diethanolamine, ethanolamine, ethyl diamine and other well known basic addition salts.

Where appropriate, pharmaceutically or veterinarily acceptable salts may also include salts of organic acids, especially carboxylic acids, including but not limited to acetate, trifluoroacetate, lactate, gluconate, citrate, tartrate, maleate, malate, pantothenate, adipate, alginate, aspartate, benzoate, butyrate, digluconate, cyclopentanate, glucoheptanate, glycerophosphate, oxalate, heptanoate, hexanoate, fumarate, nicotinate, pamoate, pectinate, 3-phenylpropionate, picrate, pivalate, proprionate, tartrate, lactobionate, pivolate, camphorate, undecanoate and succinate, organic sulfonic acids such as methanesulfonate, ethanesulfonate, 2-hydroxyethane sulfonate, camphorsulfonate, 2-naphthalenesulfonate, benzenesulfonate, p-chlorobenzenesulfonate and p-toluenesulfonate; and inorganic acids such as hydrochloride, hydrobromide, hydroiodide, sulfate, bisulfate, hemisulfate, thiocyanate, persulfate, phosphoric and sulfonic acids.

Salts which are not pharmaceutically or veterinarily acceptable may still be valuable as intermediates.

Prodrugs are any covalently bonded compounds which release the active parent drug according to general formula (I) in vivo.

If a chiral centre or another form of isomeric centre is present in a compound of the present invention, all forms of such isomer or isomers, including enantiomers and diastereoisomers, are intended to be covered herein. Compounds of the invention containing a chiral centre may be used as a racemic mixture, an enantiomerically enriched mixture, or the racemic mixture may be separated using well-known techniques and an individual enantiomer may be used alone.

The compounds of the present invention have been shown to inhibit the growth of Mycobacterium tuberculosis in a standard MABA assay (Collins et al, Antimicrobial Agents and Chemotherapy., (1997), 1004-1009).

Perhaps the most surprising and potentially useful effect of the compounds of the present invention is that they are active against the dormant, or non replicating persistent, phase of M. tuberculosis infection. As reported by Cho et al, (Antimicrobial Agents and Chemotherapy, (2007) 1380-1385), it is widely accepted that a state of non replicating persistence is responsible for antimicrobial tolerance in many bacterial infections, including TB. Cho et al describe an assay for the high thoughput screening of compounds against non replicating M. tuberculosis and the compounds of the present invention have shown activity in this assay, indicating that they are likely to be of use in the treatment of the latent or persistent phase of TB.

In suitable compounds of general formula (I), independently or in any combination:

A is phenyl.

In quinoline compounds of general formula (I), A is phenyl, X¹ is CH and X² is N, while in isoquinoline compounds of general formula (I), A is phenyl X¹ is N and X² is CH. In some cases, the quinoline compounds are preferred over isoquinoline compounds because they are easier to synthesise.

In more suitable compounds of the present invention, both X¹ and X² are N and examples of such compounds are quinazolines, where A is phenyl.

In some example compounds of the present invention independently or in any combination:

R¹ is hydrogen or C₁-C₄ alkyl, optionally substituted with phenyl; and in particular R¹ is hydrogen, methyl or benzyl;
R² is a carbocyclic moiety; or
a group —C₁-C₄ alkyl-R⁵, where R⁵ is an aryl, heteroaryl, carbocyclyl or heterocyclyl group optionally substituted with halo, CN, NO₂, C₁-C₄ alkyl, C₁-C₄ haloalkyl, O(C₁-C₄ alkyl), N(C₁-C₄ alkyl)₂, CO(C₁-C₄ alkyl), CO₂(C₁-C₄ alkyl) or SO₂(C₁-C₄ alkyl).

Examples of suitable carbocyclic groups for R² include adamantyl and, when R² is C₁-C₄ alkyl-R⁵, R⁵ may be, for example phenyl, thiophene, pyridine, naphthalene, indane, cyclohexyl or furyl optionally substituted with one or more substituents chosen from chloro, fluoro, trifluoromethyl, dimethylamino, methoxy, methyl, ethyl, CO₂CH₃, nitrile and SO₂CH₃.

Alternatively, R¹ and R² together may form a heterocyclic ring system and examples of suitable rings include isoindoline, piperazine, piperidine, dihydroisoquinoline, indene and indane any of which may optionally be substituted with one or more phenyl or halophenyl groups.

In some example compounds of the present invention, independently or in any combination:

R³ is hydrogen or C₁-C₄ alkyl, especially, hydrogen, methyl or ethyl; and
R⁴ is R⁵, COR⁵ or C₁-C₄ alkyl or C₂-C₄ alkenyl optionally substituted with R⁵, or NHR⁵,
where R⁵ is aryl or heteroaryl, especially phenyl, optionally substituted with C₁-C₄ alkyl, halo or NO₂.

It is, however, preferred that when one of R² and R⁴ is CH₂R⁵ and R⁵ is furanyl or tetrahydrofuranyl, the other of R² and R⁴ is not unsubstituted phenyl or phenyl substituted with OH.

More usually, R³ and R⁴ together form a heterocyclyl group especially a 5- to 7-membered heterocyclic group such as piperidine, pyrrolidine, morpholine or a 7-membered ring containing an additional nitrogen or oxygen atom; or such a group fused to a phenyl group, for example a tetrahydroisoquinoline group. Any of these may be substituted with groups such as CO(C₄-C₇ cycloalkyl), CO-aryl, CO(C₁-C₄ alkyl), CO₂(C₄-C₇ cycloalkyl), CO₂-aryl, CO₂(C₁-C₄ alkyl), SO₂(C₄-C₇ cycloalkyl), SO₂-aryl, SO₂(C₁-C₄ alkyl) or CH₂CO(C₁-C₄ alkyl).

Particularly preferred compounds of general formula (I) are:
N-Benzyl-2-(piperidin-1-yl)quinazolin-4-amine
N-Benzyl-2-morpholinoquinazolin-4-amine
N-Benzyl-2-(4-methylpiperazin-1-yl)quinazolin-4-amine
N¹-(2-Morpholinoquinazolin-4-yl)-N²-(4-nitrophenyl)ethane-1,2-diamine
N-(1-Phenylethyl)-2-(piperazin-1-yl)quinazolin-4-amine
2-Morpholino-N-(1-phenylethyl)quinazolin-4-amine
2-(4-(Methylsulfonyl)piperazin-1-yl)-N-(1-phenylethyl)quinazolin-4-amine
N¹-(2-(4-Methylpiperazin-1-yl)quinazolin-4-yl)-N²-(4-nitrophenyl)ethane-1,2-diamine
N-(1-Phenylethyl)-2-(piperidin-1-yl)quinazolin-4-amine
N-(Furan-2-ylmethyl)-2-(piperidin-1-yl)quinazolin-4-amine
N-(1-Phenylethyl)-2-(pyrrolidin-1-yl)quinazolin-4-amine
N-(1-Phenylethyl)-2-(4-(phenylsulfonyl)piperazin-1-yl)quinazolin-4-amine
N-(2-Adamantyl)-2-morpholinoquinazolin-4-amine
N-Benzyl-6,7-dimethoxy-2-morpholinoquinazolin-4-amine
N²,N⁴-Dibenzylquinazoline-2,4-diamine
N-(1-(2-Adamantyl)ethyl)-2-morpholinoquinazolin-4-amine
N-Benzyl-1-methyl-6-(pyrrolidin-1-yl)-1H-pyrazolo[3,4-d]pyrimidin-4-amine
Cyclopentyl(4-(4-(1-phenylethylamino)quinazolin-2-yl)piperazin-1-yl)methanone
N-Benzyl-2-(pyrrolidin-1-yl)quinazolin-4-amine
N⁴-Benzyl-N²-phenylquinazoline-2,4-diamine
N-Benzyl-4-(piperidin-1-yl)quinazolin-2-amine
N⁴-Benzyl-N², N²-diethylquinazoline-2,4-diamine
N-Benzyl-2-(4-(ethylsulfonyl)piperazin-1-yl)quinazolin-4-amine
Ethyl 2-(4-(4-(benzylamino)quinazolin-2-yl)piperazin-1-yl)acetate
N⁴-Benzyl-N²-(4-methylbenzyl)quinazoline-2,4-diamine
N²,N⁴-Dibenzyl-N²-methylquinazoline-2,4-diamine
N-Benzyl-2-(3,4-dihydroisoquinolin-2(1H)-yl)quinazolin-4-amine
tert-Butyl 4-(4-(benzylamino)quinazolin-2-yl)piperazine-1-carboxylate
(S)—N-(1-Phenylethyl)-2-(piperidin-1-yl)quinazolin-4-amine
N-Phenethyl-2-(piperidin-1-yl)quinazolin-4-amine
N-(4-Chlorobenzyl)-2-(piperidin-1-yl)quinazolin-4-amine
N-Benzyl-N-methyl-2-(piperidin-1-yl)quinazolin-4-amine
4-(3,4-Dihydroisoquinolin-2(1H)-yl)-2-(piperidin-1-yl)quinazoline
N-(Naphthalen-1-ylmethyl)-2-(piperidin-1-yl)quinazolin-4-amine
2-(Piperidin-1-yl)-N-(4-(trifluoromethyl)benzyl)quinazolin-4-amine
(R)—N-(1-Phenylethyl)-2-(piperidin-1-yl)quinazolin-4-amine
N-(2-Chlorobenzyl)-2-(piperidin-1-yl)quinazolin-4-amine
N-(3-Chlorobenzyl)-2-(piperidin-1-yl)quinazolin-4-amine
2-(Piperidin-1-yl)-N-(2-(trifluoromethyl)benzyl)quinazolin-4-amine
N-(4-Methylbenzyl)-2-(piperidin-1-yl)quinazolin-4-amine
N-(4-Methoxybenzyl)-2-(piperidin-1-yl)quinazolin-4-amine
N-(Cyclohexylmethyl)-2-(piperidin-1-yl)quinazolin-4-amine
2-(Piperidin-1-yl)-N-(pyridin-2-ylmethyl)quinazolin-4-amine
2-(Piperidin-1-yl)-N-(pyridin-3-ylmethyl)quinazolin-4-amine
2-(Piperidin-1-yl)-N-(thiophen-2-ylmethyl)quinazolin-4-amine
2-(Piperidin-1-yl)-N-(3-(trifluoromethyl)benzyl)quinazolin-4-amine
N-Benzhydryl-2-(piperidin-1-yl)quinazolin-4-amine
N-Benzyl-2-(piperazin-1-yl)quinazolin-4-amine
N-Benzyl-2-(1,4-oxazepan-4-yl)quinazolin-4-amine
N-(2-Phenylpropan-2-yl)-2-(piperidin-1-yl)quinazolin-4-amine
N-Benzyl-2-(piperidin-1-yl)quinolin-4-amine
tert-Butyl 4-(4-(benzylamino)quinazolin-2-yl)-1,4-diazepane-1-carboxylate
1-(4-(4-(Benzylamino)quinazolin-2-yl)piperazin-1-yl)-2-methylpropan-1-one
1-(4-(4-(Benzylamino)quinazolin-2-yl)piperazin-1-yl)-2,2-dimethylpropan-1-one
(4-(4-(Benzylamino)quinazolin-2-yl)piperazin-1-yl)(phenyl)methanone
N-(1-Adamantyl)-2-(piperidin-1-yl)quinazolin-4-amine
N-(2-Adamantyl)-2-(piperidin-1-yl)quinazolin-4-amine
N-(1-Adamantyl)-2-morpholinoquinazolin-4-amine
N-(4-(Dimethylamino)benzyl)-2-(piperidin-1-yl)quinazolin-4-amine
N-(4-Fluorobenzyl)-2-(piperidin-1-yl)quinazolin-4-amine
N-(3-Fluorobenzyl)-2-(piperidin-1-yl)quinazolin-4-amine
N-(2-Fluorobenzyl)-2-(piperidin-1-yl)quinazolin-4-amine
N,N-Dibenzyl-2-(piperidin-1-yl)quinazolin-4-amine
N⁴-Benzyl-N²-propylquinazoline-2,4-diamine
N-(3,4-Dichlorobenzyl)-2-(piperidin-1-yl)quinazolin-4-amine
N-(2,4-Dichlorobenzyl)-2-(piperidin-1-yl)quinazolin-4-amine
N-Benzyl-6,7-dimethoxy-2-(piperidin-1-yl)quinazolin-4-amine
N-Benzyl-6-chloro-2-(piperidin-1-yl)quinazolin-4-amine
N-Phenyl-2-(piperidin-1-yl)quinazolin-4-amine
N-(4-(Benzylamino)quinazolin-2-yl)benzamide
N-(4-Chlorobenzyl)-2-morpholinoquinazolin-4-amine
N-(4-Chlorobenzyl)-2-(pyrrolidin-1-yl)quinazolin-4-amine
N⁴-(4-Chlorobenzyl)-N²-(4-methylbenzyl)quinazoline-2,4-diamine
2-Morpholino-N-(4-(trifluoromethyl)benzyl)quinazolin-4-amine
2-(Pyrrolidin-1-yl)-N-(4-(trifluoromethyl)benzyl)quinazolin-4-amine
N²-(4-Methylbenzyl)-N⁴-(4-(trifluoromethyl)benzyl)quinazoline-2,4-diamine
N-(3-Methylbenzyl)-2-(piperidin-1-yl)quinazolin-4-amine
N-(2-Methoxybenzyl)-2-(piperidin-1-yl)quinazolin-4-amine
N-(3-Methoxybenzyl)-2-(piperidin-1-yl)quinazolin-4-amine
N-(4-Chlorophenethyl)-2-(piperidin-1-yl)quinazolin-4-amine
N-(4-Methoxyphenethyl)-2-(piperidin-1-yl)quinazolin-4-amine
4-(Isoindolin-2-yl)-2-(piperidin-1-yl)quinazoline
N-(2,3-Dihydro-1H-inden-2-yl)-2-(piperidin-1-yl)quinazolin-4-amine
N-(4-Methylphenethyl)-2-(piperidin-1-yl)quinazolin-4-amine
4-(4-Phenylpiperazin-1-yl)-2-(piperidin-1-yl)quinazoline
4-(4-(4-Chlorophenyl)piperazin-1-yl)-2-(piperidin-1-yl)quinazoline
4-(4-Phenylpiperidin-1-yl)-2-(piperidin-1-yl)quinazoline
N-(2-Phenylpropan-2-yl)-2-(pyrrolidin-1-yl)quinazolin-4-amine
4-((2-(Piperidin-1-yl)quinazolin-4-ylamino)methyl)benzonitrile
Methyl 4-((2-(piperidin-1-yl)quinazolin-4-ylamino)methyl)benzoate
N-(4-(Methylsulfonyl)benzyl)-2-(piperidin-1-yl)quinazolin-4-amine
N-(3-Phenylpropyl)-2-(piperidin-1-yl)quinazolin-4-amine
(R)—N-(1-(4-Chlorophenyl)ethyl)-2-(piperidin-1-yl)quinazolin-4-amine
(R)—N-(1-(4-Methoxyphenyl)ethyl)-2-(piperidin-1-yl)quinazolin-4-amine
(R)—N-(1-(4-Fluorophenyl)ethyl)-2-(piperidin-1-yl)quinazolin-4-amine
(R)—N-(1-(Naphthalen-1-yl)ethyl)-2-(piperidin-1-yl)quinazolin-4-amine
(R)—N-(1-(Naphthalen-2-yl)ethyl)-2-(piperidin-1-yl)quinazolin-4-amine
(R)-2-(Piperidin-1-yl)-N-(1-p-tolylethyl)quinazolin-4-amine
2-(Piperidin-1-yl)-N-(2-p-tolylpropan-2-yl)quinazolin-4-amine
2-(Pyrrolidin-1-yl)-N-(2-p-tolylpropan-2-yl)quinazolin-4-amine
N-(2-Methylbenzyl)-2-(piperidin-1-yl)quinazolin-4-amine
2-(Piperidin-1-yl)-N-(4-(trifluoromethyl)phenethyl)quinazolin-4-amine
N-(4-Chlorobenzyl)-2-(3,4-dihydroisoquinolin-2(1H)-yl)quinazolin-4-amine
N-Benzyl-5-chloro-2-(piperidin-1-yl)quinazolin-4-amine
N-(1-(4-Fluorophenyl)-2-methylpropan-2-yl)-2-(piperidin-1-yl)quinazolin-4-amine
4-(Piperidin-1-yl)-N-(4-(trifluoromethyl)benzyl)quinazolin-2-amine
N-(4-Chlorobenzyl)-4-(piperidin-1-yl)quinazolin-2-amine
N-(4-Fluorobenzyl)-4-(piperidin-1-yl)quinazolin-2-amine
N-(2,4-Dichlorobenzyl)-4-(piperidin-1-yl)quinazolin-2-amine
2-(3,4-Dihydroisoquinolin-2(1H)-yl)-4-(piperidin-1-yl)quinazoline
2-(3,4-Dihydroisoquinolin-2(1H)-yl)-N-(4-(trifluoromethyl)benzyl)quinazolin-4-amine
N-(2,4-Difluorobenzyl)-2-(piperidin-1-yl)quinazolin-4-amine
N-(3,4-Difluorobenzyl)-2-(piperidin-1-yl)quinazolin-4-amine
(R)-2-(Piperidin-1-yl)-N-(1-(4-(trifluoromethyl)phenyl)ethyl)quinazolin-4-amine
N⁴-(4-Fluorobenzyl)-N²-(4-fluorophenyl)quinazoline-2,4-diamine
N²,N⁴-bis(4-Fluorobenzyl)quinazoline-2,4-diamine
N-(2-(4-Fluorophenyl)propan-2-yl)-2-(piperidin-1-yl)quinazolin-4-amine
N-(2-(4-Fluorophenyl)propan-2-yl)-2-(pyrrolidin-1-yl)quinazolin-4-amine
N-(1-Phenylcyclopropyl)-2-(piperidin-1-yl)quinazolin-4-amine
N-(1-Phenylcyclopropyl)-2-(pyrrolidin-1-yl)quinazolin-4-amine
N-(3-Phenylpentan-3-yl)-2-(pyrrolidin-1-yl)quinazolin-4-amine
N-(1-Phenylcyclohexyl)-2-(pyrrolidin-1-yl)quinazolin-4-amine
N4-Benzyl-N²-(4-fluorobenzyl)quinazoline-2,4-diamine
N4-Benzyl-N²-(4-chlorobenzyl)quinazoline-2,4-diamine
N4-Benzyl-N²-(4-(trifluoromethyl)benzyl)quinazoline-2,4-diamine
N4-Benzyl-N²-(4-methoxybenzyl)quinazoline-2,4-diamine
N-Benzyl-2-(isoindolin-2-yl)quinazolin-4-amine
N4-Benzyl-N²-(2,3-dihydro-1H-inden-2-yl)quinazoline-2,4-diamine
N-Benzyl-2-(4-phenylpiperidin-1-yl)quinazolin-4-amine
N-Benzyl-2-(4-phenylpiperazin-1-yl)quinazolin-4-amine
N-Benzyl-2-(4-(4-chlorophenyl)piperazin-1-yl)quinazolin-4-amine
N-Benzyl-2-(4-(4-(trifluoromethyl)phenyl)piperazin-1-yl)quinazolin-4-amine
N-Benzyl-2-(4-(4-methoxyphenyl)piperazin-1-yl)quinazolin-4-amine
2-(4,4-Difluoropiperidin-1-yl)-N-(4-fluorobenzyl)quinazolin-4-amine
N-(3-Phenylpentan-3-yl)-2-(piperidin-1-yl)quinazolin-4-amine
1-(4-(Benzylamino)quinazolin-2-yl)piperidin-4-one
N-Benzyl-2-thiomorpholinoquinazolin-4-amine
N-(1-(4-Chlorophenyl)cyclopropyl)-2-(piperidin-1-yl)quinazolin-4-amine
N-(1-(4-Chlorophenyl)cyclopropyl)-2-(pyrrolidin-1-yl)quinazolin-4-amine
(S)-3-Phenyl-2-(2-(piperidin-1-yl)quinazolin-4-ylamino)propan-1-ol
(S)—N-(1-Methoxy-3-phenylpropan-2-yl)-2-(piperidin-1-yl)quinazolin-4-amine
(R)-2-Phenyl-2-(2-(piperidin-1-yl)quinazolin-4-ylamino)ethanol
N-(4-Chlorobenzyl)-4-(4,4-difluoropiperidin-1-yl)quinazolin-2-amine
4-(4,4-Difluoropiperidin-1-yl)-N-(4-(trifluoromethyl)benzyl)quinazolin-2-amine
N-(2,4-Difluorobenzyl)-4-(4,4-difluoropiperidin-1-yl)quinazolin-2-amine
4-(4,4-Difluoropiperidin-1-yl)-N-(4-fluorobenzyl)quinazolin-2-amine
N-(4-Fluorobenzyl)-6,7-dimethoxy-2-(piperidin-1-yl)quinazolin-4-amine
2-(4,4-Difluoropiperidin-1-yl)-N-(4-fluorobenzyl)-6,7-dimethoxyquinazolin-4-amine
N-(1-(4-Fluorophenyl)cyclopropyl)-2-(piperidin-1-yl)quinazolin-4-amine
2-(4,4-Difluoropiperidin-1-yl)-N-(1-(4-fluorophenyl)cyclopropyl)quinazolin-4-amine
N²-(4-Fluorobenzyl)-N⁴-(1-(4-fluorophenyl)cyclopropyl)quinazoline-2,4-diamine
7-Fluoro-N-(4-fluorobenzyl)-2-(piperidin-1-yl)quinazolin-4-amine
2-(4,4-Difluoropiperidin-1-yl)-7-fluoro-N-(4-fluorobenzyl)quinazolin-4-amine
7-Fluoro-N²,N⁴-bis(4-fluorobenzyl)quinazoline-2,4-diamine
N²-(4-Fluorobenzyl)-N⁴-((5-methylfuran-2-yl)methyl)quinazoline-2,4-diamine
N²-(4-Fluorobenzyl)-N⁴-((5-(trifluoromethyl)furan-2-yl)methyl)quinazoline-2,4-diamine
2-(Piperidin-1-yl)-N-((5-(trifluoromethyl)furan-2-yl)methyl)quinazolin-4-amine
2-(4,4-Difluoropiperidin-1-yl)-N-((5-(trifluoromethyl)furan-2-yl)methyl)quinazolin-4-amine
2-(Piperidin-1-yl)-N-(4-(trifluoromethoxy)benzyl)quinazolin-4-amine
2-(4,4-Difluoropiperidin-1-yl)-N-(4-(trifluoromethoxy)benzyl)quinazolin-4-amine
4-((2-(Piperidin-1-yl)quinazolin-4-ylamino)methyl)phenol
4-((2-(4-Fluorobenzylamino)quinazolin-4-ylamino)methyl)phenol
N², N⁴-bis(4-Fluorobenzyl)thieno[3,2-d]pyrimidine-2,4-diamine
N-((5-Methylfuran-2-yl)methyl)-2-(piperidin-1-yl)quinazolin-4-amine
N²,N⁴-bis(4-Fluorobenzyl)-6,7-dimethoxyquinazoline-2,4-diamine
N-(4-Fluorobenzyl)-2-morpholinoquinazolin-4-amine
N⁴-(4-Fluorobenzyl)-N²-((5-methylfuran-2-yl)methyl)quinazoline-2,4-diamine
N²-(Benzo[b]thiophen-2-ylmethyl)-N⁴-(4-fluorobenzyl)quinazoline-2,4-diamine
N-(4-Fluorobenzyl)-2-(pyrrolidin-1-yl)quinazolin-4-amine
N-(4-Fluorobenzyl)-2-(pyrrolidin-1-yl)pyrido[2,3-d]pyrimidin-4-amine
N-(4-Fluorobenzyl)-2-(piperidin-1-yl)pyrido[2,3-d]pyrimidin-4-amine
(S)-Methyl 2-phenyl-2-(2-(piperidin-1-yl)quinazolin-4-ylamino)acetate
(S)-Methyl 3-phenyl-2-(2-(piperidin-1-yl)quinazolin-4-ylamino)propanoate
(S)-(1-(4-(Benzylamino)quinazolin-2-yl)pyrrolidin-2-yl)methanol
(S)—N-Benzyl-2-(2-(methoxymethyl)pyrrolidin-1-yl)quinazolin-4-amine
N-(4-Fluorobenzyl)-2-(isoindolin-2-yl)quinazolin-4-amine
(S)-Methyl 1-(4-(benzylamino)quinazolin-2-yl)pyrrolidine-2-carboxylate
N-Benzyl-2-(6,7-dimethoxy-3,4-dihydroisoquinolin-2(1H)-yl)quinazolin-4-amine
5-Chloro-N-(4-fluorobenzyl)-2-(pyrrolidin-1-yl)quinazolin-4-amine
5-Chloro-N-(4-fluorobenzyl)-2-(piperidin-1-yl)quinazolin-4-amine
tert-Butyl-4-(4-fluorobenzylamino)-2-(piperidin-1-yl)-5,6-dihydropyrido-[3,4-d]pyrimidine-7(8H)-carboxylate
N-(4-Fluorophenyl)-2-(pyrrolidin-1-yl)pteridin-4-amine
4-(3,4-Dihydroisoquinolin-2(1H)-yl)-2-(pyrrolidin-1-yl)quinazoline
2-(Pyrrolidin-1-yl)-N-(thiophen-2-ylmethyl)quinazolin-4-amine
4-(6,7-Dimethoxy-3,4-dihydroisoquinolin-2(1H)-yl)-2-(pyrrolidin-1-yl)quinazoline
N-((5-Chlorothiophen-2-yl)methyl)-2-(piperidin-1-yl)quinazolin-4-amine
N-(Furan-3-ylmethyl)-2-(piperidin-1-yl)quinazolin-4-amine
2-(Piperidin-1-yl)-N-(thiophen-3-ylmethyl)quinazolin-4-amine
4-(6,7-Dimethoxy-3,4-dihydroisoquinolin-2(1H)-yl)-2-(piperidin-1-yl)quinazoline
2-(7-Bromo-3,4-dihydroisoquinolin-2(1H)-yl)-N-(4-fluorobenzyl)quinazolin-4-amine
N-((6-Chloropyridin-3-yl)methyl)-2-(piperidin-1-yl)quinazolin-4-amine
2-(Piperidin-1-yl)-N-(quinolin-4-ylmethyl)quinazolin-4-amine
2-(Piperidin-1-yl)-N-((6-(trifluoromethyl)pyridin-3-yl)methyl)quinazolin-4-amine
2-(Azepan-1-yl)-N-benzylquinazolin-4-amine
(S)-(1-(4-(4-Fluorobenzylamino)quinazolin-2-yl)pyrrolidin-2-yl)methanol
(S)—N-(4-Fluorobenzyl)-2-(2-(methoxymethyl)pyrrolidin-1-yl)quinazolin-4-amine
(S)-Methyl 1-(4-(4-fluorobenzylamino)quinazolin-2-yl)pyrrolidine-2-carboxylate
2-(4-Benzylpiperazin-1-yl)-N-(4-fluorobenzyl)quinazolin-4-amine
Cyclopropyl(4-(4-(4-fluorobenzylamino)quinazolin-2-yl)piperazin-1-yl)methanone
Cyclohexyl(4-(4-(4-fluorobenzylamino)quinazolin-2-yl)piperazin-1-yl)methanone
(4-(4-(4-Fluorobenzylamino)quinazolin-2-yl)piperazin-1-yl)(phenyl)methanone
(4-(4-(4-Fluorobenzylamino)quinazolin-2-yl)piperazin-1-yl)(pyrrolidin-1-yl)methanone
Ethyl 2-(4-(4-fluorobenzylamino)quinazolin-2-ylamino)acetate
N⁴-(4-Fluorobenzyl)-N²-(2-methoxyethyl)quinazoline-2,4-diamine
N²-(Cyclohexylmethyl)-N⁴-(4-fluorobenzyl)quinazoline-2,4-diamine
N⁴-(4-Fluorobenzyl)-N²-(2-(piperidin-1-yl)ethyl)quinazoline-2,4-diamine
   and pharmaceutically acceptable salts hydrates solvates, complexes or prodrugs thereof.

Certain of the compounds of general formula (I) are novel. Thus, according to the invention, we also provide those compounds of general formula (I) which are novel, together with processes for their preparation, compositions containing them, as well as their use as pharmaceuticals.

In a further aspect of the invention, there is provided the use of a compound of general formula (I) as defined above in the preparation of an anti-mycobacterial agent, particularly an agent for the treatment or prevention of tuberculosis.

Compounds of general formula (I) as defined above may be prepared from compounds of general formula (II):

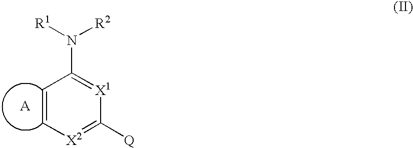

wherein A, X¹, X², R¹ and R² are as defined above for general formula (I) and Q is a leaving group, especially a halogen such as Cl;
by reaction with a compound of general formula (III):

HNR³R⁴ (III)

wherein R³ and R⁴ are as defined for general formula (I).

The reaction may be carried out in a polar organic solvent such as ethanol or actetonitrile and in some cases with microwave irradiation. There are numerous examples in the literature of this type of reaction, for example WO 2006/071095, Synthesis, 2006, 3515, J. C. S. Perkin 11992, 919, J. Med. Chem., (2003), 46, 4910, Bioorg. Med. Chem. Lett., (2006) 14, 7154 and Tet. Lett., (2000) 41, 1757.

This method may also be used when R³ and R⁴ together with the nitrogen atom to which they are attached form a 4- to 7-membered heterocyclic ring. In this case, a solvent such as acetonitrile may be preferred. In one method, the compound of general formula (II) may be treated with a weak base such as potassium carbonate before being reacted with an acid salt, for example the hydrochloride salt, of the compound of general formula (III). The reaction may be carried out at elevated temperature, for example above 150° C. and typically about 160° C. Alternatively, the compound of general formula (II) and the compound of general formula (III) may be reacted together at elevated temperature, for example greater than 150° C. and typically 175-185° C. and with microwave irradiation.

Compounds of general formula (II) are known and are commercially available or may be prepared by methods known to those of skill in the art.

Compounds of general formula (II) may be prepared from compounds of general formula (IV):

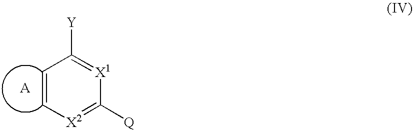

wherein X¹, X² and A are as defined for general formulae (I), Q is as defined for general formula (II) and Y is a leaving group, especially a halogen such as Cl;
by reaction with a compound of general formula (V):

HNR¹R² (V)

wherein R¹ and R² are as defined for general formula (I).

The reaction may be carried out in the presence of a base such as triethylamine and in a polar organic solvent such as tetrahydrofuran.

Compounds of general formula (IV) are commercially available or may be prepared by methods known to those of skill in the art.

For example, a compound of general formula (IV) in which both Y and Q are Cl may be prepared from a compound of general formula (VI) or its salt of general formula (VIa):

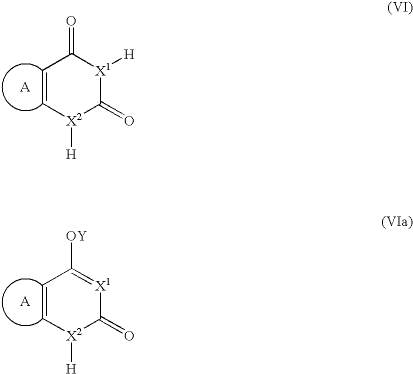

wherein X¹, X² and A are as defined for general formula (I) and Y is the ion of an alkali or alkaline earth metal such as potassium, sodium or calcium;
by reaction with POCl₃ in a polar solvent such as dimethylformamide and at elevated temperature.

Compounds of general formula (VI) and (VIa) are also known and are commercially available or can be prepared by known methods. For example, a quinazoline compound of general formula (VI) or its salt of general formula (VIa) in which both X¹ and X² are N may be prepared from a compound of general formula (VIII):

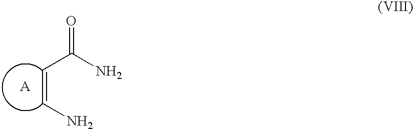

by reaction with diphenyl carbonate and potassium carbonate in an organic solvent and treatment with microwave radiation.

Alternatively, a quinazoline compound of general formula (VI) may be prepared from reaction with a compound of general formula (IX)

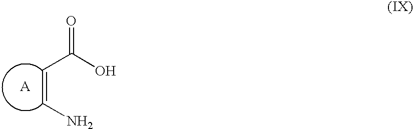

by heating with urea.

Quinoline compounds of general formula (VI) or (VIa), i.e. compounds in which X¹ is CH and X² is N, may be prepared from compounds of general formula (XIII):

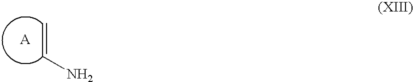

where A is as defined for general formula (I);
by reaction with propanedioic acid diethyl ester. Suitable reaction conditions are described by Shobana et al, Tetrahedron, 45(3), 757 (1989).

Isoquinoline compounds of general formula (VI) or (VIa), i.e. compounds in which X¹ is N and X² is CH, may be prepared from compounds of general formula (XIV)

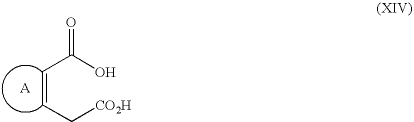

wherein A is as defined in general formula (I);
by heating with aqueous ammonium hydroxide as described in J. Med. Chem., 50(15), 3651 (2007).

Compounds of general formula (XIV) may be prepared from compounds of general formula (XV):

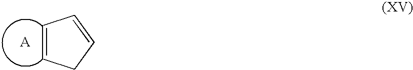

where A is as defined in general formula (I);
by reaction with OsO₄ and Jones' reagent in acetone as described in J. Org. Chem., 58(17), 4745 (1993).

Some compounds of general formula (XV), particularly indanes, are commercially available, whilst others can be prepared by well known methods.

In an alternative method, a compound of general formula (I) may be prepared from a compound of general formula (VII):

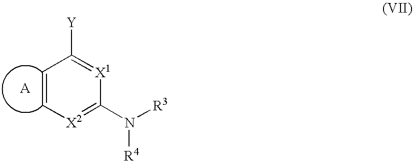

wherein A, X¹, X², R³ and R⁴ are as defined for general formula (I) and Y is as defined for general formula (IV) by reaction with a compound of general formula (V) as defined above. The reaction may sometimes be conducted in the presence of a base such as triethylamine and the reaction mixture may be heated to a temperature of from 100 to 200° C. and may be irradiated with microwave irradiation.

Compounds of general formula (VII) may be prepared by reaction of a compound of general formula (IV) as defined above with a compound of general formula (X)

R⁹NR³R⁴ (X)

wherein R⁹ is methyl or ethyl and R³ and R⁴ are as defined above.

The reaction may be carried out in a solvent such as dioxane and at elevated temperature, for example 50 to 200° C., preferably with microwave radiation.

Compounds of general formula (X) are well known to those of skill in the art and are commercially available or may be prepared by known methods.

Compounds of general formula (I) may also be prepared from other compounds of general formula (I). For example, compounds of general formula (I) in which R⁴ is a heterocyclyl group such as homopiperazine or piperazine substituted with a group COOR⁵ can be converted to a compound of general formula (I) in which R⁴ is an unsubstituted heterocyclyl group by reaction with trifluoroacetic acid.

This compound of general formula (I) can, in turn, be converted to a compound in which the heterocyclyl group R⁴ is substituted with a group COR⁵ by reaction with an acid chloride of general formula (XI):

R⁵COCl (XI)

wherein R⁵ is as defined in general formula (I);
The reaction may be carried out in the presence of a base such as triethylamine and in a polar organic solvent such as dichloromethane.

Compounds of general formula (I) in which R⁴ is COR⁵ may be prepared from compounds of general formula (XII):

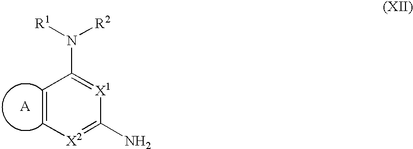

wherein R¹, R², X¹, X² and A are as defined for general formula (I);
by reaction with a compound of general formula (XI) as defined above. The reaction may be carried out in the presence of a base such as triethylamine and in a polar organic solvent such as dichloromethane at an elevated temperature of, for example about 100° C.

Compounds of general formula (XII) may be prepared from compounds of general formula (II) or (VII) in an analogous manner to the methods set out above for compounds of general formula (I).

As already outlined above, the compounds of the present invention are useful in the treatment or prevention of bacterial infection, particularly mycobacterial infection and more especially tuberculosis.

The invention therefore finds application in the treatment and prophylaxis of mycobacterial conditions associated with infection with M. tuberculosis, M. africanum, M. bovis, M. leprae, M. avium, M. intracellulare, M. scrofulaceum, M. kansasii, M xenopi, M. marinum, M. ulcerans, M. fortuitum or M. chelonae.

In preferred embodiments, the mycobacterial conditions treated or prevented according to the invention are those associated with infection by members of the Mycobacterium tuberculosis complex (MTBC), for example infection with mycobacteria selected from one or more of the species M. tuberculosis, M. bovis, M. africanum, M. canetti, M. caprae or M. pinnipedii.

In other embodiments, the invention finds application in the treatment and prophylaxis of mycobacterial conditions associated with infection by members of the Mycobacterium avium complex (MAC), for example infection with mycobacteria selected from one or more of the species M. avium, M. avium paratuberculosis, M. avium silvaticum and M. avium “hominissuis”. Such infections are a significant cause of death in AIDS patients and in other immunocompromised individuals.

Thus, the invention finds particular application in the treatment and prophylaxis of a mycobacterial condition selected from:
AIDS-related mycobacterial infection
Mycobacterial infection in immunocompromised patients (e.g. attendant on malignancy, receipt of an organ transplant, immunoablation or administration of steroids)
Pulmonary TB
Extra-pulmonary TB (including but not limited to miliary TB, central nervous system TB, pleural TB, pericardital TB, genitourinary TB, gastrointestinal TB, peritonital TB and TB of the bones and joints).
Latent (persistent or asymptomatic) mycobacterial infection
Active mycobacterial disease
MDR-TB (multidrug resistant TB)
XDR-TB (Extensive Drug Resistant TB or Extreme Drug Resistance TB): this is a recently recognized class of MDR-TB that displays resistance to three or more of the six principal classes of second-line drugs.

The compounds of the invention may therefore be used in combination with one or more additional compounds useful for the treatment of TB. Examples of such compounds include isoniazid, rifamycin and derivatives thereof, pyrazinamide, ethambutol, cycloserine, ethionamide, streptomycin, amikacin, kanamycin, capreomycin, p-aminosalicylic acid, and fluoroquinolones such as levofloxacin, moxafloxacin or gatifloxacin.

Examples of rifamycin derivatives include rifampin, rifabutin and rifapentine.

The compounds of general formula (I) may be particularly useful when used in combination with another anti-TB agent.

In a further aspect of the invention, there is provided a pharmaceutical composition comprising a compound of general formula (I) as defined above together with one or more additional compounds useful in the treatment of TB and a pharmaceutically acceptable excipient.

In yet another aspect of the invention, there is provided a product comprising a compound of general formula (I) and one or more compounds useful in the treatment of TB as a combined preparation for simultaneous, separate or sequential use in the treatment of tuberculosis.

The one or more compounds useful in the treatment of TB are preferably selected from isoniazid, rifampin, rifabutin, rifapentine, pyrazinamide, ethambutol, cycloserine, ethionamide, streptomycin, amikacin, kanamycin, capreomycin, p-aminosalicylic acid, and fluoroquinolones such as levofloxacin, moxafloxacin or gatifloxacin.

The compounds of general formula (I), whether or not in combination with another compound, may be administered by any suitable route, for example oral, rectal, nasal, bronchial (inhaled), topical (including eye drops, buccal and sublingual), vaginal or parenteral (including subcutaneous, intramuscular, intravenous and intradermal) administration and may be prepared by any methods well known in the art of pharmacy. Oral and parenteral administration are, however, preferred, with the oral route being particularly suitable as oral administration is more likely to ensure patient compliance.

The composition may be prepared by bringing into association the above defined active agent with the carrier. In general, the formulations are prepared by uniformly and intimately bringing into association the active agent with liquid carriers or finely divided solid carriers or both, and then if necessary shaping the product.

Formulations for oral administration in the present invention may be presented as: discrete units such as capsules, sachets or tablets each containing a predetermined amount of the active agent; as a powder or granules; as a solution or a suspension of the active agent in an aqueous liquid or a non-aqueous liquid; or as an oil-in-water liquid emulsion or a water in oil liquid emulsion; or as a bolus etc.

For compositions for oral administration (e.g. tablets and capsules), the term “acceptable carrier” includes vehicles such as common excipients e.g. binding agents, for example syrup, acacia, gelatin, sorbitol, tragacanth, polyvinylpyrrolidone (Povidone), methylcellulose, ethylcellulose, sodium carboxymethylcellulose, hydroxypropylmethylcellulose, sucrose and starch; fillers and carriers, for example corn starch, gelatin, lactose, sucrose, microcrystalline cellulose, kaolin, mannitol, dicalcium phosphate, sodium chloride and alginic acid; and lubricants such as magnesium stearate, sodium stearate and other metallic stearates, glycerol stearate stearic acid, silicone fluid, talc waxes, oils and colloidal silica. Flavouring agents such as peppermint, oil of wintergreen, cherry flavouring and the like can also be used. It may be desirable to add a colouring agent to make the dosage form readily identifiable. Tablets may also be coated by methods well known in the art.

A tablet may be made by compression or moulding, optionally with one or more accessory ingredients. Compressed tablets may be prepared by compressing in a suitable machine the active agent in a free flowing form such as a powder or granules, optionally mixed with a binder, lubricant, inert diluent, preservative, surface-active or dispersing agent. Moulded tablets may be made by moulding in a suitable machine a mixture of the powdered compound moistened with an inert liquid diluent. The tablets may optionally be coated or scored and may be formulated so as to provide slow or controlled release of the active agent.

Other formulations suitable for oral administration include lozenges comprising the active agent in a flavoured base, usually sucrose and acacia or tragacanth; pastilles comprising the active agent in an inert base such as gelatin and glycerin, or sucrose and acacia; and mouthwashes comprising the active agent in a suitable liquid carrier.

Parenteral formulations will generally be sterile.

The invention will now be described in more detail with reference to the following examples.

### EXAMPLE 1

### Preparation of Compounds of General Formula (I)

### 1. General Experimental

HPLC-UV-MS was performed on a Gilson 321 HPLC with detection performed by a Gilson 170 DAD and a Finnigan AQA mass spectrometer operating in electrospray ionisation mode. The HPLC column used is a Phenomenex Gemini C18 150×4.6 mm. Preparative HPLC was performed on a Gilson 321 with detection performed by a Gilson 170 DAD. Fractions were collected using a Gilson 215 fraction collector. The preparative HPLC column used is a Phenomenex Gemini C18 150×10 mm and the mobile phase is acetonitrile/water.

¹H NMR spectra were recorded on a Bruker instrument operating at 300 MHz. NMR spectra were obtained as CDCl₃ solutions (reported in ppm), using chloroform as the reference standard (7.26 ppm) or DMSO-d₆ (2.50 ppm). When peak multiplicities are reported, the following abbreviations are used s (singlet), d (doublet), t (triplet), m (multiplet), br (broadened), dd (doublet of doublets), dt (doublet of triplets), td (triplet of doublets), obsc. (obscured), app. (apparent). Coupling constants, when given, are reported in Hertz (Hz).

Column chromatography was performed either by flash chromatography (40-65 μm silica gel) or using an automated purification system (SP1™ Purification System from Biotage® or CombiFlash Companion from ISCO). Reactions in the microwave were done in an Initiator 8™ (Biotage) or in an Explorer 48 (CEM).

The abbreviations used are:

DMSO dimethylsulfoxide
HCl hydrochloric acid
MgSO₄ magnesium sulfate
NaOH sodium hydroxide
Na₂CO₃ sodium carbonate
NaHCO₃ sodium bicarbonate
THF tetrahydrofuran
DMF dimethylformamide
IMS industrial methylated spirits
TLC thin layer chromatography
Boc tert-butyloxycarbonyl
RT room temperature
DCM dichloromethane
TFA trifluoroacetic acid

### NMP N-methylpyrrolidinone

TBAF tetrabutylammonium fluoride
DMPU 1,3-Dimethyl-3,4,5,6-tetrahydro-2(1H)-pyrimidinone)
EtOAc ethyl acetate
NEt₃ triethylamine
MeCN acetonitrile
CuSO₄ copper sulphate
IPA isopropyl alcohol
NH₄Cl ammonium chloride
DPPA diphenyl phosphoryl azide

### 2. Commercial Compounds

All compounds below were purchased from Chembridge:
N-(1-Phenylethyl)-2-(piperidin-1-yl)quinazolin-4-amine (Compound 12)
N-(Furan-2-ylmethyl)-2-(piperidin-1-yl)quinazolin-4-amine (Compound 13)
N-(1-Phenylethyl)-2-(pyrrolidin-1-yl)quinazolin-4-amine (Compound 14)
N-(1-Phenylethyl)-2-(4-(phenylsulfonyl)piperazin-1-yl)quinazolin-4-amine (Compound 15)
N-(2′-Adamantyl)-2-morpholinoquinazolin-4-amine (Compound 16)
2-(4-(Methylsulfonyl)piperazin-1-yl)-N-(1-phenylethyl)quinazolin-4-amine (Compound 10)
N-Benzyl-2-morpholinoquinazolin-4-amine (Compound 5)
N-Benzyl-2-(4-methylpiperazin-1-yl)quinazolin-4-amine hydrochloride (Compound 6)
N-(1-Phenylethyl)-2-(piperazin-1-yl)quinazolin-4-amine (Compound 8)
2-Morpholino-N-(1-phenylethyl)quinazolin-4-amine (Compound 9)
N⁴-Benzyl-N²-phenylquinazoline-2,4-diamine (Compound 23)
N¹-(2-Morpholinoquinazolin-4-yl)-N²-(4-nitrophenyl)ethane-1,2-diamine (Compound 7)
N¹-(2-(4-Methylpiperazin-1-yl)quinazolin-4-yl)-N²-(4-nitrophenyl)ethane-1,2-diamine (Compound 11)

All compounds below were purchased from Cheshire Sciences:
N²,N⁴-Dibenzylquinazoline-2,4-diamine (Compound 18)
Cyclopentyl(4-(4-(1-phenylethylamino)quinazolin-2-yl)piperazin-1-yl)methanone (Compound 21)
N-Benzyl-2-(piperidin-1-yl)quinazolin-4-amine (Compound 4)

All compounds below were purchased from Princeton Biomolecular:
2-Morpholino-N-(1-[2′-adamantyl])quinazolin-4-amine (Compound 19)
N-Benzyl-1-methyl-6-(pyrrolidin-1-yl)-1H-pyrazolo[3,4-d]pyrimidin-4-amine (Compound 20)

All compounds below were purchased from Labotest:
Quinazoline-2,4-diamine (Comparative compound 2)

The following compound was purchased from Life Chemicals:
N-(4-Fluorophenyl)-2-(pyrrolidin-1-yl)pteridin-4-amine (Compound 185)

### 3. Methods for Preparing Compounds of General Formulae (VI) and (IV)

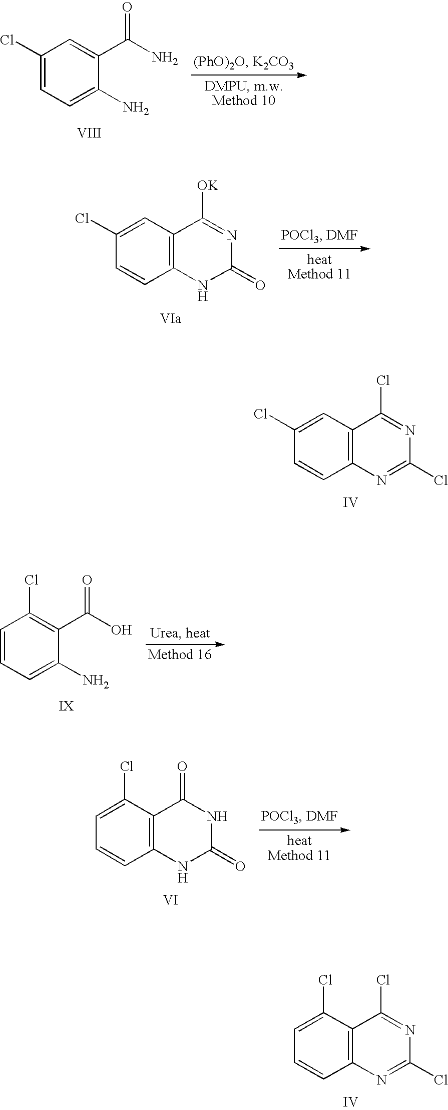

### Method 10

### Compounds of General Formula (VIa)

### 6-Chloroquinazoline-2,4(1H,3H)-dione monopotassium salt

2-Amino-5-chlorobenzamide (500 mg, 2.93 mmol), diphenylcarbonate (628 mg, 2.93 mmol) and K₂CO₃ (608 mg, 4.40 mmol) were suspended in DMPU (3 mL) and the mixture heated to 150° C. for 10 min under microwave irradiation. After cooling to ambient temperature, the suspension was poured into water, forming a precipitate. The mixture was filtered, and washed with EtOAc. The precipitate was heated in boiling EtOAc, filtered, and washed with cold EtOAc to give the title compound as an orange powder (594 mg, 86%).

¹H NMR (DMSO): 9.62 (1H, br s), 7.57 (1H, d, J 2.6), 7.22 (1H, dd, J 8.8 and 2.6) and 6.85 (1H, d, J 8.7).

### Method 11

### Compounds of General Formula (IV)

### 2,4,6-Trichloroquinazoline

A mixture of POCl₃ (10 mL) and DMF (4 drops) was stirred at ambient temperature for 30 min, prior to its addition to a flask containing 5-chloroquinazoline-2,4(1H,3H)-dione monopotassium salt (594 mg, 2.53 mmol). The mixture was heated to gentle reflux for 16 h. The resulting dark orange solution was cooled to ambient temperature and poured into ice water. A dark brown oil formed that on stirring formed a brown precipitate. The suspension was filtered and washed with copious water. Most of the precipitate was transferred to a round-bottomed flask, and the remainder washed off the sinter with THF (80 mL). The THF washings were added to the flask and concentrated. The residual solid was then dissolved in boiling IMS, filtered and the filtrate concentrated to afford the title compound as a light brown solid (132 mg, 22%).

¹H NMR (DMSO): 8.56 (1H, dd, J 2.4 and 0.5), 8.41 (1H, dd, J 9.0 and 2.3) and 8.30 (1H, dd, J 9.1 and 0.5).

The following compounds were prepared in a similar manner, purifying by crystallisation or column chromatography where necessary:

### 2,4,5-Trichloroquinazoline

¹H NMR (DMSO): 7.74 (1H, t, J 8.01), 7.57-7.55 (1H, m) and 7.54-7.53 (1H, m).

### 2,4-Dichloro-7-fluoroquinazoline

¹H NMR (DMSO): 8.43 (1H, dd, J 9.2 and 5.8), 7.95 (1H, dd, J 9.6 and 2.5) and 7.88-7.80 (1H, m).

### Method 16

### Compounds of General Formula (VI)

### 5-Chloroquinazoline-2,4(1H,3H)-dione

2-Amino-6-chlorobenzoic acid (300 mg, 1.75 mmol) and urea (1.1 g) were mixed in a Radley's carousel tube and heated to 170° C. for 18 h, during which time a melt formed. The melt was cooled to ambient temperature and suspended in water by sonication. The solid was collected by filtration, transferred to a conical flask and dissolved in hot 1N NaOH. The product was triturated with glacial acetic acid, filtered and washed with water. After drying under vacuum at 50° C. for 18 h the product was obtained as a light brown solid (351 mg, 100% crude yield). This material was carried forward without further purification. ¹H NMR (DMSO): 11.10 (2H, br m), 7.54 (1H, t, J 8.1), 7.19 (1H, dd, J 7.8 and 1.0) and 7.12 (1H, dd, J 8.3 and 1.1).

The following compound was prepared in a similar manner, purifying by crystallisation or column chromatography where necessary:

### 7-Fluoroquinazoline-2,4(1H,3H)-dione

¹H NMR (DMSO): 11.31 (2H, br s), 7.94 (1H, dd, J 8.8 and 6.2), 7.02 (1H, td, J 8.8 and 2.5) and 6.90 (1H, dd, J 10.0 and 2.5).

### 4. Methods 1 to 4

### Quinazoline S_{N}Ar for the Preparation of Compounds of General Formulae (II) and (I)

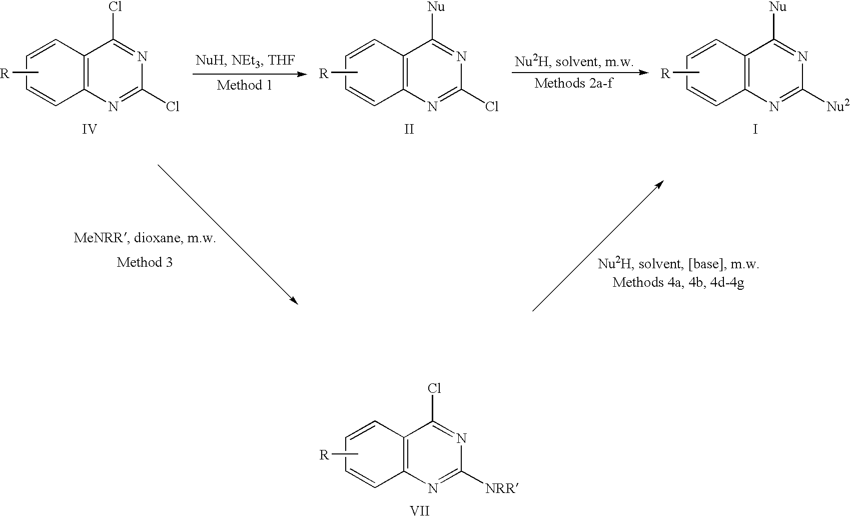

### Method 1

### Compounds of General Formula (II)

### N-Benzyl-2-chloro-6,7-dimethoxyquinazolin-4-amine

2,4-Dichloro-6,7-dimethoxyquinazoline (100 mg, 0.39 mmol) was dissolved in THF (1 mL). NEt₃ (65 □L, 0.46 mmol) was added, followed by benzylamine (44 □L, 0.41 mmol). The mixture was stirred at room temperature until TLC analysis indicated no starting material remained. The mixture was concentrated in vacuo, redissolved in EtOAc and washed with saturated aqueous K₂CO₃ and brine. The organic layer was separated, dried (MgSO₄), filtered and concentrated to give the title compound as a yellow solid (92 mg, 72%). This material was carried forward to Method 2a.

¹H NMR (DMSO): 8.88 (1H, t, J 5.7), 7.69 (1H, s), 7.41-7.23 (5H, m), 7.09 (1H, s), 4.74 (2H, d, J 5.8), 3.89 (3H, s) and 3.87 (3H, s).

The following compounds were prepared in a similar manner, purifying by crystallisation or column chromatography where necessary:

### N-Benzyl-2-chloroquinazolin-4-amine

¹H NMR (DMSO): 9.29 (1H, t, J 5.6), 8.32 (1H, dd, J 8.3 and 0.8), 7.82 (1H, ddd, J 8.3, 7.0 and 1.3), 7.64 (1H, dd, J 8.3 and 0.8), 7.55 (1H, ddd, J 8.3, 7.0 and 1.2), 7.40-7.23 (5H, m) and 4.76 (2H, d, J 5.9).

### 2-Chloro-4-(piperidin-1-yl)quinazoline

¹H NMR (DMSO): 7.99-7.95 (1H, m), 7.81 (1H, ddd, J 8.4, 7.0 and 1.3), 7.69 (1H, dd, J 8.4 and 1.00), 7.52 (1H, ddd, J 8.3, 6.9 and 1.3), 3.82-3.74 (4H, m) and 1.74-1.68 (6H, m).

### 2-Chloroquinazolin-4-amine

¹H NMR (DMSO): 8.39 (2H, br s), 8.29 (1H, dd, J, 8.2 and 0.8), 7.87 (1H, ddd, J, 8.3, 7.0 and 1.3), 7.69-7.66 (1H, m) and 7.58 (1H, ddd, J 8.1, 7.0 and 1.1).

### 2-Chloro-N-(1-adamantyl)quinazolin-4-amine

LCMS RT=8.89 min, MH⁺ 314.2; ¹H NMR (DMSO): 8.42 (1H, d, J 8.5 and 0.8), 7.77 (1H, ddd, J 8.4, 6.9 and 1.3), 7.60-7.56 (2H, m), 7.50 (1H, ddd, J 8.3, 7.0 and 1.3), 2.28-2.23 (6H, m), 2.14-2.08 (3H, br m) and 1.71-1.67 (6H, m).

### 2-Chloro-N-(2-adamantyl)quinazolin-4-amine

LCMS RT=8.61 min, MH⁺ 314.3; ¹H NMR (DMSO): 8.56 (1H, d, J 7.8), 7.83-7.78 (2H, m), 7.63-7.60 (1H, m), 7.56-7.51 (1H, m), 4.31-4.25 (1H, m), 2.21-2.11 (4H, m), 1.92-1.81 (6H, m), 1.77-1.72 (2H, m) and 1.61-1.52 (2H, m).

### N-Benzhydryl-2-chloroquinazolin-4-amine

¹H NMR (DMSO): 9.34 (1H, d, J 8.5), 8.60 (1H, dd, J 8.4 and 0.8), 7.83 (1H, ddd, J 8.4, 7.0 and 1.3), 7.64 (1H, dd, J 8.4 and 0.9), 7.56 (1H, ddd, J 8.3, 7.0 and 1.3), 7.45-7.27 (10H, m) and 6.78 (1H, d, J 8.5).

### 2-Chloro-N-(2-phenylpropan-2-yl)quinazolin-4-amine

¹H NMR (DMSO): 8.57 (1H, d, J 7.7), 8.46 (1H, br s, NH), 7.80 (1H, ddd, J 8.2, 6.9 and 1.2), 7.61-7.53 (2H, m), 7.41-7.35 (2H, m), 7.30-7.22 (2H, m), 7.16 (1H, ddd, J 8.4, 6.4 and 1.2) and 1.83 (6H, s).

### N-Benzyl-2,6-dichloroquinazolin-4-amine

¹H NMR (DMSO): 9.36 (1H, t, J 5.9), 8.50 (1H, d, J 2.3), 7.84 (1H, dd, J 8.9 and 2.3), 7.66 (1H, d, J 8.9), 7.40-7.24 (5H, m) and 6.87 (2H, br s).

### 2-Chloro-N-(4-chlorobenzyl)quinazolin-4-amine

LCMS RT=6.57 min, MH⁺ 355.2; ¹H NMR (DMSO): 9.33-9.27 (1H, m), 8.30 (1H, d, J 8.0), 7.85-7.79 (1H, m), 7.66-7.61 (1H, m), 7.59-7.53 (1H, m), 7.41-7.35 (4H, m) and 4.74 (2H, d, J 6.0).

### 2-Chloro-N-(4-(trifluoromethyl)benzyl)quinazolin-4-amine

LCMS RT=6.61 min, MH⁺ 338.2; ¹H NMR (DMSO): 9.41-9.35 (1H, m), 8.32 (1H, dd, J 8.4 and 0.8), 7.84 (1H, ddd, J 8.3, 7.0 and 1.3), 7.73-7.69 (2H, m), 7.66 (1H, dd, J 8.4 and 0.8), 7.61-7.55 (3H, m) and 4.84 (2H, d, J 5.8).

### 2-Chloro-N-(2-p-tolylpropan-2-yl)quinazolin-4-amine

¹H NMR (DMSO): 8.57 (1H, d, J 7.8), 8.40 (1H, br s, NH), 7.80 (1H, ddd, J 8.4, 6.8 and 1.3), 7.61-7.52 (2H, m), 7.26 (2H, d, J 8.4), 7.06 (2H, d, J 7.9), 2.24 (3H, s) and 1.81 (6H, s).

### 2-Chloro-N-(1-(4-fluorophenyl)-2-methylpropan-2-yl)quinazolin-4-amine

LCMS RT=7.49 min, MH⁺ 330.2 ¹H NMR (DMSO): 8.36 (1H, d, J 8.3), 7.80 (1H, t, J 7.8), 7.64 (1H, d, J 8.3), 7.58-7.45 (2H, br m), 7.04 (4H, d, J 8.1), 3.32 (2H, br s) and 1.48 (6H, s).

### 2-Chloro-N-(4-(dimethylamino)benzyl)quinazolin-4-amine

¹H NMR (DMSO): 9.18 (1H, t, J 5.8), 8.29 (1H, dd, J 8.3 and 0.8), 7.79 (1H, ddd, J 8.4, 7.0 and 1.3), 7.62 (1H, 8.4 and 0.8), 7.52 (1H, ddd, J 8.2, 7.0 and 1.2), 7.24-7.19 (2H, m), 6.71-6.67 (2H, m), 4.62 (2H, d, J 5.7) and 2.85 (6H, s).

### 2-Chloro-N-(2-(4-fluorophenyl)propan-2-yl)quinazolin-4-amine

¹H NMR (DMSO): 8.56 (1H, d, J 8.3), 8.46 (1H, br s), 7.83-7.77 (1H, m), 7.61-7.53 (2H, m), 7.43-7.37 (2H, m), 7.12-7.03 (2H, m) and 1.82 (6H, s).

### 2-Chloro-N-(3-phenylpentan-3-yl)quinazolin-4-amine

¹H NMR (CDCl₃): 7.81-7.72 (3H, m), 7.53-7.25 (6H, m), 6.06 (1H, br s), 2.55-2.32 (4H, m) and 0.83-0.75 (6H, m).

### 2-Chloro-N-(1-phenylcyclopropyl)quinazolin-4-amine

¹H NMR (DMSO): 9.42 (1H, br s), 8.40 (1H, d, J 8.4), 7.85-7.80 (1H, m), 7.65-7.61 (1H, m), 7.59-7.54 (1H, m), 7.30-7.21 (4H, m), 7.19-7.13 (1H, m) and 1.40 (4H, s).

### 2-Chloro-N-(1-phenylcyclohexyl)quinazolin-4-amine

¹H NMR (DMSO): 8.64-8.60 (1H, m), 8.10 (1H, br s), 7.84-7.78 (1H, m), 7.61-7.55 (2H, m), 7.45-7.41 (2H, m), 7.31-7.24 (2H, m), 7.19-7.13 (1H, m), 2.90-2.80 (2H, br d), 1.90-1.77 (2H, m), 1.70-1.54 (5H, m) and 1.42-1.49 (1H, m).

### N-Benzyl-2,5-dichloroquinazolin-4-amine

¹H NMR (DMSO): 8.92 (1H, t, J 5.7), 7.76 (1H, t, J 8.0), 7.64-7.62 (1H, m), 7.61-7.59 (1H, m), 7.44-7.40 (2H, m), 7.37-7.32 (2H, m), 7.29-7.23 (1H, m) and 4.80 (2H, d, J 5.9).

### 2-Chloro-N-(4-fluorobenzyl)-6,7-dimethoxyquinazolin-4-amine

¹H NMR (DMSO): 8.88 (1H, br s), 7.67 (1H, s), 7.46-7.38 (2H, br m), 7.18 (2H, t, J 9.0), 7.10 (1H, s), 4.72 (2H, d, J 5.6), 3.89 (3H, s), 3.87 (3H, s).

### 2-Chloro-N-((5-methylfuran-2-yl)methyl)quinazolin-4-amine

¹H NMR (DMSO): 9.19 (1H, t, J 5.5), 8.30 (1H, dd, J 8.4 and 1.0), 7.81 (1H, ddd, J 8.4, 7.0 and 1.3), 7.63 (1H, dd, J 8.4 and 0.9), 7.54 (1H, ddd, J 8.3, 7.0 and 1.3), 6.23 (1H, br. d, J 3.1), 6.02-6.00 (1H, m), 4.67 (2H, d, J 5.5) and 2.23 (3H, s).

### 2-Chloro-N-((5-(trifluoromethyl)furan-2-yl)methyl)quinazolin-4-amine

¹H NMR (DMSO): 9.32 (1H, t, J 5.5), 8.29 (1H, dd, J 8.4 and 0.8), 7.84 (1H, ddd, J 8.4, 7.0 and 1.4), 7.66 (1H, dd, J 8.5 and 0.8), 7.57 (1H, ddd, J 8.3, 7.0 and 1.3), 7.20-7.16 (1H, m), 6.61-6.58 (1H, m) and 4.80 (2H, d, J 5.5).

### 2-Chloro-N-(4-(trifluoromethoxy)benzyl)quinazolin-4-amine

¹H NMR (DMSO): 9.38 (1H, br s), 8.37 (1H, d, J 8.4), 7.88 (1H, td, J 7.7 and 1.3), 7.71 (1H, d, J 8.5), 7.66-7.53 (3H, br s), 7.40 (2H, d, J 8.5), 4.84 (2H, d, J 6.0).

### 4-((2-Chloroquinazolin-4-ylamino)methyl)phenol

¹H NMR (DMSO): 9.40 (1H, s), 9.25 (1H, br s), 8.35 (1H, d, J 8.3), 7.86 (1H, t, J 7.6), 7.68 (1H, d, J 8.3), 7.59 (1H, Y, J 7.6), 7.24 (2H, d, J 8.4), 6.78 (2H, d, J 8.3), 4.69 (2H, d, J 5.8).

### 2-Chloro-N-(4-fluorobenzyl)thieno[3,2-d]pyrimidin-4-amine

¹H NMR (DMSO): 8.93 (1H, br s), 8.19 (1H, d, J 5.4), 7.43-7.33 (3H, br m), 7.16 (2H, t, J 8.8), 4.66 (2H, d, J 5.8).

### 2-Chloro-N-(4-fluorobenzyl)pyrido[2,3-d]pyrimidin-4-amine

LCMS RT=1.81 min, MH⁺ 289.0; ¹H NMR (DMSO): 9.56 (1H, br s), 8.99 (1H, dd, J 4.5 and 1.8), 8.75 (1H, dd, J 8.3 and 1.8), 7.59 (1H, dd, J 8.3 and 4.4), 7.47-7.39 (2H, br m), 7.22-7.12 (2H, br m) and 4.74 (2H, d, J 4.6).

### 2,5-Dichloro-N-(4-fluorobenzyl)quinazolin-4-amine

¹H NMR (DMSO): 8.94 (1H, t, J 5.7), 7.77 (1H, t, J 8.0), 7.65-7.61 (2H, m), 7.52-7.45 (2H, m), 7.22-7.14 (2H, m) and 4.79 (2H, d, J 5.8).

### tert-Butyl 2-chloro-4-(4-fluorobenzylamino)-5,6-dihydropyrido[3,4-d]pyrimidine-7(8H)-carboxylate

¹H NMR (CDCl₃): 7.35-7.28 (2H, m), 7.08-7.00 (2H, m), 4.97 (1H, br s), 4.67 (2H, d, J 5.5), 4.45 (2H, br s), 3.69 (2H, t, J 5.8), 2.40-2.34 (2H, m) and 1.46 (9H, s).

### 2-Chloro-4-(3,4-dihydroisoquinolin-2(1H)-yl)quinazoline

¹H NMR (DMSO): 8.18 (1H, dd, J 8.5 and 0.9), 7.85 (1H, ddd, J 8.4, 6.9 and 1.3), 7.71 (1H, dd, J 8.4 and 1.0), 7.56 (1H, ddd, J 8.4, 7.0 and 1.4), 7.33-7.14 (4H, m), 4.96 (2H, br s), 4.06 (2H, t, J 5.9) and 3.10 (2H, t, J 5.8).

### 2-Chloro-N-(thiophen-2-ylmethyl)quinazolin-4-amine

¹H NMR (DMSO): 9.41-9.35 (1H, br t), 8.25 (1H, dd, J 8.4 and 0.9), 7.81 (1H, ddd, J 8.3, 7.0 and 1.3), 7.65 (1H, dd, J 8.4 and 0.8), 7.55 (1H, ddd, J 8.2, 7.0 and 1.2), 7.40 (1H, dd, J 5.1 and 1.2), 7.11 (1H, dd, J 3.4 and 1.0), 6.98 (1H, dd, J 5.1 and 3.5) and 4.89 (2H, d, J 5.9).

### 2-Chloro-4-(6,7-dimethoxy-3,4-dihydroisoquinolin-2(1H)-yl)quinazoline

¹H NMR (DMSO): 8.18-8.13 (1H, m), 7.84 (1H, ddd, J 8.3, 7.0 and 1.3), 7.71 (1H, d, J 8.4 and 1.1), 7.56 (1H, ddd, J 8.4, 7.0 and 1.3), 6.92 (1H, s), 6.83 (1H, s), 4.90 (2H, br s), 4.06-3.99 (2H, m), 3.74 (3H, s), 3.72 (3H, s) and 3.04-2.97 (2H, m).

### 2-Chloro-N-(1-(4-fluorophenyl)cyclopropyl)quinazolin-4-amine

¹H NMR (DMSO): 9.44 (1H, br s), 8.37 (1H, d, J 8.6), 7.82 (1H, ddd, J 8.4, 7.0 and 1.3), 7.66-7.61 (1H, m), 7.59-7.53 (1H, m), 7.35-7.28 (2H, m), 7.14-7.05 (2H, m) and 1.37 (4H, br s).

### 2-Chloro-N-(4-fluorobenzyl)quinazolin-4-amine

¹H NMR (DMSO): 9.38 (1H, t, J 5.8), 8.32 (1H, dd, J 8.3 and 0.7), 7.83 (1H, ddd, J 8.3, 7.0 and 1.3), 7.74-7.53 (6H, m) and 4.84 (2H, d, J 5.7).

### 2-Chloro-4-(4,4-difluoropiperidin-1-yl)quinazoline

¹H NMR (DMSO): 8.06 (1H, dd, J 8.5 and 0.8), 7.86 (1H, ddd, J 8.4, 7.0 and 1.3), 7.75 (1H, dd, J 8.3 and 1.1), 7.57 (1H, ddd, J 8.4, 7.0 and 1.4), 3.93-3.85 (4H, m) and 2.30-2.13 (4H, m).

### 2-Chloro-7-fluoro-N-(4-fluorobenzyl)quinazolin-4-amine

¹H NMR (DMSO): 9.36 (1H, t, J 5.8), 8.39 (1H, dd, J 9.2 and 5.9), 7.52-7.38 (4H, m), 7.22-7.12 (2H, m) and 4.72 (2H, d, J 5.7).

### 2-Chloro-N-(1-(4-chlorophenyl)cyclopropyl)quinazolin-4-amine

¹H NMR (DMSO): 9.44 (1H, br s), 8.38 (1H, dd, J 8.3 and 0.7), 7.83 (1H, ddd, J 8.4, 7.0 and 1.3), 7.64 (1H, dd, J 8.4 and 0.8), 7.57 (1H, ddd, J 8.3, 7.0 and 1.2), 7.35-7.22 (4H, m) and 1.40 (4H, br s).

### Method 2a

### Compounds of General Formula (I)

### 2-(Piperidin-1-yl)quinazolin-4-amine (Comparative Compound 1)

2-Chloroquinazolin-4-amine (150 mg, 0.84 mmol) was dissolved in IMS (Solvent S, 3 mL). Piperidine (248 □L, 2.5 mmol, 3 eq. E) was added and the mixture was heated to 150° C. (Temperature K) for 5 min (Time T) under microwave irradiation. The mixture was concentrated in vacuo, diluted with EtOAc and extracted with EtOAc (2×20 mL) from aqueous K₂CO₃ or NaHCO₃. The organic layer was washed with brine, dried (MgSO₄), filtered and concentrated in vacuo to give the title compound as a yellow solid (172 mg, 90%).

¹H NMR (CDCl₃): 7.55-7.42 (3H, m), 7.04 (1H, ddd, J 8.1, 6.7 and 1.4, 5.24 (2H, br s), 3.86-3.81 (4H, m) and 1.68-1.56 (6H, m).

The following compounds were prepared in a similar manner, purifying by crystallisation or column chromatography where necessary:

### N-Benzyl-6,7-dimethoxy-2-morpholinoquinazolin-4-amine (Compound 17)

LCMS RT=5.99 min, MH⁺ 381.2; ¹H NMR (DMSO): 8.30 (1 h, br s), 7.50 (1H, s), 7.38-7.20 (5H, m), 6.75 (1H, s), 4.69 (2H, d, J 5.6), 3.82 (3H, s) and 3.80 (3H, s).

### N-(1′-Adamantyl)-2-(piperidin-1-yl)quinazolin-4-amine (Compound 59)

LCMS RT=5.33 min, MH⁺ 363.2; ¹H NMR (DMSO): 8.07 (1H, dd, J 8.3 and 1.1), 7.45 (1H, ddd, J 8.3, 6.9 and 1.3), 7.21 (1H, dd, J 8.3 and 0.8), 7.02-6.97 (1H, m), 6.77 (1H, br s), 3.79-3.76 (4H, m), 2.27-2.24 (6H, m), 2.14-2.06 (3H, br m), 1.72-1.66 (6H, br m), 1.66-1.58 (2H, m) and 1.56-1.47 (4H, m).

### N-(2′-Adamantyl)-2-(piperidin-1-yl)quinazolin-4-amine (Compound 60)

LCMS RT=5.33 min, MH⁺ 363.2; ¹H NMR (DMSO): 8.20 (1H, dd, J 8.3 and 1.0), 7.47 (1H, ddd, J 8.4, 6.9 and 1.4), 7.23 (1H, dd, J 8.5 and 0.9), 7.09-7.06 (1H, m), 7.02 (1H, ddd, J 8.1, 6.9 and 1.2), 4.26-4.20 (1H, m), 3.79-3.72 (4H, m), 2.23-2.09 (4H, m), 1.92-1.79 (6H, m), 1.77-1.72 (2H, m) and 1.66-1.44 (8H, m).

### N-(1′-Adamantyl)-2-(morpholino)quinazolin-4-amine (Compound 61)

LCMS RT=4.71 min, MH⁺ 365.3; ¹H NMR (DMSO): 8.11 (1H, dd, J 8.3 and 0.9), 7.49 (1H, ddd, J 8.4, 6.9 and 1.3), 7.25 (1H, dd, J 8.4 and 0.9), 7.05 (1H, ddd, J 8.2, 6.9 and 1.2), 6.86 (1H, br s), 3.75-3.64 (8H, m), 2.28-2.21 (6H, m), 2.14-2.07 (3H, br m) and 1.72-1.66 (6H, br m).

### N-Benzhydryl-2-(piperidin-1-yl)quinazolin-4-amine (Compound 50)

LCMS RT=9.55 min, MH⁺ 395.3; ¹H NMR (DMSO): 8.50 (1H, d, J 7.8), 8.27 (1H, dd, J 8.2 and 0.7), 7.49 (1H, ddd, J 8.4, 6.9 and 1.3), 7.43-7.22 (11H, m), 7.03 (1H, ddd, J 8.1, 6.9 and 1.2), 6.65 (1H, d, J 7.7), 3.73-3.69 (4H, m), 1.62-1.52 (2H, m) and 1.44-1.35 (4H, m).

### N-(4-(Dimethylamino)benzyl)-2-(piperidin-1-yl)quinazolin-4-amine (Compound 62)

LCMS RT=8.51 min, MH⁺ 362.3; ¹H NMR (DMSO): 8.39-8.32 (1H, m), 7.97 (1H, dd, J 8.2 and 0.9), 7.46 (1H, ddd, J 8.4, 6.9 and 1.4), 7.24-7.19 (2H, m), 7.01 (1H, ddd, J 8.1, 6.9 and 1.2), 6.69-6.63 (2H, m), 4.56 (2H, d, J 5.9), 3.80-3.74 (4H, m), 2.83 (6H, s), 1.64-1.56 (2H, m) and 1.53-1.43 (4H, m).

### N-Benzyl-6,7-dimethoxy-2-(piperidin-1-yl)quinazolin-4-amine (Compound 70)

LCMS RT=8.22 min, MH⁺ 379.2; ¹H NMR (DMSO): 8.20 (1H, t, J 5.9), 7.46 (1H, s), 7.40-7.36 (2H, m), 7.33-7.27 (2H, m), 7.24-7.18 (1H, m), 6.71 (1H, s), 4.67 (2H, d, J 5.7), 3.82 (3H, s), 3.79 (3H, s), 3.71-3.66 (4H, m), 1.61-1.53 (2H, m) and 1.47-1.39 (4H, m).

### N-Benzyl-6-chloro-2-(piperidin-1-yl)quinazolin-4-amine (Compound 71)

LCMS RT=9.32 min, MH⁺ 353.3; ¹H NMR (DMSO): 8.61 (1H, t, J 5.7), 8.15 (1H, d, J 2.5), 7.47 (1H, dd, J 8.9 and 2.4), 7.39-7.28 (3H, m), 7.25-7.19 (2H, m), 4.65 (2H, d, J 5.9), 3.73-3.70 (4H, m), 1.62-1.54 (2H, m) and 1.47-1.38 (4H, m).

### N-Benzyl-2-(1,4-oxazepan-4-yl)quinazolin-4-amine (Compound 52)

LCMS RT=6.73 min, MH⁺ 335.3; ¹H NMR (DMSO): 8.57 (1H, t, J 6.1, NH), 8.03 (1H, dd, J 8.2 and 0.9), 7.49 (1H, ddd, J 8.3, 6.9 and 1.4), 7.37-7.17 (6H, m), 7.05 (1H, ddd, J 8.1, 6.9 and 1.1), 4.67 (2H, d, J 5.7), 3.83-3.74 (4H, m), 3.64-3.46 (4H, m) and 1.85-1.65 (2H, m).

### N-(2-Phenylpropan-2-yl)-2-(piperidin-1-yl)quinazolin-4-amine (Compound 53)

LCMS RT=9.58 min, MH⁺ 347.3; ¹H NMR (DMSO): 8.23 (1H, dd, J 8.2 and 1.0), 7.68 (1H, br s), 7.46 (1H, ddd, J 8.2, 6.9 and 1.4), 7.36 (2H, m), 7.25-7.17 (3H, m), 7.11 (1H, ddd, J 8.4, 6.4 and 1.2), 7.05 (1H, ddd, J 8.2, 7.0 and 1.2), 1.76 (6H, s), 1.50-1.41 (4H, m) and 1.21-1.11 (4H, m).

### tert-Butyl 4-(4-(benzylamino)quinazolin-2-yl)-1,4-diazepane-1-carboxylate (Compound 55)

LCMS RT=8.32 min, MH⁺ 434.3; ¹H NMR (DMSO): 8.61-8.49 (1H, m, NH), 8.02 (1H, d, J 7.9), 7.48 (1H, ddd, J 8.3, 7.0 and 1.3), 7.39-7.17 (6H, m), 7.05 (1H, dd, J 7.5 and 6.9), 4.68 (2H, d, J 5.2), 3.84-3.58 (4H, m), 3.40-3.25 (2H, m), 3.23-3.07 (2H, m), 1.95-1.50 (2H, m) and 1.35-1.15 (9H, m).

### N⁴-Benzyl-N²-propylquinazoline-2,4-diamine (Compound 67)

[no LCMS] MH⁺ 293.2; ¹H NMR (DMSO): 8.38 (1H, br s, NH), 7.99 (1H, dd, J 8.3 and 1.0), 7.46 (1H, ddd, J 8.3, 7.1 and 1.4), 7.38-7.27 (4H, m), 7.25-7.18 (2H, m), 7.00 (1H, ddd, J 8.1, 6.9 and 1.0), 6.48 (1H, br s, NH), 4.71 (2H, d, J 5.8), 3.26-3.16 (2H, m), 1.57-1.39 (2H, m) and 0.85 (3H, t, J 7.4).

### N-(4-Chlorobenzyl)-2-morpholinoquinazolin-4-amine (Compound 74)

LCMS RT=4.55 min, MH⁺ 355.2; ¹H NMR (DMSO): 8.63 (1H, t, J 5.9), 8.03 (1H, dd, J 8.3 and 1.2), 7.52 (1H, ddd, J 8.4, 6.9 and 1.4), 7.41-7.34 (4H, m), 7.28 (1H, dd, J 8.4 and 0.8), 7.10 (1H, ddd, J 8.1, 7.0 and 1.2), 4.67 (2H, d, J 5.9), 3.70-3.63 (4H, m) and 3.61-3.55 (4H, m).

### N-(4-Chlorobenzyl)-2-(pyrrolidin-1-yl)quinazolin-4-amine (Compound 75)

LCMS RT=4.63 min, MH⁺ 339.3; ¹H NMR (DMSO): 8.51 (1H, t, J 6.1), 7.98 (1H, d, J 8.2 and 1.0), 7.47 (1H, ddd, J 8.4, 6.9 and 1.4), 7.42-7.33 (4H, m), 7.25 (1H, dd, J 8.4 and 0.8), 7.01 (1H, ddd, J 8.1, 6.9 and 1.2), 4.67 (2H, d, J 5.9), 3.49-3.42 (4H, m) and 1.90-1.83 (4H, m).

### 2-Morpholino-N-(4-(trifluoromethyl)benzyl)quinazolin-4-amine (Compound 77)

LCMS RT=4.57 min, MH⁺ 389.2; ¹H NMR (DMSO): 8.71 (1H, t, J 5.7), 8.05 (1H, dd, J 8.3 and 1.0), 7.68 (2H, app. d, J 8.1), 7.60-7.50 (3H, m), 7.29 (1H, dd, J 8.5 and 0.9), 7.11 (1H, ddd, J 8.1, 7.0 and 1.1), 4.76 (2H, d, J 5.5), 3.66-3.60 (4H, m) and 3.57-3.52 (4H, m).

### 2-(Pyrrolidin-1-yl)-N-(4-(trifluoromethyl)benzyl)quinazolin-4-amine (Compound 78)

LCMS RT=4.67 min, MH⁺ 373.2; ¹H NMR (DMSO): 8.58 (1H, t, J 5.9), 8.00 (1H, dd, J 8.2 and 1.0), 7.68 (2H, app. d, J 8.1), 7.59 (2H, app. d, J 8.2), 7.48 (1H, ddd, J 8.4, 6.9 and 1.5), 7.26 (1H, dd, J 8.5 and 0.8), 7.03 (1H, ddd, J 8.1, 6.9 and 1.2), 4.77 (2H, d, J 5.9), 3.47-3.41 (4H, m) and 1.89-1.81 (4H, m).

### N-(2-Phenylpropan-2-yl)-2-(pyrrolidin-1-yl)quinazolin-4-amine (Compound 91)

LCMS RT=4.65 min; MH⁺=333.3; ¹H NMR (DMSO): 8.23 (1H, d, J 7.1), 7.66 (1H, br s), 7.46 (1H, ddd, J 8.4, 7.0 and 1.4), 7.38 (2H, m), 7.26-7.17 (3H, m), 7.11 (1H, ddd, J 8.4, 6.9 and 1.3), 7.03 (1H, ddd, J 8.2, 7.0 and 1.2), 3.25-2.85 (4H, br s), 1.79 (6H, s) and 1.71 (4H, m).

### 2-(Piperidin-1-yl)-N-(2-p-tolylpropan-2-yl)quinazolin-4-amine (Compound 102)

LCMS RT=4.66 min; MH⁺=361.3; ¹H NMR (DMSO): 8.20 (1H, d, J 7.4), 7.62 (1H, br s), 7.46 (1H, ddd, J 8.3, 7.0 and 1.3), 7.23 (2H, d, J 8.1), 7.18 (1H, J 7.6), 7.07-6.98 (3H, m), 3.57-3.30 (4H, obscured), 2.21 (3H, s), 1.73 (6H, s), 1.53-1.40 (2H, m) and 1.24-1.10 (4H, m).

### 2-(Pyrrolidin-1-yl)-N-(2-p-tolylpropan-2-yl)quinazolin-4-amine (Compound 103)

LCMS RT=4.63 min; MH⁺=347.3; ¹H NMR (DMSO): 8.21 (1H, d, J 7.4), 7.60 (1H, br s), 7.45 (1H, ddd, J 8.2, 7.9 and 1.3), 7.26 (2H, d, J 8.3), 7.20 (1H, J 8.3), 7.06-6.98 (3H, m), 3.27-2.96 (4H, br s), 2.22 (3H, s), 1.78 (6H, s) and 1.76-1.69 (4H, m).

### N-(1-(4-Fluorophenyl)-2-methylpropan-2-yl)-2-(piperidin-1-yl)quinazolin-4-amine (Compound 108)

LCMS RT=4.69 min, MH⁺=379.2 ¹H NMR (DMSO): 8.01 (1H, d, J 8.5), 7.47 (1H, td, J 7.7 and 1.4), 7.25 (1H, dd, J 8.4 and 1.0), 7.07-6.94 (5H, br m), 6.69 (1H, br s), 3.87-3.78 (4H, br m), 1.69-1.59 (2H, br m), 1.59-1.48 (4H, br m) and 1.48-1.42 (6H, s).

### N-Benzyl-5-chloro-2-(piperidin-1-yl)quinazolin-4-amine (Compound 107)

LCMS RT=4.64 min, MH⁺ 353.2 ¹H NMR (DMSO): 8.40 (1H, br m), 7.53-7.44 (3H, br m), 7.44-7.35 (2H, br m), 7.34-7.25 (2H, br m), 7.16 (1H, dd, J 7.6 and 1.3), 4.81 (2H, d, J 5.9), 3.76 (4H, t, J 5.5), 1.72-1.60 (2H, br m) and 1.53-1.42 (4H, br m).

### N⁴-Benzyl-N²-(2,3-dihydro-1H-inden-2-yl)quinazoline-2,4-diamine (Compound 131)

LCMS RT=4.69 min, MH⁺ 367.2; ¹H NMR (DMSO): 12.10 (1H, br s), 10.28 (1H, br s), 8.42-8.24 (1H, br m), 7.81 (1H, t, J 7.4), 7.52-7.12 (11H, br m), 4.88-4.69 (3H, br m), and 3.26-2.79 (4H, br m—obscured by NMR solvent signal).

### N-Benzyl-2-(isoindolin-2-yl)quinazolin-4-amine (Compound 130)

LCMS RT=4.68 min, MH⁺ 353.2; ¹H NMR (DMSO): 8.62 (1H, t, J 6.0), 8.07 (1H, d, J 8.0), 7.53 (1H, t, J 7.7), 7.46 (2H, d, J 7.4), 7.43-7.37 (2H, br m), 7.37-7.27 (5H, br m), 7.26-7.19 (1H, br m), 7.08 (1H, t, J 7.5) and 4.85-4.76 (6H, br m).

### N-(1-Phenylcyclohexyl)-2-(pyrrolidin-1-yl)quinazolin-4-amine (Compound 125)

LCMS RT=4.72 min, MH⁺ 373.3; ¹H NMR (DMSO): 8.27 (1H, d, J 8.0), 7.50-7.41 (3H, br m), 7.32 (1H, s), 7.27-7.17 (3H, br m), 7.11 (1H, t, J 7.1), 7.04 (1H, t, J 7.5), 3.26-2.96 (4H, br m—obscured by water signal), 2.94-2.77 (2H, br m) and 1.85-1.50 (12H, br m).

### N-(3-Phenylpentan-3-yl)-2-(pyrrolidin-1-yl)quinazolin-4-amine (Compound 124)

LCMS RT=4.67 min, MH⁺ 361.3; NMR (CDCl₃): 7.51 (1H, d, J 8.2), 7.48-7.39 (2H, br m), 7.34-7.26 (2H, br m), 7.20 (2H, t, J 7.3—partially obscured by NMR solvent signal), 7.10 (1H, t, J 7.1), 7.04-6.94 (1H, br m), 5.56 (1H, br s), 3.36-2.98 (4H, br m), 2.44-2.28 (2H, br m), 2.17 (2H, br m), 1.81-1.48 (4H, br m) and 0.71 (6H, t, J 7.4).

### N-(1-Phenylcyclopropyl)-2-(pyrrolidin-1-yl)quinazolin-4-amine (Compound 123)

LCMS RT=4.00 min, MH⁺ 331.3; ¹H NMR (DMSO): 8.71 (1H, br s), 8.15 (1H, d, J 8.3), 7.55 (1H, td, J 7.6 and 1.2), 7.41-7.27 (5H, br m), 7.19 (1H, t, J 7.0), 7.09 (1H, td, J 7.4 and 1.3), 3.41-3.36 (4H, br m—obscured by water signal), 1.94-1.83 (4H, br m) and 1.43-1.34 (4H, d, br m).

### N-(1-Phenylcyclopropyl)-2-(piperidin-1-yl)quinazolin-4-amine (Compound 122)

LCMS RT=4.63 min, MH⁺ 345.3; ¹H NMR (DMSO): 8.65 (1H, br s), 8.08 (1H, d, J 8.3), 7.48 (1H, td, J 7.7 and 1.3), 7.30-7.18 (5H, br m), 7.15-6.99 (2H, br m), 3.67-3.58 (4H br m), 1.61-1.47 (2H, br m) and 1.39-1.25 (8H br m).

### N-(2-(4-Fluorophenyl)propan-2-yl)-2-(pyrrolidin-1-yl)quinazolin-4-amine (Compound 121)

LCMS RT=4.59 min, MH⁺ 351.3; ¹H NMR (DMSO): 8.21 (1H, d, J 8.3), 7.65 (1H, s), 7.50-7.35 (3H, br m), 7.21 (1H, d, J 7.2), 7.11-6.98 (3H, br m), 3.26-2.85 (4H, br m—obscured by water signal) and 1.83-1.68 (10H, br m).

### N-(2-(4-Fluorophenyl)propan-2-yl)-2-(piperidin-1-yl)quinazolin-4-amine (Compound 120)

LCMS RT=4.67 min, MH⁺ 365.2; ¹H NMR (CDCl₃): 7.59-7.34 (5H, br m), 7.07 (1H, td, J 7.5 and 1.3), 6.95 (2H, t, J 8.7), 5.77 (1H, br s), 3.54-3.43 (4H, br m), 1.83 (6H, s), 1.61-1.50 (2H, br m) and 1.41-1.29 (4H, br m).

### 1-(4-(Benzylamino)quinazolin-2-yl)piperidin-4-one (Compound 139)

LCMS RT=4.51 min, MH⁺ 333.3; ¹H NMR (DMSO): 8.71 (1H, br s), 8.08 (1H, d, J 8.1), 7.54 (1H, t, J 7.5), 7.42-7.25 (4H, br m), 7.25-7.16 (1H, br m), 7.13 (1H, t, J 7.3), 4.70 (2H, d, J 5.8), 4.07-3.97 (4H, br m) and 2.30-2.19 (4H, br m).

### N-(4-Chlorobenzyl)-4-(4,4-difluoropiperidin-1-yl)quinazolin-2-amine hydrochloride (Compound 146)

LCMS RT=4.54 min, MH⁺ 389.1; ¹H NMR (DMSO): 12.98 (1H, br s), 8.68 (1H, s), 7.97 (1H, d, J 8.4), 7.82 (1H, t, J 7.7), 7.62-7.52 (1H, br m), 7.46-7.35 (5H, br m), 4.76-4.59 (2H, br m), 4.08-3.96 (4H, br m) and 2.30-2.05 (4H, br m).

### 4-(4,4-Difluoropiperidin-1-yl)-N-(4-(trifluoromethyl)benzyl)quinazolin-2-amine (Compound 147)

LCMS RT=4.59 min, MH⁺ 423.2; ¹H NMR (DMSO): 7.76 (1H, d, J 8.1), 7.69-7.50 (6H, d, br m), 7.35 (1H, dd, J 8.5 and 1.0), 7.10 (1H, t, J 7.5), 4.62 (2H, d, J 6.0), 3.70-3.57 (4H, br m) and 2.30-1.91 (4H, br m).

### N-(2,4-Difluorobenzyl)-4-(4,4-difluoropiperidin-1-yl)quinazolin-2-amine (Compound 148)

LCMS RT=4.61 min, MH⁺ 391.3; ¹H NMR (DMSO): 7.76 (1H, d, J 8.4), 7.59-7.33 (4H, br m), 7.19 (1H, br m), 7.11 (1H, t, J 7.7), 7.01 (1H, br m), 4.55 (2H, d, J 5.8), 3.70-3.61 (4H, br m) and 2.25-2.00 (4H, br m).

### 4-(4,4-Difluoropiperidin-1-yl)-N-(4-fluorobenzyl)quinazolin-2-amine hydrochloride (Compound 149)

LCMS RT=4.49 min, MH⁺ 373.1; ¹H NMR (DMSO): 12.74 (1H, br s), 8.59 (1H, br s), 7.96 (1H, d, J 8.2), 7.86-7.75 (1H, br m), 7.64-7.50 (1H, br m), 7.49-7.33 (3H, br m), 7.18 (2H, d, J 8.9), 4.70-4.59 (2H, br m), 4.12-3.92 (4H, br m) and 2.29-2.06 (4H, br m).

### 2-(4,4-Difluoropiperidin-1-yl)-N-(4-(trifluoromethoxy)benzyl)quinazolin-4-amine (Compound 163)

LCMS RT=4.21 min, MH⁺ 439.1; ¹H NMR (DMSO): 8.75 (1H, br s), 8.06 (1H, d, J 8.4), 7.57-7.45 (3H, br s), 7.33-7.27 (3H, br s), 7.13 (1H, td, J 7.6 and 1.1), 4.68 (2H, d, J 5.8), 3.86-3.79 (4H, br m) and 1.86-1.69 (4H, br s).

### N²,N⁴-bis(4-Fluorobenzyl)thieno[3,2-d]pyrimidine-2,4-diamine (Compound 166)

LCMS RT=1.65 min, MH⁺ 383; ¹H NMR (CDCl₃): 7.49 (1H, d, J 5.3), 7.25-7.16 (4H, br m), 7.05 (1H, d, J 5.3), 6.96-6.85 (4H, br m), 5.32 (1H, br s), 5.04 (1H, br s), 4.64 (2H, d, J 5.8) and 4.54 (2H, d, J 6.0).

### N-((5-Methylfuran-2-yl)methyl)-2-(piperidin-1-yl)quinazolin-4-amine (Compound 167)

LCMS RT=1.61 min; MH⁺ 323.2; ¹H NMR (DMSO): 8.36 (1H, t, J 5.7), 7.80 (1H, dd, J 8.3 and 1.0), 7.47 (1H, ddd, J 8.4, 6.9 and 1.4), 7.24 (1H, dd, J 8.5 and 0.8), 7.01 (1H, ddd, J 8.1, 6.9 and 1.2), 6.14 (1H, d, J 3.0), 5.99-5.96 (1H, m), 4.59 (2H, d, J 5.6), 3.82-3.76 (4H, m), 2.22 (3H, s), 1.67-1.56 (2H, m) and 1.55-1.45 (4H, m).

### tert-Butyl 4-(4-fluorobenzylamino)-2-(piperidin-1-yl)-5,6-dihydropyrido[3,4-d]pyrimidine-7(8H)-carboxylate (Compound 184)

LCMS RT=1.49 min; MH⁺ 442.1; ¹H NMR (DMSO): 7.37-7.30 (2H, m), 7.16 (1H, br s), 7.13-7.05 (2H, m), 4.50-4.45 (2H, m), 4.10 (2H, br s), 3.59-3.51 (4H, m), 2.35-2.29 (2H, m), 1.58-1.49 (2H, m) and 1.44-1.32 (13H, tBu s and m).

### Method 2b

### Compounds of General Formula (I)

### N-Benzyl-2-(pyrrolidin-1-yl)quinazolin-4-amine (Compound 22)

N-Benzyl-2-chloroquinazolin-4-amine (108 mg, 0.4 mmol) was dissolved in IMS (Solvent S, 3 mL). NEt₃ (112 □L, 0.8 mmol, 2 eq. EB) was added, followed by pyrrolidine (100 □L, 1.2 mmol, 3 eq. E) was added and the mixture was heated to 150° C. (Temperature K) for 10 min (Time T) under microwave irradiation. The mixture was concentrated in vacuo, diluted with EtOAc and extracted with EtOAc (2×20 mL) from aqueous K₂CO₃ or NaHCO₃. The organic layer was washed with brine, dried (MgSO₄), filtered and concentrated in vacuo. The crude product was purified by column chromatography (1:1 petrol-EtOAc) to afford the title compound as a white solid (105 mg, 85%).

LCMS RT=4.57 min, MH⁺ 305.2; ¹H NMR (DMSO): 8.52-8.45 (1H, m), 7.97 (1H, br d), 7.47 (1H, ddd, J 8.2, 6.9 and 1.2), 7.40-7.34 (2H, m), 7.33-7.24 (3H, m), 7.23-7.16 (1H, m), 7.01 (1H, br t), 4.68 (2H, s), 3.49-3.40 (4H, m) and 1.88-1.82 (4H, m).

The following compounds were prepared in a similar manner, purifying by crystallisation or column chromatography where necessary:

### N-Benzyl-4-(piperidin-1-yl)quinazolin-2-amine (Compound 24)

LCMS RT=4.89 min, MH⁺ 319.2; ¹H NMR (CDCl₃): 7.71 (1H, br d), 7.54-7.49 (2H, m), 7.44-7.39 (2H, m), 7.37-7.23 (4H, m), 7.11-7.06 (1H, m), 5.26 (1H, br s), 4.74 (2H, d, J 5.8), 3.64-3.56 (4H, m) and 1.83-1.69 (6H, m)

### N⁴-Benzyl-N²,N²-diethylquinazoline-2,4-diamine (Compound 25)

LCMS RT=8.55 min, MH⁺ 307.2; ¹H NMR (DMSO): 8.52 (1H, t, J 5.8), 8.03 (1H, d, J 8.2), 7.49 (1H, ddd, J 8.4, 7.0 and 1.4), 7.40-7.20 (6H, m), 7.04 (1H, ddd, J 8.1, 7.0 and 1.1), 4.70 (2H, d, J 5.7), 3.56 (4H, q, J 6.9) and 1.05 (3H, t, J 6.7).

### N-Benzyl-2-(4-(ethylsulfonyl)piperazin-1-yl)quinazolin-4-amine (Compound 26)

LCMS RT=6.26 min, MH⁺ 412.1; ¹H NMR (DMSO): 8.66 (1H, t, J 6.0), 8.06 (1H, d, J 8.4), 7.53 (1H, ddd, J 8.2, 6.9 and 1.2), 7.41-7.20 (6H, m), 7.11 (1H, ddd, J 8.1, 7.1 and 1.1), 4.69 (2H, d, J 5.7), 3.81 (4H, t, J 4.7), 3.14 (4H, t, J 4.7), 2.84 (3H, s), 3.02 (2H, q, J 7.4) and 1.20 (3H, t, J 7.4).

### Ethyl 2-(4-(4-(benzylamino)quinazolin-2-yl)piperazin-1-yl)acetate (Compound 27)

LCMS RT=6.44 min, MH⁺ 406.2; ¹H NMR (DMSO): 8.58 (1H, t, J 6.0), 8.03 (1H, d, J 8.2), 7.50 (1H, ddd, J 8.4, 7.2 and 1.2), 7.39-7.18 (6H, m), 7.07 (1H, ddd, J 8.1, 7.1 and 1.1), 4.68 (2H, d, J 5.4), 4.08 (2H, q, J 7.1), 3.72 (4H, t, J 4.0), 3.23 (2H, s) and 1.18 (t, J 7.1).

### N⁴-Benzyl-N²-(4-methylbenzyl)quinazoline-2,4-diamine (Compound 28)

LCMS RT=7.63 min, MH⁺ 355.2; ¹H NMR (DMSO): 8.41 (1H, br s), 7.99 (1H, d, J 7.5), 7.47 (1H, ddd, J 8.3, 7.1 and 1.1), 7.34-7.14 (8H, m), 7.05-7.00 (4H, m), 4.70 (2H, d, J 5.4), 4.44 (2H, d, J 6.2) and 2.24 (3H, s).

### N²,N⁴-Dibenzyl-N²-methylquinazoline-2,4-diamine (Compound 29)

LCMS RT=8.18 min, MH⁺ 355.2; ¹H NMR (DMSO): 8.59 (1H, br s), 8.05 (1H, d, J 7.4), 7.51 (1H, ddd, J 8.2, 6.8 and 1.1), 7.32-7.16 (11H, m), 7.07 (1H, ddd, J 8.2, 7.1 and 1.0), 4.84 (2H, s), 4.68 (2H, d, J 5.9) and 3.04 (3H, s).

### N-Benzyl-2-(3,4-dihydroisoquinolin-2(1H)-yl)quinazolin-4-amine (Compound 30)

LCMS RT=8.54 min, MH⁺ 367.2; ¹H NMR (DMSO): 8.63 (1H, t, J 5.4), 8.03 (1H, d, J 7.3), 7.51 (1H, ddd, J 8.4, 6.8 and 1.4), 7.43-7.41 (2H, m), 7.34-7.29 (3H, m), 7.24-7.11 (5H, m), 7.08 (1H, ddd, J 8.2, 7.1 and 1.2), 4.86 (2H, s), 4.73 (2H, d, J 5.8), 3.97 (2H, t, J 5.8) and 2.76 (2H, t, J 5.5).

### tert-Butyl 4-(4-(benzylamino)quinazolin-2-yl)piperazine-1-carboxylate (Compound 31)

LCMS RT=7.87 min, MH⁺ 420.2; ¹H NMR (CDCl3): 7.58-7.48 (3H, m), 7.43-7.32 (5H, m), 7.09 (1H, m), 5.80 (1H, br s), 4.82 (2H, d, J 5.6), 3.89 (4H, t, J 4.9), 3.50 (4H, t, J 5.2) and 1.51 (9H, s).

### N⁴-Benzylquinazoline-2,4-diamine (Comparative Compound 3)

LCMS RT=5.65 min, MH⁺ 251.1; ¹H NMR (DMSO): 8.37 (1H, t, J 5.8, NH), 8.01 (1H, dd, J 8.4 and 1.1), 7.48 (1H, ddd, J 8.3, 7.0 and 1.3), 7.39-7.27 (4H, m), 7.26-7.18 (2H, m), 7.03 (1H, ddd, J 8.0, 6.9 and 1.2), 6.03 (2H, s, NH) and 4.73 (2H, d, J 5.9).

### N⁴-(4-Chlorobenzyl)-N²-(4-methylbenzyl)quinazoline-2,4-diamine (Compound 76)

LCMS RT=4.69 min, MH⁺ 389.2; ¹H NMR (DMSO): 8.49-8.40 (1H, br s), 7.99-7.96 (1H, m), 7.47 (1H, ddd, J 8.4, 6.9 and 1.4), 7.37-7.27 (4H, m), 7.23-6.96 (7H, m), 4.69-4.64 (2H, m), 4.46-4.40 (2H, m) and 2.24 (3H, s).

### N⁴-(4-Trifluoromethylbenzyl)-N²-(4-methylbenzyl)quinazoline-2,4-diamine (Compound 79)

LCMS RT=4.74 min, MH⁺ 423.3; ¹H NMR (DMSO): 8.53 (1H, br s), 7.99 (1H, dd, J 8.3 and 0.9), 7.66-7.43 (5H, m), 7.22 (1H, d, J 8.4), 7.17-6.93 (6H, m), 4.79-4.73 (2H, m), 4.45-4.38 (2H, m) and 2.22 (3H, s).

### Method 2c

### Compounds of General Formula (I)

### 4-(Piperidin-1-yl)-N-(4-(trifluoromethyl)benzyl)quinazolin-2-amine hydrochloride (Compound 109)

2-Chloro-4-(piperidin-1-yl)quinazoline (100 mg, 0.40 mmol) was dissolved in MeCN (Solvent S, 2 mL). 4-(Trifluoromethyl)benzylamine (60.5 □L, 0.42 mmol, 1.05 eq. E) was added and the mixture was heated to 180° C. (Temperature K) for 3×10 min (Time T) under microwave irradiation. The mixture was concentrated and the residual solid suspended in EtOAc. The mixture was filtered, and the collected solid washed with EtOAc, then dried under vacuum to give the title compound as a yellow solid (92 mg, 54%).

LCMS RT=4.65, MH⁺ 387.2 ¹H NMR (DMSO): 12.91 (1H, br s), 8.62 (1H, s), 7.91 (1H, d, J 8.3), 7.79 (1H, t, J 7.8), 7.72 (2H, d, J 8.1), 7.64-7.47 (3H, br m), 7.37 (1H, t, J 7.8), 4.73 (2H, br s), 3.90 (4H, br s) and 1.63 (6H, br s).

The following compounds were prepared in a similar manner, purifying by crystallisation or column chromatography where necessary:

### N²,N⁴-bis(4-Fluorobenzyl)quinazoline-2,4-diamine hydrochloride (Compound 119)

LCMS RT=12.92 min, MH⁺ 377.2; ¹H NMR (DMSO): 12.60 (1H, br s), 10.18 (1H, s), 8.49 (1H, s), 8.31 (1H, d, J 8.3), 7.85-7.78 (1H, m), 7.55-7.39 (2H, m), 7.36-7.23 (4H, br m.), 7.13-7.00 (4H, br m), 4.75 (2H, d, J 8.3) and 4.68-4.59 (2H, br m).

### N⁴-(4-Fluorobenzyl)-N²-(4-fluorophenyl)quinazoline-2,4-diamine hydrochloride (Compound 118)

LCMS RT=4.62 min, MH⁺ 363.2; ¹H NMR (DMSO): 12.64 (1H, br s), 10.38 (1H, s), 8.42 (1H, d, J 8.0), 7.90-7.80 (1H, m), 7.63-7.56 (1H, m), 7.53-7.43 (3H, m), 7.40-7.31 (2H, m), 7.29-7.11 (4H, m) and 4.72 (2H, d, J 5.3).

### N-(2,4-Dichlorobenzyl)-4-(piperidin-1-yl)quinazolin-2-amine hydrochloride (Compound 112)

LCMS RT=4.70 min, MH⁺ 387.1; ¹H NMR (DMSO): 12.85 (1H, br s), 8.55 (1H, s), 7.92 (1H, d, J 8.3), 7.79 (1H, t, J 7.7), 7.66 (1H, d, J 1.7), 7.60-7.50 (1H, br m), 7.49-7.32 (3H, br m), 4.70 (2H, d, J 5.7), 3.98-3.84 (4H, br m) and 1.75-1.55 (6H, br m).

### N-(4-Fluorobenzyl)-4-(piperidin-1-yl)quinazolin-2-amine hydrochloride (Compound 111)

LCMS RT=4.63, MH⁺ 337.3 ¹H NMR (DMSO): 12.63 (1H, br s), 7.91 (1H, d, J 8.2), 7.78 (1H, d, J 7.5), 7.60-7.31 (3H, br m), 7.23-7.12 (2H, t, J 8.9), 4.63 (2H, br s), 4.01-3.86 (4H, br m) and 1.77-1.58 (6H, br m).

### N-(4-Chlorobenzyl)-4-(piperidin-1-yl)quinazolin-2-amine hydrochloride (Compound 110)

LCMS RT=4.67, MH⁺ 353.2 ¹H NMR (DMSO): 12.55 (1H, br s), 8.49 (1H, s), 7.91 (1H, d, J 8.0), 7.78 (1H, t, J 7.8), 7.60-7.48 (1H, br s), 7.45-7.32 (5H, br s), 4.63 (2H, d, J 5.5), 3.97-3.87 (4H, br m) and 1.77-1.57 (6H, br m)

### N-Benzyl-2-(4-(4-methoxyphenyl)piperazin-1-yl)quinazolin-4-amine hydrochloride (Compound 136)

LCMS RT=4.65 min, MH⁺ 426.3; ¹H NMR (DMSO): 12.08 (1H, br s), 10.19 (1H, br s), 8.35 (1H, d, J 8.4), 7.87-7.75 (2H, br m), 7.51-7.40 (3H, br m), 7.36 (2H, t, J 7.2), 7.33-7.24 (1H, br m), 6.96 (2H, d, J 9.1), 6.85 (2H, d, J 9.0), 4.82 (2H, d, J 5.3), 4.05-3.95 (4H, br m), 3.69 (3H, s) and 3.17-3.07 (4H, br m).

### N-Benzyl-2-(4-(4-(trifluoromethyl)phenyl)piperazin-1-yl)quinazolin-4-amine hydrochloride Compound 135)

LCMS RT=4.77 min, MH⁺ 464.3; ¹H NMR (DMSO): 12.09 (1H, br s), 10.20 (1H, br s), 8.35 (1H, d, J 8.4), 7.88-7.75 (2H, br m), 7.55 (2H, d, J 8.6), 7.51-7.41 (3H, br m), 7.37 (2H, t, J 7.3), 7.32-7.25 (1H, br m), 7.11 (2H, d, J 8.7), 4.83 (2H, d, J 5.4), 4.07-3.97 (4H, br m) and 3.53-3.41 (4H, br m).

### N-Benzyl-2-(4-(4-chlorophenyl)piperazin-1-yl)quinazolin-4-amine hydrochloride (Compound 134)

LCMS RT=4.73 min, MH⁺ 430.3; ¹H NMR (DMSO): 12.17 (1H, br s), 10.24 (1H, br s), 8.37 (1H, d, J 8.2), 7.50-7.40 (4H, br m), 7.35 (2H, t, J 7.4), 7.32-7.24 (4H, br m), 7.00 (2H, d, J 9.0), 4.82 (2H, d, J 5.5), 4.06-3.97 (4H, br m) and 3.32-3.23 (4H, br m—obscured by water signal).

### N-Benzyl-2-(4-phenylpiperazin-1-yl)quinazolin-4-amine hydrochloride (Compound 133)

LCMS RT=4.67 min, MH⁺ 396.2; ¹H NMR (DMSO): 12.14 (1H, br s), 10.22 (1H, br s), 8.36 (1H, d, J 8.0), 7.86-7.78 (2H, br m), 7.51-7.41 (3H, br m), 7.36 (2H, t, J 7.3), 7.32-7.21 (3H, br m), 6.99 (2H, d, J 8.0), 6.83 (1H, t, J 7.3), 4.83 (2H, d, J 5.7), 4.06-3.97 (4H, br m) and 3.32-3.20 (4H, br m—obscured by water signal).

### N-Benzyl-2-(4-phenylpiperidin-1-yl)quinazolin-4-amine hydrochloride (Compound 132)

LCMS RT=4.70 min, MH⁺ 395.2; ¹H NMR (DMSO): 11.99 (1H, br s), 10.18 (1H, br s), 8.36 (1H, d, J 8.2), 7.82 (2H, d, J 3.9), 7.50-7.39 (3H, br m), 7.37-7.16 (8H, br m), 4.84-4.68 (4H, br m), 3.29-3.13 (2H, br m—obscured by NMR solvent signal), 2.97-2.83 (1H, br), 1.94-1.83 (2H, br m) and 1.74-1.54 (2H, br m).

### N⁴-Benzyl-N²-(4-methoxybenzyl)quinazoline-2,4-diamine hydrochloride (Compound 129)

LCMS RT=4.63 min, MH⁺ 371.2; ¹H NMR (DMSO):): 12.53 (1H, br s), 10.18 (1H, br s), 8.45 (1H, br s), 8.32 (1H, t, J 8.2), 7.80 (1H, t, J 7.7), 7.53-7.12 (9H, br m), 7.00-6.73 (2H, br m), 4.81 (2H, d, J 5.8), 4.61-4.52 (2H, br m) and 3.71 (3H, s).

### N⁴-Benzyl-N²-(4-(trifluoromethyl)benzyl)quinazoline-2,4-diamine hydrochloride (Compound 128)

LCMS RT=4.69 min, MH⁺ 409.2; ¹H NMR (DMSO): 12.72 (1H, br s), 10.17 (1H, br s), 8.57 (1H, br s), 8.32 (1H, d, J 8.2), 7.82 (1H, t, J 7.6), 7.69-7.38 (6H, br m), 7.31-7.11 (5H, br m) and 4.81-4.67 (4H, br m).

### N⁴-Benzyl-N²-(4-chlorobenzyl)quinazoline-2,4-diamine hydrochloride (Compound 127)

LCMS RT=4.66 MH⁺ 375.2; NMR (DMSO): 12.67 (1H, br s), 10.19 (1H, br s), 8.52 (1H, br s), 8.32 (1H, d, J 8.2), 7.81 (1H, t, J 7.5), 7.58-7.16 (11H, br m), 4.81-4.70 (2H, br m) and 4.69-4.58 (2H, br m).

### N⁴-Benzyl-N²-(4-fluorobenzyl)quinazoline-2,4-diamine hydrochloride (Compound 126)

LCMS RT=4.65 min, MH⁺ 359.2; ¹H NMR (DMSO): 12.58 (1H, br s), 10.18 (1H, br s), 8.49 (1H, br s), 8.32 (1H, d, J 8.2), 7.81 (1H, t, J 7.6), 7.56-6.98 (9H, br m), 7.16-6.98 (2H, br m), 4.78 (2H, t, J 5.7) and 4.68-4.57 (2H, br m).

### N-Benzyl-2-thiomorpholinoquinazolin-4-amine hydrochloride (Compound 140)

LCMS RT=4.64 min, MH⁺ 337.2; ¹H NMR (DMSO): 12.04 (1H, s), 10.24 (1H, br s), 8.36 (1H, d, J 8.1), 7.82 (2H, d, J 3.6), 7.54-7.20 (6H, br m), 4.77 (2H; d, J 5.8), 4.18-4.08 (4H, br m) and 2.75-2.61 (4H, br m).

### N-(1-(4-Chlorophenyl)cyclopropyl)-2-(piperidin-1-yl)quinazolin-4-amine hydrochloride (Compound 141)

LCMS RT=4.70 min, MH⁺ 379.2; ¹H NMR (DMSO): 12.02 (1H, br s), 10.16 (1H, br s), 8.40 (1H, d, J 8.2), 7.81 (2H, d, J 4.1), 7.49-7.41 (1H, br m), 7.37-7.25 (4H, br m), 3.79-3.67 (4H, br m), 1.68-1.56 (2H, br m) and 1.54-1.37 (8H, br m).

### N-(1-(4-Chlorophenyl)cyclopropyl)-2-(pyrrolidin-1-yl)quinazolin-4-amine hydrochloride (Compound 142)

LCMS RT=4.68 min, MH⁺ 365.2; ¹H NMR (DMSO): 11.89 (1H, br s), 10.16 (1H, br s), 8.40 (1H, d, J 8.2), 7.86-7.74 (2H, br m), 7.49-7.39 (1H, m), 7.39-7.28 (4H, br m), 3.68-3.38 (4H, br m—obscured by water signal), 2.12-1.78 (4H, br m) and 1.52-1.33 (4H, br m).

### N-(4-Fluorobenzyl)-6,7-dimethoxy-2-(piperidin-1-yl)quinazolin-4-amine hydrochloride (Compound 150)

LCMS RT=4.66 min, MH⁺ 397.2; ¹H NMR (DMSO): 11.73 (1H, br s), 9.84 (1H, br s), 7.81 (1H, s), 7.49-7.40 (2H, br m), 7.32 (1H, s), 7.18 (2H, t, J 8.9), 4.75 (2H, d, J 5.6), 3.89 (3H, s), 3.86 (3H, s), 3.81-3.73 (4H, br m) and 1.70-1.49 (6H, br m).

### 2-(4,4-Difluoropiperidin-1-yl)-N-(4-fluorobenzyl)-6,7-dimethoxyquinazolin-4-amine hydrochloride (Compound 151)

LCMS RT=4.63 min, MH⁺ 433.3; ¹H NMR (DMSO): 12.13 (1H, br s), 9.98 (1H, br s), 7.85 (1H, s), 7.49-7.42 (2H, br m), 7.34 (1H, s), 7.18 (2H, t, J 8.8), 4.77 (2H, d, J 5.4), 3.98-3.90 (4H, br m—overlaps adjacent methyl signal), 3.90 (3H, s—overlaps adjacent signal), 3.87 (3H, s) and 2.17-1.99 (4H, br m).

### N²-(4-Fluorobenzyl)-N⁴-((5-methylfuran-2-yl)methyl)quinazoline-2,4-diamine hydrochloride (Compound 158)

LCMS RT=4.35 min, MH⁺ 363.2; ¹H NMR (DMSO): 12.66 (1H, br m), 10.14 (1H, br s), 8.56 (1H, br s), 8.28 (1H, d, J 8.5), 7.83 (1H, t, J 7.9), 7.57-7.28 (4H, br m), 7.22-7.05 (3H, br m), 6.46 (1H, br s), 4.86-4.79 (2H, br m), 4.73-4.62 (2H, br s), methylsignal completely obscured by NMRsolvent signal (3H).

### N²-(4-Fluorobenzyl)-N4-((5-(trifluoromethyl)furan-2-yl)methyl)quinazoline-2,4-diamine hydrochloride (Compound 159)

LCMS RT=4.58 min, MH⁺ 417.2; NMR (DMSO): 12.63 (1H, br m), 10.12 (1H, br s), 8.55 (1H, br s), 8.28 (1H, d, J 8.1), 7.83 (1H, t, J 7.8), 7.57-7.29 (4H, br m), 7.22-7.03 (3H, br m), 6.46 (1H, br s), 4.84-4.79 (2H, br m) and 4.72-4.63 (2H, br s).

### 2-(Piperidin-1-yl)-N-((5-(trifluoromethyl)furan-2-yl)methyl)quinazolin-4-amine hydrochloride (Compound 160)

LCMS RT=4.53 min, MH⁺ 377.1; ¹H NMR (DMSO): 11.81 (1H, br s), 10.02 (1H, br s), 8.25 (1H, d, J 8.2), 7.87-7.68 (2H, br m), 7.45 (1H, t, J 7.4), 7.23-7.17 (1H, br m), 6.63 (1H, d, J 3.6), 4.84 (2H, d, J 5.3), 3.90-3.79 (4H, br m) and 1.72-1.54 (6H, br m).

### 2-(4,4-Difluoropiperidin-1-yl)-N-((5-(trifluoromethyl)furan-2-yl)methyl)quinazolin-4-amine hydrochloride (Compound 161)

LCMS RT=4.53 min, MH⁺ 413.1; ¹H NMR (DMSO): 12.13 (1H, br s), 10.14 (1H, br s), 8.32-8.24 (1H, br s), 7.92-7.65 (2H, br m), 7.53-7.41 (1H, br m), 7.24-7.19 (1H, br m), 6.71-6.64 (1H, br s), 4.91-4.88 (2H, br s), 4.05-3.94 (4H, br s) and 2.23-2.03 (4H, br s).

### N²,N⁴-bis(4-Fluorobenzyl)-6,7-dimethoxyquinazoline-2,4-diamine (Compound 168)

LCMS RT=4.64 min, MH⁺ 377.2; ¹H NMR (DMSO): 12.27 (1H, br s), 9.77 (1H, br s), 8.38 (1H, s), 7.70 (1H, s), 7.35-7.21 (4H, br m), 7.14-6.93 (5H, br m), 4.72 (2H, s), 4.57 (2H, s), 3.89 (3H, s) and 3.83 (3H, s).

### N-(4-Fluorobenzyl)-2-(pyrrolidin-1-yl)pyrido[2,3-d]pyrimidin-4-amine (Compound 173)

LCMS RT=1.35 min, MH⁺ 324.2; ¹H NMR (DMSO): 8.73 (1H, br s), 8.61 (1H, dd, J 4.5 and 1.9), 8.38 (1H, dd, J 8.0 and 1.9), 7.46-7.39 (2H, br m), 7.14 (2H, t, J 9.0), 7.01 (1H, dd, J 8.1 and 4.4), 4.67 (2H, d, J 5.6), 3.53-3.46 (4H, br m) and 1.97-1.84 (4H, br m).

### N-(4-Fluorobenzyl)-2-(piperidin-1-yl)pyrido[2,3-d]pyrimidin-4-amine (Compound 174)

LCMS RT=1.37 min, MH⁺ 338.2; ¹H NMR (DMSO): 8.76 (1H, br s), 8.61 (1H, dd, J 4.4 and 1.9), 8.38 (1H, dd, J 8.0 and 1.9), 7.44-7.36 (2H, br m), 7.13 (2H, t, J 8.8), 7.03 (1H, dd, J 8.1 and 4.5), 4.65 (2H, d, J 5.7), 3.79-3.72 (4H, br m), 1.65-1.54 (2H, br m) and 1.49-1.38 (4H, br m).

### N-(4-Fluorobenzyl)-2-(isoindolin-2-yl)quinazolin-4-amine hydrochloride (Compound 179)

LCMS RT=1.49 min, MH⁺ 371.1; ¹H NMR (DMSO): 12.08 (1H, br s), 10.23 (1H, br s), 8.39 (1H, d, J 8.2), 7.91-7.83 (2H, br m), 7.61-7.37 (7H, br m), 7.22 (2H, t, J 9.0), 5.04 (4H, d, J 5.2) and 4.89 (2H, d, J 5.7).

### 4-(3,4-Dihydroisoquinolin-2(1H)-yl)-2-(pyrrolidin-1-yl)quinazoline (Compound 186)

LCMS RT=1.66 min, MH⁺ 331.2; ¹H NMR (DMSO-d6): 12.10 (1H, br s), 8.17 (1H, d, J 8.3), 7.93 (1H, d, J 8.0), 7.84 (1H, t, J 7.6), 7.44 (1H, t, J 7.6), 7.38-7.22 (4H, m), 5.14 (2H, s), 4.18 (2H, br t, J 5.8), 3.72 (4H, br s), 3.11 (2H, br t, J 5.6) and 2.16-1.90 (4H, br m).

### 2-(Pyrrolidin-1-yl)-N-(thiophen-2-ylmethyl)quinazolin-4-amine (Compound 187)

LCMS RT=1.63 min, MH⁺ 311.2; ¹H NMR (DMSO-d6): 11.88 (1H, br s), 10.15 (1H, br s), 8.29 (1H, d, J 8.0), 7.86-7.78 (2H, m), 7.48-7.40 (2H, m), 7.18 (1H, dd, J 3.5 and 1.3), 7.01 (1H, dd, J 5.1 and 3.5), 4.97 (2H, d, J 5.8), 3.85-3.62 (4H, m) and 2.16-1.92 (4H, m).

### 2-(Azepan-1-yl)-N-benzylquinazolin-4-amine (Compound 197)

LCMS RT=1.66 min; MH⁺ 333.3; ¹H NMR (DMSO): 11.72 (1H, s), 10.14 (1H, br s), 8.36 (1H, d, J 7.9), 7.91-7.77 (2H, m), 7.49-7.21 (6H, m), 4.77 (2H, d, J 5.5), 3.81-3.71 (4H, m) and 1.87-1.38 (8H, m).

### 2-(7-Bromo-3,4-dihydroisoquinolin-2(1H)-yl)-N-(4-fluorobenzyl)quinazolin-4-amine (Compound 193)

LCMS RT=1.70 min, MH⁺ 464.9; ¹H NMR (DMSO-d6): 12.26 (1H, s), 10.30 (1H, s), 8.40 (1H, d, J 8.1), 7.93 (1H, d, J 8.3), 7.84 (1H, t, J 7.7), 7.60-7.42 (5H, m), 7.26-7.14 (3H, m), 5.04 (2H, s), 4.85 (2H, d, J 5.5), 4.05 (2H, t, J 6.1) and 3.00-2.90 (2H, m).

### Cyclopropyl(4-(4-(4-fluorobenzylamino)quinazolin-2-yl)piperazin-1-yl)methanone hydrochloride (Compound 202)

LCMS RT=1.55 min; MH⁺ 406.1; ¹H NMR (DMSO): 12.24 (1H, s), 10.30 (1H, s), 8.43 (1H, d, J 8.2), 7.90-7.78 (2H, m), 7.53-7.42 (3H, m), 7.23-7.13 (2H, m), 4.78 (2H, d, J 5.5), 4.03-3.77 (6H, m), 3.61 (2H, br s), 2.07-1.98 (1H, m) and 0.80-0.71 (4H, m).

### (4-(4-(4-Fluorobenzylamino)quinazolin-2-yl)piperazin-1-yl)(pyrrolidin-1-yl)methanone hydrochloride (Compound 205)

LCMS RT=1.61 min; MH⁺ 435.1; ¹H NMR (DMSO): 12.16 (1H, s), 10.25 (1H, br s), 8.37 (1H, d, J 8.2), 7.87-7.78 (2H, m), 7.52-7.41 (3H, m), 7.22-7.13 (2H, m), 4.77 (2H, d, J 5.7), 3.93-3.84 (4H, m), 3.34-3.22 (8H, m, obsc. by water signal) and 1.84-1.69 (4H, m).

### Method 2d

### Compounds of General Formula (I)

### N-(4-Chlorobenzyl)-2-(3,4-dihydroisoquinolin-2(1H)-yl)quinazolin-4-amine hydrochloride (Compound 106)

N-(4-Chlorobenzyl)-2-chloroquinazolin-4-amine (75 mg, 0.25 mmol) was dissolved in MeCN (Solvent S, 3 mL). NEt₃ (70 □L, 0.5 mmol, 2 eq. EB) was added, followed by 1,2,3,4-tetrahydroisoquinoline (33 □L, 0.26 mmol, 1.05 eq. E) was added and the mixture was heated to 180° C. (Temperature K) for 15 min (Time T) under microwave irradiation. The mixture was concentrated in vacuo, diluted with EtOAc and extracted with EtOAc (2×20 mL) from aqueous K₂CO₃ or NaHCO₃. The organic layer was washed with brine, dried (MgSO₄), filtered and concentrated in vacuo. The crude product was first purified by column chromatography (100% petrol-100% EtOAc), then dissolved in THF and 4M HCl in dioxane (0.75 eq.) added. The mixture was concentrated under reduced pressure to give a solid which was washed with EtOAc under suction and dried under vacuum to afford the title compound as a white solid (11.9 mg, 11%).

LCMS RT=4.72 min, MH⁺ 401.2 ¹H NMR (DMSO): 11.96 (1H, br s), 10.11 (1H, br s), 8.31 (1H, d, J 8.9), 7.88-7.71 (2H, br m), 7.53-7.34 (6H, br m), 7.29-7.18 (4H, br s), 4.91 (2H, s), 4.82 (2H, d, J 5.5), 3.98 (2H, m) and 2.94 (2H, br s).

The following compounds were prepared in a similar manner, purifying by crystallisation or column chromatography where necessary:

### 2-(3,4-Dihydroisoquinolin-2(1H)-yl)-N-(4-(trifluoromethyl)benzyl)quinazolin-4-amine hydrochloride (Compound 114)

LCMS RT=4.74 min, MH⁺ 435.3 ¹H NMR (DMSO): 11.94 (1H, br s), 10.22 (1H, br s), 8.33 (1H, d, J 8.8), 7.90-7.63 (6H, br m), 7.48 (1H, br s), 7.28-7.16 (4H, br m), 4.96-4.84 (4H, br m), 3.95 (2H, t, J 6.2) and 2.90 (2H, br s).

### 2-(3,4-Dihydroisoquinolin-2(1H)-yl)-4-(piperidin-1-yl)quinazoline hydrochloride (Compound 113)

LCMS RT=4.67 min, MH⁺ 345.3; ¹H NMR (DMSO): 12.02 (1H, br s), 7.94 (1H, d, J 8.2), 7.87-7.77 (2H, br m), 7.45-7.36 (1H, br m), 7.32-7.24 (4H, br m), 4.97 (2H, s), 4.06-3.92 (6H, br m), 3.00 (2H, t, J 5.4) and 1.79-1.71 (6H, br m).

### Method 2e

### Compounds of General Formula (I)

### N-Benzyl-2-(6,7-dimethoxy-3,4-dihydroisoquinolin-2(1H)-yl)quinazolin-4-amine (Compound 181)

N-Benzyl-2-chloroquinazolin-4-amine (81 mg, 0.30 mmol) was suspended in MeCN (Solvent S, 2 mL), and treated with potassium carbonate (83 mg, 0.60 mmol, 2 eq. EB) and 6,7-dimethoxy-1,2,3,4-tetrahydroisoquinoline hydrochloride (69 mg, 0.30 mmol, 1 eq. E). The mixture was heated to 160° C. (Temperature K) for 10 minute (Time T) under microwave irradiation. After cooling to room temperature, the crude reaction mixture was poured into aq. NaHCO₃ solution and extracted with EtOAc. The organic layer was dried (MgSO₄), filtered and absorbed onto silica. The crude product was purified by column chromatography (1:1 petrol-EtOAc) to afford the title compound as a yellow solid (79 mg, 77%).

LCMS RT=1.61 min, MH⁺ 427.4; ¹H NMR (DMSO-d₆): 8.62 (1H, br s), 8.04 (1H, d, J 8.1), 7.51 (1H, td, J 7.6 and 1.3), 7.42 (2H, d, J 8.3), 7.35-7.27 (3H, br m), 7.24-7.19 (1H, br m), 7.07 (1H, td, J 7.5 and 1.1), 6.78 (1H, s), 6.69 (1H, s), 4.78 (2H, s), 4.74 (2H, d, J 5.8), 3.95 (2H, t, J 5.9), 3.74 (3H, s), 3.70 (3H, s) and 2.74-2.62 (2H, br m).

The following compounds were prepared in a similar manner, purifying by crystallization or column chromatography where necessary:

### 4-(6,7-Dimethoxy-3,4-dihydroisoquinolin-2(1H)-yl)-2-(pyrrolidin-1-yl)quinazoline (Compound 188)

LCMS RT=1.62 min, MH⁺ 391.2; ¹H NMR (DMSO-d6): 7.83 (1H, d, J 8.1), 7.54 (1H, t, J 7.8), 7.39 (1H, d, J 8.4), 7.10 (1H, t, J 7.6), 6.81 (2H, d, J 9.4), 4.76 (2H, s), 3.88 (2H, br t, J 5.8), 3.75 (3H, s), 3.73 (3H, s), 3.64-3.54 (4H, m), 3.03 (2H, br t, J 5.6) and 1.98-1.90 (4H, m).

### 4-(6,7-Dimethoxy-3,4-dihydroisoquinolin-2(1H)-yl)-2-(piperidin-1-yl)quinazoline (Compound 192)

LCMS RT=1.64 min, MH⁺ 405.2; ¹H NMR (DMSO-d6): 7.83 (1H, d, J 8.3), 7.55 (1H, t, J 7.7), 7.37 (1H, d, J 8.5), 7.12 (1H, t, J 7.5), 6.82 (2H, d, J 15.2), 4.74 (2H, s), 3.90-3.79 (6H, m), 3.75 (3H, s), 3.73 (3H, s), 3.02 (2H, br t, J 5.7) and 1.70-1.50 (6H, m).

### Method 2f

### Compounds of General Formula (I)

### (S)—N-Benzyl-2-(2-(methoxymethyl)pyrrolidin-1-yl)quinazolin-4-amine (Compound 178)

N-Benzyl-2-chloroquinazolin-4-amine (81 mg, 0.30 mmol) and (S)-(+)-2-(methoxymethyl)pyrrolidine (37 μL, 1 eq.) were dissolved in MeCN (2 mL) and the mixture was heated to 180° C. for 10 min under microwave irradiation. After cooling, the precipitate was isolated by filtration, and washed with MeCN. The solid was then partitioned between EtOAc and aqueous NaHCO₃ solution. The organic layer was separated, dried (MgSO₄) and filtered. The crude solution was then filtered through a pad of silica, eluting with EtOAc. Concentration of the eluent gave the product as a white solid (50 mg, 48%).

LCMS RT=1.57 min, MH⁺ 349.2; ¹H NMR (DMSO-d₆): 8.93 (1H, br s), 8.11 (1H, d, J 8.0), 7.57 (1H, t, J 7.4), 7.45-7.08 (7H, br m), 4.90-4.74 (1H, br m), 4.72-4.61 (1H, br m), 4.24 (1H, br s), 3.58-3.04 (7H, br m—obscured by water signal) and 2.01-1.81 (4H, br m).

The following compounds were prepared in a similar manner, purifying by crystallisation or column chromatography where necessary:

### 2-(4,4-Difluoropiperidin-1-yl)-N-(4-fluorobenzyl)quinazolin-4-amine (Compound 137)

LCMS RT=4.63 min, MH⁺ 373.1; ¹H NMR (DMSO): 8.74-8.63 (1H, br m), 8.05 (1H, d, J 8.4), 7.53 (1H, t, J 7.7), 7.45-7.36 (2H, br m), 7.30 (1H, d, J 8.2), 7.18-7.07 (3H, br m), 4.66 (2H, d, J 5.9), 3.91-3.82 (4H, br m) and 1.94-1.76 (4H, br m).

### N-(3-Phenylpentan-3-yl)-2-(piperidin-1-yl)quinazolin-4-amine (Compound 138)

LCMS RT=4.72 min, MH⁺ 375.3; ¹H NMR (DMSO): 8.30 (1H, d, J 8.4), 7.48 (1H, td, J 7.7 and 1.3), 7.36-7.26 (3H, br m), 7.26-7.16 (3H, br m), 7.15-7.02 (2H, br m), 3.33 (4H, br m—obscured by water signal), 2.46-2.36 (2H, br m—obscured by NMR solvent signal), 2.04-1.90 (2H, br m), 1.50-1.38 (2H, br m), 1.19-1.07 (4H, br m) and 0.69 (6H, t, J 7.4).

### 2-(Piperidin-1-yl)-N-(4-(trifluoromethoxy)benzyl)quinazolin-4-amine (Compound 162)

LCMS RT=4.22 min, MH⁺ 403.1; ¹H NMR (DMSO): 8.56 (1H, br s), 8.00 (1H, d, J 8.2), 7.52-7.45 (3H, br s), 7.33-7.22 (3H, br s), 7.05 (1H, td, J 7.6 and 1.2), 4.68 (2H, d, J 5.8), 3.72-3.65 (4H, br m), 1.61-1.51 (2H, br s) and 1.43-1.33 (4H, br m).

### 4-((2-(Piperidin-1-yl)quinazolin-4-ylamino)methyl)phenol (Compound 164)

LCMS RT=4.48 min, MH⁺ 335.2; ¹H NMR (DMSO): 9.31 (1H, s), 8.46 (1H, br s), 8.04 (1H, dd, J 8.2 and 0.9), 7.53 (1H, td, J 7.6 and 1.4), 7.31-7.21 (3H, br m), 7.08 (1H, td, J 7.5 and 1.2), 6.74 (2H, d, J 8.5), 4.62 (2H, d, J 5.9), 3.85-3.79 (4H, br m), 1.71-1.61 (2H, br m) and 1.57-1.48 (4H, br m).

### 4-((2-(4-Fluorobenzylamino)quinazolin-4-ylamino)methyl)phenol (Compound 165)

LCMS RT=4.04 min, MH⁺ 385.1; ¹H NMR (DMSO): 9.25 (1H, s), 8.35 (1H, br s), 8.00 (1H, d, J 8.3), 7.48 (1H, td, J 7.6 and 1.2), 7.34 (2H, br s), 7.26-6.99 (7H, br m), 6.68 (2H, d, J 8.4), 4.60 (2H, d, J 5.4), 4.51 (2H, d, J 6.2).

### N-(4-Fluorobenzyl)-2-morpholinoquinazolin-4-amine (compound 169)

LCMS RT=1.51 min, MH⁺ 339.2; ¹H NMR (DMSO): 8.63 (1H, br s), 8.04 (1H, d, J 8.4), 7.52 (1H, td, J 7.6 and 1.4), 7.44-7.36 (2H, br s), 7.28 (1H, d, J 8.4), 7.17-7.06 (3H, br m), 4.67 (2H, d, J 5.7) and 3.72-3.54 (8H, br m).

### N⁴-(4-Fluorobenzyl)-N²-((5-methylfuran-2-yl)methyl)quinazoline-2,4-diamine (Compound 170)

LCMS RT=1.44 min, MH⁺ 363.1; ¹H NMR (DMSO): 8.46 (1H, br s), 7.99 (1H, d, J 8.2), 7.49 (1H, t, J 7.8), 7.44-7.35 (2H, br m), 7.25 (1H, d, J 8.5), 7.15-7.01 (3H, br m), 6.87 (1H, br s), 6.01 (1H, br s), 5.92 (1H, s), 4.69 (2H, d, J 5.4), 4.42 (2H, d, J 5.8) and 2.20 (3H, s).

### N²-(Benzo[b]thiophen-2-ylmethyl)-N⁴-(4-fluorobenzyl)quinazoline-2,4-diamine (Compound 171)

LCMS RT=1.50 min, MH⁺ 415.1; ¹H NMR (DMSO): 8.54 (1H, br s), 8.03 (1H, d, J 8.2), 7.84 (1H, d, J 7.8), 7.73 (1H, d, J 7.5), 7.53 (1H, td, J 7.7 and 1.3), 7.47-6.81 (10H, br m) and 4.83-4.67 (4H, br m—2 overlapping CH₂ signals).

### N-(4-Fluorobenzyl)-2-(pyrrolidin-1-yl)quinazolin-4-amine (Compound 172)

LCMS RT=1.41 min, MH⁺ 323.2; ¹H NMR (DMSO): 8.50 (1H, br s), 7.98 (1H, d, J 8.2), 7.50-7.38 (3H, br m), 7.25 (1H, d, J 8.5), 7.13 (2H, t, J 8.8), 7.02 (1H, t, J 7.5), 4.67 (2H, d, J 5.8), 3.51-3.43 (4H, br m) and 1.90-1.83 (4H, br m).

### (S)-(1-(4-(Benzylamino)quinazolin-2-yl)pyrrolidin-2-yl)methanol (Compound 177)

LCMS RT=1.50 min, MH⁺ 335.2; ¹H NMR (DMSO-d6): 7.96 (1H, d, J 8.2), 7.53 (1H, br m), 7.42-7.15 (6H, br m), 7.08 (1H, t, J 7.6), 4.72-4.52 (2H, br m), 4.48-4.36 (1H, br m), 3.65-3.32 (4H, br m—obscured by water signal) and 1.96-1.84 (4H, br m).

### (S)-Methyl 1-(4-(benzylamino)quinazolin-2-yl)pyrrolidine-2-carboxylate (Compound 180)

LCMS RT=1.36 min, MH⁺ 363.1; ¹H NMR (DMSO-d₆/D₂O at 60° C.): 7.95 (1H, d, J 8.2), 7.51 (1H, td, J 7.6 and 1.4), 7.36-7.16 (6H, br m), 7.07 (1H, td, J 7.6 and 1.1), 4.71-4.56 (2H, m), 4.48-4.42 (1H, m), 3.66-3.32 (5H, br m—obscured by water signal), 2.29-1.84 (1H, br m) and 1.98-1.84 (3H, br m).

### 5-Chloro-N-(4-fluorobenzyl)-2-(pyrrolidin-1-yl)quinazolin-4-amine (Compound 182)

LCMS RT=1.43 min, MH⁺ 357.1; ¹H NMR (DMSO): 8.32 (1H, br s), 7.48-7.35 (3H, br s), 7.21 (1H, dd, J 8.5 and 1.2), 7.13 (2H, t, J 8.9), 7.05 (1H, dd, J 7.6 and 1.2), 4.72 (2H, d, J 5.8), 3.47-3.39 (4H, br m) and 1.90-1.82 (4H, br m).

### 5-Chloro-N-(4-fluorobenzyl)-2-(piperidin-1-yl)quinazolin-4-amine (Compound 183)

LCMS RT=1.50 min, MH⁺ 371.1; ¹H NMR (DMSO): 8.32 (1H, br s), 7.45-7.37 (3H, br m), 7.20 (1H, dd, J 8.5 and 1.2), 7.13 (2H, t, J 9.0), 7.07 (1H, dd, J 7.5 and 1.2), 4.72 (2H, d, J 5.7), 3.71-3.63 (4H, br m), 1.63-1.52 (2H, br m) and 1.45-1.34 (4H, br m).

### Ethyl 2-(4-(4-fluorobenzylamino)quinazolin-2-ylamino)acetate hydrochloride (Compound 206)

LCMS RT=1.59 min; MH⁺ 355.2; ¹H NMR (DMSO): 12.98 (1H, br s), 10.32 (1H, br s), 8.40-8.25 (2H, m), 7.87-7.79 (1H, m), 7.58-7.35 (4H, m), 7.22-7.10 (2H, m), 4.79-4.70 (2H, m), 4.22-3.98 (4H, m) and 1.18-1.07 (3H, m).

### N⁴-(4-Fluorobenzyl)-N²-(2-(piperidin-1-yl)ethyl)quinazoline-2,4-diamine hydrochloride (Compound 209)

LCMS RT=1.38 min; MH⁺ 380.2; ¹H NMR (DMSO+D₂O): 7.96-7.91 (1H, m), 7.53 (1H, ddd, J 8.4, 7.1 and 1.3), 7.41-7.23 (3H, m), 7.14-7.03 (3H, m), 4.64 (2H, s), 3.50-3.39 (2H, m), 2.87-2.56 (6H, m) and 1.62-1.32 (6H, m).

### N-(1-(4-Fluorophenyl)cyclopropyl)-2-(piperidin-1-yl)quinazolin-4-amine (Compound 152)

LCMS RT=4.60 min, MH⁺ 363.2; ¹H NMR (DMSO): 8.68 (1H, br s), 8.07 (1H, d, J 8.2), 7.50 (1H, td, J 7.7 and 1.3), 7.36-7.29 (2H, br m), 7.25 (1H, d, J 8.4), 7.12-7.02 (3H, br m), 3.70-3.62 (4H, br m), 1.62-1.51 (2H, br m) and 1.41 (8H, br m).

### 2-(4,4-Difluoropiperidin-1-yl)-N-(1-(4-fluorophenyl)cyclopropyl)quinazolin-4-amine (Compound 153)

LCMS RT=4.54 min, MH⁺ 399.2; ¹H NMR (DMSO): 8.89 (1H, br s), 8.18 (1H, d, J 8.2), 7.60 (1H, td, J 7.7 and 1.3), 7.41-7.33 (3H, br m), 7.23-7.08 (3H, br m), 3.90-3.79 (4H, br m), 1.91-1.70 (4H, br m), 1.46-1.28 (4H, br m).

### N²-(4-Fluorobenzyl)-N⁴-(1-(4-fluorophenyl)cyclopropyl)quinazoline-2,4-diamine (Compound 154)

LCMS RT=4.62 min, MH⁺ 403.2; ¹H NMR (DMSO): 8.61 (1H, br s), 8.06 (1H, d, J 8.2), 7.48 (1H, Y, J 7.8 and 1.3), 7.40 (11H, br m), 4.50-4.27 (2H, br m), 1.33-1.15 (4H, br m).

### 7-Fluoro-N-(4-fluorobenzyl)-2-(piperidin-1-yl)quinazolin-4-amine (Compound 155)

LCMS RT=4.56 min, MH⁺ 355.2; ¹H NMR (DMSO): 8.60 (1H, t, J 5.7), 8.12-8.04 (1H, br m), 7.46-7.37 (2H, br m), 7.19-7.10 (2H, br m), 6.97-6.87 (2H, br m), 4.66 (2H, d, J 5.7), 3.77-3.70 (4H, br m), 1.65-1.55 (2H, br m) and 1.49-1.38 (4H, br m).

### 2-(4,4-Difluoropiperidin-1-yl)-7-fluoro-N-(4-fluorobenzyl)quinazolin-4-amine (Compound 156)

LCMS RT=4.56 min, MH⁺ 391.2; ¹H NMR (DMSO):): 8.64 (1H, t, J 5.8), 8.16-8.08 (1H, br m), 7.45-7.35 (2H, br m), 7.18-7.08 (2H, br m), 7.03-6.94 (2H, br m), 4.65 (2H, d, J 5.7), 3.91-9-3.82 (4H, br m) and 1.95-1.73 (4H, br m).

### 7-Fluoro-N², N⁴-bis(4-fluorobenzyl)quinazoline-2,4-diamine (Compound 157)

LCMS RT=4.54 min, MH⁺ 395.1; ¹H NMR (DMSO): 8.50 (1H, br s), 8.10-8.02 (1H, br m 7.48-6.80 (11H, br m), 4.72-4.57 (2H, br m) and 4.55-4.39 (2H, br m).

### (S)-(1-(4-(4-Fluorobenzylamino)quinazolin-2-yl)pyrrolidin-2-yl)methanol (Compound 198)

LCMS RT=1.55 min; MH⁺ 353.2; ¹H NMR (DMSO+D₂O): 7.89 (1H, dd, J 8.2 and 1.0), 7.50 (1H, ddd, J 8.4, 7.0 and 1.4), 7.41-7.34 (2H, m), 7.30-7.23 (1H, br m), 7.11-7.02 (3H, m), 4.61 (2H, br s), 4.15-4.06 (1H, m, obsc. by water signal), 3.58-3.18 (4H, m) and 1.95-1.69 (4H, m).

### (S)—N-(4-Fluorobenzyl)-2-(2-(methoxymethyl)pyrrolidin-1-yl)quinazolin-4-amine (Compound 199)

LCMS RT=1.62 min; MH⁺ 367.2; ¹H NMR (DMSO): 8.53 (1H, t, J 5.9), 8.01 (1H, d, J 7.7), 7.49 (1H, ddd, J 8.3, 7.0 and 1.3), 7.43-7.32 (2H, m), 7.27 (1H, dd, J 8.4 and 0.8), 7.17-7.09 (2H, m), 7.08-7.01 (1H, m), 4.82-4.69 (1H, m), 4.61 (1H, dd, J 15.2 and 5.7), 4.18 (1H, br s), 3.50-3.29 (3H, m, obsc. By water signal), 3.17-2.98 (4H, m) and 1.97-1.77 (4H, m).

### (S)-Methyl 1-(4-(4-fluorobenzylamino)quinazolin-2-yl)pyrrolidine-2-carboxylate (Compound 200)

LCMS RT=1.59 min; MH⁺ 381.1; ¹H NMR (DMSO): 8.62-8.49 (1H, m), 8.00 (1H, d, J 7.6), 7.56-7.27 (4H, m), 7.19-7.02 (3H, m), 4.73-4.55 (2H, m), 4.50-4.38 (1H, m), 3.76-3.55 (3H, m), 3.51-3.43 (3H, m), 2.33-2.19 (1H, m) and 1.98-1.74 (3H, m).

### 2-(4-Benzylpiperazin-1-yl)-N-(4-fluorobenzyl)quinazolin-4-amine (Compound 201)

LCMS RT=1.42 min; MH⁺ 428.0; ¹H NMR (DMSO): 8.60-8.53 (1H, m), 8.00 (1H, d, J 8.2), 7.49 (1H, t, J 7.6), 7.43-7.21 (8H, m), 7.16-7.02 (3H, m), 4.63 (2H, d, J 5.5), 3.77-3.66 (4H, m), 3.47 (2H, s) and 2.38-2.29 (4H, m).

### Cyclohexyl(4-(4-(4-fluorobenzylamino)quinazolin-2-yl)piperazin-1-yl)methanone (Compound 203)

LCMS RT=1.66 min; MH⁺ 448.3; ¹H NMR (DMSO): 8.63 (1H, t, J 5.8), 8.03 (1H, d, J 8.1), 7.55-7.48 (1H, m), 7.46-7.37 (2H, m), 7.28 (1H, d, J 8.2), 7.18-7.05 (3H, m), 4.67 (2H, d, J 5.8), 3.80-3.62 (4H, m), 3.52-3.40 (4H, m), 2.65-2.51 (1H, m, obsc. by DMSO signal) 1.74-1.57 (5H, m) and 1.41-1.10 (5H, m).

### (4-(4-(4-Fluorobenzylamino)quinazolin-2-yl)piperazin-1-yl)(phenyl)methanone (Compound 204)

LCMS RT=1.61 min; MH⁺ 442.2; ¹H NMR (DMSO): 8.65 (1H, t, J 5.7), 8.04 (1H, d, J 7.8), 7.56-7.36 (8H, m), 7.28 (1H, d, J 7.9), 7.16-7.06 (3H, m), 4.66 (2H, d, J 5.6), 3.89-3.50 (6H, m) and 3.44-4.31 (2H, m, obsc. by DMSO peak).

### N⁴-(4-Fluorobenzyl)-N²-(2-methoxyethyl)quinazoline-2,4-diamine (Compound 207)

LCMS RT=1.54 min; MH⁺ 327.2; ¹H NMR (DMSO+D₂O): 7.89 (1H, d, J 8.3), 7.54-7.46 (1H, m), 7.40-7.32 (2H, m), 7.28-7.20 (1H, m), 7.11-7.02 (3H, m), 4.62 (2H, br s), 3.43-3.26 (4H, m) and 3.17 (3H, s).

### N²-(Cyclohexylmethyl)-N⁴-(4-fluorobenzyl)quinazoline-2,4-diamine (Compound 208)

LCMS RT=1.70 min; MH⁺ 365.2; ¹H NMR (DMSO+D₂O): 7.88 (1H, d, J 8.1), 7.48 (1H, ddd, J 8.3, 7.0 and 1.3), 7.39-7.28 (2H, m), 7.27-7.14 (1H, m), 7.11-6.99 (3H, m), 4.62 (2H, s), 3.04 (2H, d, J 6.8), 1.63-1.19 (6H, m), 1.09-0.91 (3H, m) and 0.83-0.59 (2H, m).

### Method 3

### Compounds of General Formula (VII)

### 4-Chloro-2-(piperidin-1-yl)quinazoline

2,4-Dichloroquinazoline (1.5 g, 7.5 mmol) was dissolved in 1,4-dioxane (15 mL). N-Methylpiperidine (961 □L, 7.9 mmol) was added and the mixture was heated to 150° C. for 5 min under microwave irradiation. The mixture was diluted with EtOAc and washed with saturated aqueous NaHCO₃ and brine (×2). The organic layer was dried (MgSO₄), filtered and concentrated in vacuo. The crude product was purified by column chromatography (5% EtOAc-petrol) to afford the title compound as a yellow oil that solidified on evaporation from petrol to a yellow solid (1.33 g, 72%).

¹H NMR (DMSO): 7.95-7.91 (1H, m), 7.77 (1H, ddd, J 8.5, 7.0 and 1.4), 7.54-7.49 (1H, m), 7.32 (1H, ddd, J 8.1, 7.0 and 1.1), 3.84-3.79 (4H, m) and 1.69-1.51 (6H, m).

### Method 4a

### Compounds of General Formula (I)

### (S)—N-(1-Phenylethyl)-2-(piperidin-1-yl)quinazolin-4-amine (Compound 32)

4-Chloro-2-(piperidin-1-yl)quinazoline (50 mg, 0.20 mmol) was suspended in IPA (Solvent S, 2 mL), and treated successively with NEt₃ (56 □L, 0.40 mmol, 2 eq. EB) and S-(−)-□-phenethylamine (27 □L, 0.21 mmol, 1.05 eq. E). The mixture was heated to 180° C. (Temperature K) for 20 minutes (Time T) under microwave irradiation. If after cooling SM remained, further S-(−)-□-phenethylamine (14 □L, 0.10 mmol) was added and the mixture again heated to 180° C. for 20 minutes under microwave irradiation. After cooling to room temperature, the crude reaction mixture was concentrated, then redissolved in EtOAc. The solution was washed with saturated aqueous NaHCO₃, saturated aqueous NH₄Cl and brine, then dried (MgSO₄), filtered and concentrated. The crude product was purified by column chromatography (4:1 petrol-EtOAc to 1:1 petrol-EtOAc) to afford the title compound as an off-white solid (39 mg, 59%).

LCMS RT=9.12 min, MH⁺ 333.2; ¹H NMR (DMSO): 8.18-8.10 (2H, m), 7.50-7.39 (3H, m), 7.31-7.24 (2H, m), 7.23-7.15 (2H, m), 7.07-7.01 (1H, m), 5.35 (1H, qn, J 7.0), 3.71-3.64 (4H, m), 1.59-1.53 (5H, m) and 1.46-1.27 (4H, m).

The following compounds were prepared in a similar manner, purifying by crystallisation or column chromatography where necessary:

### N-Phenethyl-2-(piperidin-1-yl)quinazolin-4-amine (Compound 33)

LCMS RT=10.0 min, MH⁺ 333.2; ¹H NMR (DMSO): 8.07-8.02 (1H, m), 7.92 (1H, dd, J 8.2 and 1.1), 7.47 (1H, ddd, J 8.5, 7.1 and 1.4), 7.34-7.17 (6H, m), 7.01 (1H, ddd, J 8.0, 7.0 and 1.2), 3.84-3.78 (4H, m), 3.70-3.61 (2H, m), 2.99-2.92 (2H, m), 1.67-1.59 (2H, m) and 1.58-1.47 (4H, m).

### N-(4-Chlorobenzyl)-2-(piperidin-1-yl)quinazolin-4-amine (Compound 34)

LCMS RT=9.10 min, MH⁺ 353.1; ¹H NMR (DMSO): 8.54 (1H, t, J 6.0), 7.98 (1H, d, J 7.58), 7.48 (1H, ddd, J 8.3, 7.0 and 1.2), 7.40-7.33 (4H, m), 7.25-7.22 (1H, d, J 7.6), 7.04 (1H, ddd, J 8.0, 7.0 and 1.0), 4.64 (2H, d, J 5.7), 3.69 (4H, t, J 5.3), 1.61-1.54 (2H, m) and 1.44-1.37 (4H, m).

### N-(Naphthalen-1-ylmethyl)-2-(piperidin-1-yl)quinazolin-4-amine (Compound 37)

LCMS RT=9.79 min, MH⁺ 369.3; ¹H NMR (DMSO): 8.49 (1H, t, J 5.2), 8.30 (1H, dd, J 7.1 and 2.2), 8.05 (1H, d, J 7.3), 7.96-7.91 (1H, m), 7.83 (1H, d, J 8.1), 7.58-7.43 (5H, m), 7.24 (1H, dd, J 8.4 and 0.8), 7.02 (1H, ddd, J 8.1, 7.0 and 1.1), 5.16 (2H, d, J 5.4), 3.69 (4H, t, J 5.4), 1.60-1.51 (2H, m) and 1.44-1.34 (4H, m).

### 2-(Piperidin-1-yl)-N-(4-(trifluoromethyl)benzyl)quinazolin-4-amine (Compound 38)

LCMS RT=8.82 min, MH⁺ 387.1; ¹H NMR (DMSO): 8.67-8.59 (1H, m), 8.00 (1H, br d), 7.67 (2H, app. d), 7.56 (2H, app. d), 7.53-7.46 (1H, m), 7.25 (1H, br d), 7.09-7.03 (1H, m), 4.74 (2H, d, J 5.6), 3.69-3.63 (4H, m), 1.60-1.50 (2H, m) and 1.42-1.30 94H, m).

### (R)—N-(1-Phenylethyl)-2-(piperidin-1-yl)quinazolin-4-amine (Compound 39)

LCMS RT=9.16 min, MH⁺ 333.2; ¹H NMR (DMSO): 8.18-8.10 (2H, m), 7.50-7.39 (3H, m), 7.31-7.24 (2H, m), 7.23-7.15 (2H, m), 7.07-7.01 (1H, m), 5.35 (1H, qn, J 7.0), 3.71-3.64 (4H, m), 1.59-1.53 (5H, m) and 1.46-1.27 (4H, m).

### N-(2-Chlorobenzyl)-2-(piperidin-1-yl)quinazolin-4-amine (Compound 40)

LCMS RT=9.38 min, MH⁺ 353.1; ¹H NMR (DMSO): 8.56-8.52 (1H, m), 8.05 (1H, dd, J 8.2 and 1.0), 7.53-7.41 (2H, m), 7.38-7.32 (1H, m), 7.29-7.23 (3H, m), 7.07 (1H, ddd, J 8.0, 6.9 and 1.1), 4.75 (2H, d, J 5.7), 3.68-3.63 (4H, m), 1.60-1.49 (2H, m) and 1.43-1.31 (4H, m).

### N-(3-Chlorobenzyl)-2-(piperidin-1-yl)quinazolin-4-amine (Compound 41)

LCMS RT=9.27 min, MH⁺ 353.1; ¹H NMR (DMSO): 8.58-8.54 (1H, m), 7.98 (1H, dd, J 8.2 and 1.1), 7.49 (1H, ddd, J 8.4, 6.9 and 1.2), 7.44-7.42 (1H, m), 7.35-7.23 (4H, m), 7.05 (1 H, ddd, J 8.0, 7.1 and 1.2), 4.65 (2H, d, J 5.9), 3.74-3.68 (4H, m), 1.62-1.53 (2H, m) and 1.46-1.36 (4H, m).

### 2-(Piperidin-1-yl)-N-(2-(trifluoromethyl)benzyl)quinazolin-4-amine (Compound 42)

LCMS RT=9.22 min, MH⁺ 387.1; ¹H NMR (DMSO): 8.63-8.59 (1H, m), 8.08 (1H, dd, J 8.2 and 1.1), 7.73 (1H, br d), 7.61-7.40 (4H, m), 7.27 (1H, dd, J 8.4 and 0.9), 7.09 (1H, ddd, J 8.9, 6.1 and 1.10), 4.86 (2H, d, J 6.0), 3.63-3.55 (4H, m), 1.56-1.47 (2H, m) and 1.36-1.26 (4H, m).

### N-(4-Methylbenzyl)-2-(piperidin-1-yl)quinazolin-4-amine (Compound 43)

LCMS RT=9.41 min, MH⁺ 333.2; ¹H NMR (DMSO): 8.47 (1H, t, J 6.3), 7.99 (1H, dd, J 8.3 and 1.1), 7.47 (1H, ddd, J 8.2, 6.8 and 1.3), 7.26 (2H, d, J 8.1), 7.23 (1H, d, J 7.9), 7.10 (2H, d, J 7.8), 7.02 (1H, ddd, J 8.1, 7.0 and 1.2), 4.62 (2H, d, J 5.8), 3.73 (4H, t, J 5.3), 2.25 (3H, s), 1.63-1.53 (2H, m) and 1.49-1.39 (4H, m).

### N-(4-Methoxybenzyl)-2-(piperidin-1-yl)quinazolin-4-amine (Compound 44)

LCMS RT=8.38 min, MH⁺ 349.2; ¹H NMR (DMSO): 8.47 (1H, t, J 5.8), 7.98 (1H, dd, J 8.2 and 1.1), 7.47 (1H, ddd, J 8.3, 7.0 and 1.2), 7.30 (2H, d, J 8.7), 7.23 (1H, dd, J 8.4 and 0.8), 7.02 (1H, ddd, J 8.1, 7.1 and 1.1), 6.86 (2H, d, J 8.7), 4.61 (2H, d, J 5.9), 3.75 (4H, t, J 5.3), 3.70 (3H, s), 1.64-1.55 (2H, m) and 1.50-1.40 (4H, m).

### N-(Cyclohexylmethyl)-2-(piperidin-1-yl)quinazolin-4-amine (Compound 45)

LCMS MH⁺ 325.3; ¹H NMR (DMSO): 7.97 (1H, dd, J 8.1 and 0.8), 7.89 (1H, t, J 5.6); 7.45 (1H, ddd, J 8.3, 6.9 and 1.4), 7.22 (1H, dd, J 8.4 and 0.9), 7.00 (1H, ddd, J 8.1, 6.9 and 1.2), 3.77 (4H, t, J 5.4), 3.70 (3H, s), 1.79-1.56 (8H, m), 1.54-1.44 (4H, m) 1.24-1.10 (3H, m) and 1.04-0.89 (2H, m).

### 2-(Piperidin-1-yl)-N-(3-(trifluoromethyl)benzyl)quinazolin-4-amine (Compound 49)

LCMS RT=8.97 min, MH⁺ 387.1; ¹H NMR (DMSO): 8.72 (1H, br s), 8.01 (1H, d, J 8.0), 7.74 (1H, s), 7.70-7.65 (1H, m), 7.62-7.48 (3H, m), 7.29-7.25 (1H, m), 7.12-7.06 (1H, m), 4.73 (2H, d, J 5.9), 3.72-3.65 (4H, m), 1.63-1.52 (2H, m) and 1.45-1.34 (4H, m).

### N-(3-Fluorobenzyl)-2-(piperidin-1-yl)quinazolin-4-amine (Compound 64)

LCMS RT=8.43 min, MH⁺ 337.2; ¹H NMR (DMSO): 8.55 (1H, t, J 5.9), 8.00 (1H, dd, J 8.2 and 1.0), 7.49 (1H, ddd, J 8.4, 6.9 and 1.4), 7.38-7.31 (1H, m), 7.26-7.15 (3H, m), 7.08-7.00 (2H, m), 4.67 (2H, d, J 5.67), 3.72-3.69 (4H, m), 1.61-1.53 (2H, m) and 1.44-1.37 (4H, m).

### N-(4-Fluorobenzyl)-2-(piperidin-1-yl)quinazolin-4-amine (Compound 63)

LCMS RT=8.36 min, MH⁺ 337.2; ¹H NMR (DMSO): 8.52 (1H, t, J 6.0), 7.99 (1H, dd, J 8.3 and 1.1), 7.48 (1H, ddd, J 8.4, 6.9 and 1.5), 7.43-7.37 (2H, m), 7.23 (1H, dd, J 8.4 and 0.8), 7.16-7.08 (2H, m), 7.04 (1H, ddd, J 8.1, 6.9 and 1.2), 4.65 (2H, d, J 5.8), 3.73-3.70 (4H, m), 1.62-1.55 (2H, m) and 1.46-1.38 (4H, m).

### N-(2-Fluorobenzyl)-2-(piperidin-1-yl)quinazolin-4-amine (Compound 65)

LCMS RT=8.62 min, MH⁺ 337.2; ¹H NMR (DMSO): 8.50 (1H, t, J 5.6), 8.02 (1H, dd, J 8.2 and 1.1), 7.49 (1H, ddd, J 8.5, 6.9 and 1.5), 7.39 (1H, td, J 7.7 and 1.8), 7.31-7.09 (3H, m), 7.05 (1H, ddd, J 8.9, 6.9 and 1.2), 4.71 (2H, d, J 5.7), 3.72-3.68 (4H, m), 1.61-1.53 (2H, m) and 1.44-1.36 (4H, m).

### N,N-Dibenzyl-2-(piperidin-1-yl)quinazolin-4-amine (Compound 66)

LCMS RT=11.72 min, MH⁺ 409.3; ¹H NMR (DMSO): 7.71 (1H, dd, J 8.3 and 1.0), 7.49 (1H, ddd, J 8.4, 6.9 and 1.4), 7.39-7.23 (11H, m), 6.91 (1H, ddd, J 8.3, 6.9 and 1.4), 4.85 (4H, br s), 3.67-3.63 (4H, m), 1.59-1.51 (2H, m) and 1.40-1.32 (4H, m).

### N-(2,4-Dichlorobenzyl)-2-(piperidin-1-yl)quinazolin-4-amine (Compound 69)

LCMS RT=9.86 min, MH⁺ not found; ¹H NMR (DMSO): 8.57 (1H, t, J 5.6), 8.03 (1H, dd, J 8.3 and 1.1), 7.61 (1H, t, J 1.1), 7.50 (1H, td, J 7.7 and 1.4), 7.35 (2H, d, J 1.2), 7.26 (1H, dd, J 8.5 and 0.8), 7.07 (1H, td, J 7.5 and 1.2), 4.71 (2H, d, J 5.7), 3.69-3.59 (4H, br m), 1.60-1.49 (2H, br m) and 1.42-1.30 (4H, br m).

### N-(3,4-Dichlorobenzyl)-2-(piperidin-1-yl)quinazolin-4-amine (Compound 68)

LCMS RT=9.51 min, MH⁺ 387.1; ¹H NMR (DMSO): 8.57 (1H, t, J 5.8), 7.97 (1H, dd, J 8.2 and 1.1), 7.62 (1H, d, J 2.0), 7.56 (1H, d, J 8.2), 7.49 (1H, td, J 7.6 and 1.4), 7.34 (1H, dd, J 8.5 and 2.0), 7.24 (1H, dd, J 8.5 and 0.9), 7.05 (1H, td, J 7.6 and 1.1), 4.63 (2H, d, J 5.9), 3.73-3.64 (4H, br m), 1.62-1.52 (2H, br m) and 1.45-1.34 (4H, br m).

### Method 4b

### Compounds of General Formula (I)

### N-Benzyl-N-methyl-2-(piperidin-1-yl)quinazolin-4-amine (Compound 35)

4-Chloro-2-(piperidin-1-yl)quinazoline (99 mg, 0.4 mmol) was dissolved in dry pyridine (Solvent S, 1.5 mL), and treated with N-methylbenzylamine (54.2 □L, 0.42 mmol, 1.05 eq E). The mixture was heated to 200° C. (Temperature K) for 10 minutes (Time T) under microwave irradiation. After cooling to room temperature, the crude reaction mixture diluted with EtOAc. The solution was washed with saturated aqueous CuSO₄ (3×10 mL) and brine, then dried (MgSO₄), filtered and concentrated. The crude product was purified by column chromatography (4:1 petrol-EtOAc to 1:1 petrol-EtOAc) to afford the title compound as pale yellow oil (85 mg, 64%).

LCMS RT=10.30 min, MH⁺ 332.5; ¹H NMR (DMSO): 7.79 (1H, dd, J 8.3 and 1.3), 7.48 (1H, ddd, J 8.3, 6.7 and 1.2), 7.41-7.27 (6H, m), 6.96 (1H, ddd, J 8.3, 6.9 and 1.3), 4.87 (2H, s), 3.74 (4H, t, J 5.4), 3.22 (3H, s), 1.64-1.54 (2H, m) and 1.51-1.42 (4H, m).

The following compounds were prepared in a similar manner, purifying by crystallisation or column chromatography where necessary:

### 4-(3,4-Dihydroisoquinolin-2(1H)-yl)-2-(piperidin-1-yl)quinazoline (Compound 36)

LCMS RT=11.15 min, MH⁺ 345.2; ¹H NMR (CDCl3): 7.77 (1H, d, J 8.4), 7.51 (1H, d, J 3.9), 7.23-7.19 (4H, m), 7.09-7.04 (1H, m), 4.85 (2H, s), 3.96 (2H, t, J 5.9), 3.93-3.87 (4H, m), 3.16 (2H, t, J 5.9) and 1.71-1.63 (6H, m).

### Method 4d

### Compounds of General Formula (I)

### 2-(Piperidin-1-yl)-N-(pyridin-2-ylmethyl)quinazolin-4-amine (Compound 46)

4-Chloro-2-(piperidin-1-yl)quinazoline (75 mg, 0.30 mmol) was suspended in IPA (Solvent S, 2 mL), and treated with 2-picolylamine (33 □L, 0.31 mmol, 1.05 eq. E). The mixture was heated to 180° C. (Temperature K) for 15 minutes (Time T) under microwave irradiation. After cooling to room temperature, the crude reaction mixture was diluted with water and saturated aqueous NaHCO₃ solution then extracted with EtOAc (2×15 mL). The separated organic layer was washed with brine, then dried (MgSO₄), filtered and concentrated. The crude product was purified by column chromatography (1:1 petrol-EtOAc to 100% EtOAc) to afford the title compound as an off-white solid (55 mg, 57%).

LCMS RT=6.56 min, MH⁺ 320.3; ¹H NMR (DMSO): 8.62 (1H, t, J 5.7, NH), 8.49 (1H, d, J 4.8), 8.04 (1H, d, J 7.3), 7.70 (1H, m), 7.49 (1H, ddd, J 8.3, 7.0 and 1.2), 7.31 (1H, d, J 7.8), 7.27-7.20 (2H, m), 7.06 (1H, ddd, J 8.1, 7.0 and 1.1), 4.74 (2H, d, J 5.7), 3.63 (4H, t, J 5.3), 1.59-1.49 (2H, m) and 1.40-1.29 (4H, m).

The following compounds were prepared in a similar manner, purifying by crystallisation or column chromatography where necessary:

### 2-(Piperidin-1-yl)-N-(pyridin-3-ylmethyl)quinazolin-4-amine (Compound 47)

LCMS RT=6.30 min, MH⁺ 320.3; ¹H NMR (DMSO): 8.60 (1H, d, J 1.7), 8.57 (1H, t, NH, J 5.6), 8.43 (1H, dd, J 4.7 and 1.7), 7.98 (1H, dd, J 8.2 and 1.1), 7.75 (1H, d, J 7.8), 7.48 (1H, ddd, J 8.2, 6.8 and 1.2), 7.33 (1H, ddd, J 5.5, 4.8 and 0.7), 7.24 (1H, dd, J 8.4 and 0.9), 7.05 (1H, ddd, 8.1, 7.0 and 1.2), 4.68 (2H, d, J 5.5), 3.71 (4H, t, J 5.1), 1.62-1.53 (2H, m) and 1.46-1.36 (4H, m).

### 2-(Piperidin-1-yl)-N-(thiophen-2-ylmethyl)quinazolin-4-amine (Compound 48)

LCMS RT=8.01 min, MH⁺ 325.2; ¹H NMR (DMSO): 8.56 (1H, t, J 5.7, NH), 7.93 (1H, dd, J 8.1 and 1.1), 7.48 (1H, ddd, J 8.4, 7.0 and 1.4), 7.34 (1H, dd, J 5.2 and 1.4), 7.24 (1H, dd, J 8.4 and 1.0), 7.06 (1H, dd, J 3.4 and 1.1), 7.02 (1H, ddd, J 8.1, 6.9 and 1.2), 6.94 (1 H, dd, J 5.1 and 3.4), 4.83 (2H, d, J 5.9), 3.81 (4H, t, J 5.5), 1.67-1.57 (2H, m) and 1.55-1.45 (4H, m).

### 4-((2-(Piperidin-1-yl)quinazolin-4-ylamino)methyl)benzonitrile (Compound 92)

LCMS RT=4.55 min; MH⁺=344.3; ¹H NMR (DMSO): 8.62 (1H, t, J 5.6), 8.00 (1H, d, J 8.4), 7.77 (2H, d, J 8.0), 7.54 (2H, d, J 8.0), 7.49 (1H, t, J 7.5), 7.24 (1H, d, J 8.4), 7.06 (1H, t, J 7.5), 4.72 (2H, d, J 5.6), 3.64 (4H, m), 1.61-1.49 (2H, m) and 1.42-1.31 (4H, m).

### Methyl 4-((2-(piperidin-1-yl)quinazolin-4-ylamino)methyl)benzoate (Compound 93)

LCMS RT=4.61 min; MH⁺=377.2; ¹H NMR (DMSO): 8.61 (1H, t, J 5.3), 8.01 (1H, d, J 7.1), 7.90 (2H, d, J 8.3), 7.52-7.45 (3H, m), 7.24 (1H, d, J 7.6), 7.05 (1H, ddd, J 8.0, 6.9 and 1.1), 4.73 (2H, d, J 5.8), 3.82 (3H, s), 3.66 (4H, t, J 5.2), 1.60-1.50 (2H, m), and 1.43-1.32 (4H, m).

### N-(4-(Methylsulfonyl)benzyl)-2-(piperidin-1-yl)quinazolin-4-amine (Compound 94)

LCMS RT=4.49 min; MH⁺=397.2; ¹H NMR (DMSO): 8.65 (1H, t, J 6.2), 8.00 (1H, d, J 7.5), 7.85 (2H, d, J 8.3), 7.61 (2H, d, J 8.3), 7.49 (1H, ddd, J 8.3, 6.9 and 1.4), 7.24 (1H, d, J 7.8), 7.06 (1H, ddd, J 8.1, 7.0 and 1.0), 4.74 (2H, d, J 5.4), 3.65 (4H, t, J 4.9), 3.15 (3H, s), 1.60-1.49 (2H, m) and 1.41-1.30 (4H, m).

### N-(3-Phenylpropyl)-2-(piperidin-1-yl)quinazolin-4-amine (Compound 95)

LCMS RT=4.66 min; MH⁺=347.3; ¹H NMR (DMSO): 7.94 (1H, d, J 7.5), 7.91 (1H, t, J 5.5), 7.46 (1H, ddd, J 8.3, 6.9 and 1.3), 7.32-7.14 (6H, m), 7.01 (1H, ddd, J 7.9, 6.9 and 1.1), 3.72 (4H, t, J 4.8), 3.53-3.43 (2H, m), 3.15 (3H, s), 2.67 (2H, t, J 7.0), 1.94 (2H, t, J 7.0), 1.65-1.55 (2H, m) and 1.53-1.43 (4H, m).

### (R)—N-(1-(4-Methoxyphenyl)ethyl)-2-(piperidin-1-yl)quinazolin-4-amine (Compound 97)

LCMS RT=4.62 min; MH⁺=363.3; ¹H NMR (DMSO): 8.13 (1H, dd, J 8.2 and 0.9), 8.04 (1H, d, J 7.6), 7.46 (1H, ddd, J 8.1, 7.0 and 1.1), 7.34 (2H, d, J 8.6), 7.21 (1H, dd, J 8.4 and 0.9), 7.03 (1H, ddd, J 8.1, 7.0 and 1.1), 6.84 (2H, d, J 8.7), 5.33 (1H, m), 3.77-3.64 (7H, m), 1.63-1.50 (5H, m) and 1.48-1.34 (4H, m).

### (R)-2-(Piperidin-1-yl)-N-(1-p-tolylethyl)quinazolin-4-amine (Compound 101)

LCMS RT=4.64 min; MH⁺=347.3; ¹H NMR (DMSO): 8.15 (1H, dd, J 8.1 and 0.9), 8.06 (1H, d, J 7.3), 7.46 (1H, ddd, J 8.2, 6.7 and 1.3), 7.30 (2H, d, J 8.1), 7.21 (1H, dd, J 8.4 and 0.9), 7.08 (2H, d, J 7.9), 7.03 (1H, ddd, J 8.2, 6.9 and 1.3), 5.33 (1H, m), 3.69 (4H, t, J 5.5), 2.24 (3H, s), 1.63-1.49 (5H, m) and 1.48-1.30 (4H, m).

### (R)-2-(Piperidin-1-yl)-N-(1-(4-(trifluoromethyl)phenyl)ethyl)quinazolin-4-amine (Compound 117)

LCMS RT=4.65 min, MH⁺ 401.2; ¹H NMR (DMSO): 8.27-8.14 (2H, br m), 7.63 (4H, m), 7.52-7.45 (1H, m), 7.22 (1H, dd, J 8.4 and 0.9), 7.10-7.03 (1H, m), 5.34 (1H, t, J 7.1), 3.71-3.51 (4H, br m), 1.64-1.46 (5H, br m) and 1.44-1.13 (4H, br m).

### 4-(4-Phenylpiperidin-1-yl)-2-(piperidin-1-yl)quinazoline (Compound 90)

LCMS RT=4.69 min, MH⁺ 373.3; ¹H NMR (DMSO): 7.76 (1H, d, J 8.1), 7.53 (1H, td, J 7.4 and 1.2), 7.40-7.27 (5H, br m), 7.26-7.17 (1H, br m), 7.13-7.04 (1H, td, J 7.5 and 1.2), 4.27 (2H, d, J 12.6), 3.86-3.74 (4H, br m), 3.22 (2H, br m), 2.92-2.77 (1H, br m), 1.96-1.78 (4H, br m) and 1.70-1.45 (6H, br m).

### 4-(4-(4-Chlorophenyl)piperazin-1-yl)-2-(piperidin-1-yl)quinazoline (Compound 89)

LCMS RT=4.73 min, MH⁺ 408.2; ¹H NMR (DMSO): 7.79 (1H, d, J 8.3), 7.55 (1H, t, J 7.4), 7.38 (1H, d, J 8.3), 7.27 (2H, d, J 9.0), 7.10 (1H, td, J 7.5 and 1.2), 7.00 (2H, d, J 9.0), 3.86-3.66 (8H, br m), 3.31 (4H, br m—obscured by water signal) and 1.68-1.47 (6H, br m).

### 4-(4-Phenylpiperazin-1-yl)-2-(piperidin-1-yl)quinazoline (Compound 88)

LCMS RT=4.64 min, MH⁺ 374.2; ¹H NMR (DMSO): 7.80 (1H, d, J 7.9), 7.55 (1H, td, J 7.6 and 1.4), 7.38 (1H, dd, J 8.5 and 1.0), 7.29-7.21 (2H, br m), 7.10 (1H, td, J 7.5 and 1.2), 6.99 (2H, d, J 7.8), 6.81 (1H, t, J 7.2), 3.85-3.67 (8H, br m), 3.31 (4H, br m—obscured by water signal) and 1.69-1.48 (6H, br m).

### (S)—N-(1-Methoxy-3-phenylpropan-2-yl)-2-(piperidin-1-yl)quinazolin-4-amine (Compound 144)

LCMS RT=4.52 min, MH⁺ 377.2; ¹H NMR (DMSO): 8.02 (1H, d, J 8.5), 7.62 (1H, d, J 8.3), 7.46 (1H, m), 7.31-7.17 (5H, br m), 7.17-7.09 (1H, br m), 7.05-6.98 (1H, m), 4.74-4.60 (1H, m), 3.80-3.70 (4H, br m), 3.59-3.50 (1H, br m), 3.49-3.41 (1H, br m), 3.28 (3H, s), 2.95 (2H, d, J 7.3), 1.67-1.56 (2H, br m) and 1.56-1.43 (4H, br m).

### (S)-Methyl 2-phenyl-2-(2-(piperidin-1-yl)quinazolin-4-ylamino)acetate (Compound 175)

LCMS RT=1.74 min, MH⁺ 377.1; ¹H NMR (DMSO-d₆): 8.37 (1H, br s), 8.21 (1H, d, J 8.2), 7.58-7.48 (3H, br m), 7.47-7.39 (3H, br m), 7.28 (1H, d, J 8.4), 7.03 (1H, t, J 7.6), 5.62 (1H, d, J 5.3), 3.79-3.72 (4H, br m), 3.64 (3H, s), 1.68-1.56 (2H, br m) and 1.56-1.43 (4H, br m).

### (S)-Methyl 3-phenyl-2-(2-(piperidin-1-yl)quinazolin-4-ylamino)propanoate (Compound 176)

LCMS RT=1.61 min, MH⁺ 391.1; ¹H NMR (DMSO-d₆): 8.23 (1H, d, J 6.6), 8.04 (1H, d, J 8.2), 7.50 (1H, td, J 7.7 and 1.2), 7.35-7.15 (6H, br m), 7.06 (1H, td, J 7.5 and 1.2), 4.65-4.56 (1H, br m), 3.74-3.65 (4H, br m), 3.56 (3H, s), 3.33-3.12 (2H, br m—obscured by water signal on left hand side), 1.65-1.53 (2H, br m) and 1.52-1.37 (4H, br m).

### Method 4e

### Compounds of General Formula (I)

### N-Phenyl-2-(piperidin-1-yl)quinazolin-4-amine hydrochloride (Compound 72)

4-Chloro-2-(piperidin-1-yl)quinazoline (50 mg, 0.20 mmol) was suspended in IPA (Solvent S, 2 mL), and treated successively with NEt₃ (84.5 □L, 0.61 mmol, 3 eq. EB) and aniline (19 □L, 0.21 mmol, 1.05 eq. E). The mixture was heated to 150° C. (Temperature K) for 2×10 minutes (Time T) under microwave irradiation. After cooling to room temperature the crude reaction mixture diluted with EtOAc. The solution was washed with saturated aqueous NaHCO₃, saturated aqueous NH₄Cl and brine, then dried (MgSO₄), filtered and concentrated. The crude product formed crystals on standing, which were collected by suspending in petrol and filtering. The crystals were washed with petrol and dried under suction to afford the title compound as white crystals solid (20 mg, 32%).

LCMS RT=8.07 min, MH⁺ 305.2; ¹H NMR (DMSO): 11.96 (1H, br s), 10.79 (1H, br s), 8.53-8.50 (1H, m), 7.90-7.76 (2H, m), 7.71-7.66 (2H, m), 7.55-7.44 (3H, m), 7.32-7.26 (1H, m), 3.81-3.75 (4H, m) and 1.70-1.57 (6H, m).

### Method 4f

### Compounds of General Formula (I)

### N-(3-Methoxybenzyl)-2-(piperidin-1-yl)quinazolin-4-amine hydrochloride (Compound 82)

4-Chloro-2-(piperidin-1-yl)quinazoline (100 mg, 0.40 mmol) was suspended in MeCN (Solvent S, 2 mL), and treated with 3-methoxybenzylamine (55 □L, 0.42 mmol, 1.05 eq. E). The mixture was heated to 200° C. (Temperature K) for 10 minutes (Time T) under microwave irradiation. After cooling to room temperature, a white precipitate formed which was filtered and washed with MeCN (3×10 mL). The product was dried under vacuum to afford the title compound as a white solid (77 mg, 50%).

LCMS RT=4.60 min, MH⁺ 349.2; ¹H NMR (DMSO): 11.90 (1H, br s), 10.14 (1H, br s), 8.34 (1H, d, J 8.2), 7.81 (2H, d, J 4.0), 7.49-7.38 (1H, br m), 7.25 (1H, t, J 7.9), 6.97 (2H, d, J 8.0), 6.84 (1H, ddd, J 8.3, 2.5 and 1.0), 4.73 (2H, d, J 5.7), 3.89-3.77 (4H, br m), 3.73 (3H, s) and 1.70-1.49 (6H; br m).

Reactions that did not go to completion were resubjected to the reaction conditions as indicated in Table 1. If no precipitate formed on cooling, the mixture could be concentrated under reduced pressure and suspended in EtOAc before filtering. EtOAc was used to wash the products in these cases, and could also be used in place of MeCN for washing in other cases.

The following compounds were prepared in a similar manner, purifying by crystallisation or column chromatography where necessary:

### (R)—N-(1-(4-Chlorophenyl)ethyl)-2-(piperidin-1-yl)quinazolin-4-amine hydrochloride (Compound 96)

LCMS RT=4.69 min MH⁺=367.2; ¹H NMR (DMSO): 11.9 (1H, s, HCl), 9.66 (1H, d, J 5.6), 8.51 (1H, d, J 8.0), 7.84-7.76 (2H, m), 7.51-7.36 (5H, m), 5.42 (1H, m), 3.78 (4H, s), 1.62 (3H, d, J 7.0) and 1.60-1.39 (6H, m).

### (R)—N-(1-(4-Fluorophenyl)ethyl)-2-(piperidin-1-yl)quinazolin-4-amine hydrochloride (Compound 98)

LCMS RT=4.64 min; MH⁺=351.2; ¹H NMR (DMSO): 11.9 (1H, s, HCl), 9.66 (1H, d, J 5.4), 8.51 (1H, d, J 8.1), 7.79 (2H, d, J 3.8), 7.53-7.40 (3H, m), 7.16 (1H, t, J 8.9), 5.45 (1H, m), 3.79 (4H, s), 1.62 (3H, d, J 6.7) and 1.60-1.38 (6H, m).

### (R)—N-(1-(Naphthalen-1-yl)ethyl)-2-(piperidin-1-yl)quinazolin-4-amine hydrochloride (Compound 99)

LCMS RT=4.69 min; MH⁺=383.2; ¹H NMR (DMSO): 11.83 (1H, s, HCl), 9.87 (1H, d, J 6.5), 8.58 (1H, d, J 8.6), 8.30 (1H, d, J 8.6), 7.96 (1H, d, J 7.3), 7.86-7.72 (3H, m), 7.65-7.43 (5H, m), 6.21 (1H, m), 3.52 (4H, br s), 1.75 (3H, d, J 7.1), 1.52-1.41 (2H, m) and 1.32-1.08 (4H, m).

### (R)—N-(1-(Naphthalen-2-yl)ethyl)-2-(piperidin-1-yl)quinazolin-4-amine hydrochloride (Compound 100)

LCMS RT=4.72 min; MH⁺=383.2; ¹H NMR (DMSO): 11.85 (1H, s, HCl), 9.74 (1H, d, J 4.6), 8.54 (1H, d, J 8.1), 7.96-7.73 (6H, m), 7.64 (1H, d, J 8.5), 7.54-7.42 (3H, m), 5.60 (1H, m), 3.78 (4H, br s), 1.73 (3H, d, J 7.1) and 1.65-1.35 (6, H, m).

### N-(3,4-Difluorobenzyl)-2-(piperidin-1-yl)quinazolin-4-amine hydrochloride (Compound 116)

LCMS RT=4.61 min, MH⁺ 355.2; ¹H NMR (DMSO): 11.80 (1H, br s), 10.16 (1H, s), 8.33 (1H, d, J 8.1), 7.85-7.76 (2H, m), 7.56-7.34 (3H, m), 7.30-7.22 (1H, m), 4.75 (2H, d, J 5.5), 3.87-3.76 (4H, m) and 1.70-1.49 (6H, br m).

### N-(2,4-Difluorobenzyl)-2-(piperidin-1-yl)quinazolin-4-amine hydrochloride (Compound 115)

LCMS RT=4.65 min, MH⁺ 355.3; ¹H NMR (DMSO): 11.97 (1H, br s), 10.17-10.09 (1H, br m), 8.36 (1H, d, J 8.3), 7.87-7.77 (2H, m), 7.58-7.40 (2H, m), 7.32-7.22 (1H, m), 7.12-7.02 (1H, td, J 8.5 and 2.5), 4.77 (2H, d, J 5.5), 3.88-3.77 (4H, m) and 1.71-1.49 (6H, br m).

### 2-(Piperidin-1-yl)-N-(4-(trifluoromethyl)phenethyl)quinazolin-4-amine (Compound 105)

LCMS RT=4.73 min, MH⁺ 401.3; ¹H NMR (DMSO): 11.70 (1H, br s), 9.55 (1H, br s), 8.19 (1H, d, J 8.3), 7.85-7.61 (4H, br m), 7.52-7.38 (3H, br s), 3.91-3.78 (6H, br m), 3.08 (2H, t, J 6.9) and 1.75-1.58 (6H, br m).

### N-(2-Methylbenzyl)-2-(piperidin-1-yl)quinazolin-4-amine hydrochloride (Compound 104)

LCMS RT=4.66 min, MH⁺ 333.3; ¹H NMR (DMSO): 11.79 (1H, br s), 9.87 (1H, br s), 8.35 (1H, d, J 8.2), 7.86-7.72 (2H, br m), 7.44 (1H, t, J 7.2), 7.32-7.25 (1H, br m), 7.24-7.11 (3H, br m), 4.78 (2H, d, J 5.5), 3.85-3.74 (4H, br m), 2.36 (3H, s—obscured by solvent signal) and 1.71-1.49 (6H, br m).

### N-(4-Methylphenethyl)-2-(piperidin-1-yl)quinazolin-4-amine hydrochloride (Compound 87)

LCMS RT=4.65 min, MH⁺ 347.2; ¹H NMR (DMSO): 11.77 (1H, br s), 9.58 (1H, br s), 8.21 (1H, d, J 8.1), 7.83-7.72 (2H, br m), 7.42 (1H, t, J 7.5), 7.16-7.07 (4H, br m), 3.91-3.81 (4H, br m), 3.80-3.69 (2H, br m), 2.93 (2H, t, J 7.3), 2.25 (3H, s) and 1.75-1.59 (6H, br m).

### N-(2,3-Dihydro-1H-inden-2-yl)-2-(piperidin-1-yl)quinazolin-4-amine hydrochloride (Compound 86)

LCMS RT=4.68 min, MH⁺ 345.3; ¹H NMR (DMSO): 12.04 (1H, br s), 9.53 (1H, d, J 6.1), 8.42 (1H, d, J 8.1), 7.88 (1H, d, J 8.3), 7.79 (1H, t, J 7.5), 7.4 (1H, t, J 7.5), 7.31-7.15 (4H, br m), 5.05 (1H, br m), 4.93-3.82 (4H, br m), 3.45-3.35 (2H, br m—obscured by water signal), 3.25-3.11 (2H, br m) and 1.75-1.54 (6H, br m).

### 4-(Isoindolin-2-yl)-2-(piperidin-1-yl)quinazoline hydrochloride (Compound 85)

LCMS RT=4.62 min, MH⁺ 331.3; ¹H NMR (DMSO): 11.92 (1H, br s), 8.48 (1H, d, J 8.3), 7.91-7.81 (2H, br m), 7.55-7.43 (3H, br m), 7.43-7.35 (2H, br m), 5.69-5.50 (2H, br m), 5.32-5.15 (2H, br m), 3.97-3.84 (4H, br m) and 1.76-1.61 (6H, br m).

### N-(4-Methoxyphenethyl)-2-(piperidin-1-yl)quinazolin-4-amine hydrochloride (Compound 84)

LCMS RT=4.63 min, MH⁺ 363.2; ¹H NMR (DMSO): 11.86 (1H, br s), 9.61 (1H, br s), 8.23 (1H, d, J 8.2), 7.80 (2H, d, J 4.0), 7.46-7.38 (1H, br m), 7.16 (2H, d, J 8.7), 6.86 (2H, d, J 8.7), 3.93-3.82 (4H, br m), 3.79-3.68 (5H, br m), 2.91 (2H, t, J 7.2) and 1.76-1.58 (6H, br m).

### N-(4-Chlorophenethyl)-2-(piperidin-1-yl)quinazolin-4-amine hydrochloride (Compound 83)

LCMS RT=4.68 min, MH⁺ 367.2; ¹H NMR (DMSO): 11.86 (1H, br s), 9.50 (1H, br s), 8.17 (1H, d, J 8.0). 7.83-7.74 (1H, br m), 7.76-7.63 (1H, br m), 7.47-7.32 (3H, br m), 7.31-7.24 (2H, d, J 8.6), 3.89-3.73 (6H, br m), 2.98 (2H, t, J 7.2) and 1.74-1.58 (6H, br m).

### N-(3-Methylbenzyl)-2-(piperidin-1-yl)quinazolin-4-amine hydrochloride (Compound 80)

LCMS RT=4.62 min, MH⁺ 333.3; ¹H NMR (DMSO): 11.79 (1H, br s), 10.07 (1H, br s), 8.31 (1H d, J 8.3), 7.85-7.71 (2H, br m), 7.44 (1H, td, J 7.6 and 1.5), 7.26-7.16 (3H, br m), 7.11-7.05 (1H, d, J 6.5), 4.73 (2H, d, J 5.8), 3.87-3.78 (4H, br m), 2.28 (3H, s) and 1.71-1.50 (6H, br

### N-(2-Methoxybenzyl)-2-(piperidin-1-yl)quinazolin-4-amine hydrochloride (Compound 81)

LCMS RT=4.60 min, MH⁺ 349.2; ¹H NMR (DMSO): 12.02 (1H, br s), 10.03 (1H, br s), 8.39 (1H, d, J 8.3), 7.88 (1H, d, J 8.5), 7.80 (1H, td, J 7.8 and 1.2), 7.43 (1H, td, J 7.6 and 1.2), 7.30-7.22 (2H, br m), 7.02 (1H, td, J 8.7 and 0.9), 6.89 (1H, td, J 7.4 and 1.0), 4.74 (2H, d, J 5.6) and 3.87-3.76 (7H, br m), 1.68-1.47 (6H, br m).

### (S)-3-Phenyl-2-(2-(piperidin-1-yl)quinazolin-4-ylamino)propan-1-ol hydrochloride (Compound 143)

LCMS RT=4.32 min, MH⁺ 363.3; ¹H NMR (DMSO): 11.71 (1H, br s), 9.20-9.05 (1H, br m), 8.38 (1H, d, J 7.9), 7.83-7.67 (2H, br m), 7.42 (1H, d, J 7.9), 7.30-7.17 (4H, br m), 7.17-7.08 (1H, br m), 5.03 (1H, br s), 4.70-4.55 (1H, br m), 3.90-3.72 (4H, br m), 3.71-3.55 (4H, br m), 3.08-2.85 (2H, br m) and 1.74-1.49 (6H, br m).

### (R)-2-Phenyl-2-(2-(piperidin-1-yl)quinazolin-4-ylamino)ethanol hydrochloride (Compound 145),

LCMS RT=4.53 min, MH⁺ 349.3 ¹H NMR (DMSO): 11.85 (1H, br s), 9.70 (1H, br s), 8.61 (1H, br s), 7.87-7.69 (2H, br m), 7.51-7.39 (3H, br m), 7.37-7.29 (2H, t, J 7.0), 7.29-7.20 (1H, br m), 5.38-5.18 (2H, br m), 4.02-3.88 (1H, br m), 3.87-3.68 (5H, br m), and 1.70-1.38 (6H, br m).

### N-((5-Chlorothiophen-2-yl)methyl)-2-(piperidin-1-yl)quinazolin-4-amine (Compound 189)

LCMS RT=1.66 min, MH⁺ 358.9; ¹H NMR (DMSO-d6): 12.02 (1H, br s), 10.25 (1H, br s), 8.28 (1H, d, J 8.4), 7.90-7.79 (2H, m), 7.49-7.41 (1H, m), 7.07-7.00 (2H, m), 4.88 (2H, d, J 5.6), 3.99-3.90 (4H, m) and 1.78-1.62 (6H, m).

### N-(Furan-3-ylmethyl)-2-(piperidin-1-yl)quinazolin-4-amine (Compound 190)

LCMS RT=1.58 min, MH⁺ 309.2; ¹H NMR (DMSO-d6): 11.98 (1H, br s), 9.97 (1H, br s), 8.31 (1H, d, J 8.1), 7.90-7.77 (2H, m), 7.72 (1H, s), 7.64 (1H, t, J 1.5), 7.47-7.40 (1H, m), 6.55 (1H, d, J 1.9), 4.64 (2H, d, J 5.6), 3.94-3.87 (4H, m) and 1.74-1.60 (6H, m).

### 2-(Piperidin-1-yl)-N-(thiophen-3-ylmethyl)quinazolin-4-amine (Compound 191)

LCMS RT=1.63 min, MH⁺ 325.2; ¹H NMR (DMSO-d6): 11.99 (1H, s), 10.11 (1H, br s), 8.34 (1H, d, J 8.2), 7.90-7.77 (2H, m), 7.55-7.50 (1H, m), 7.49-7.41 (2H, m), 7.18 (1H, d, J 5.0 and 1.3), 4.79 (2H, d, J 5.7), 3.98-3.85 (4H, m) and 1.72-1.57 (6H, m).

### N-((6-Chloropyridin-3-yl)methyl)-2-(piperidin-1-yl)quinazolin-4-amine hydrochloride (Compound 1′94)

LCMS RT=1.56 min; MH⁺ 354.2; ¹H NMR (DMSO): 11.92 (1H, s), 10.20 (1H, br s), 8.51-8.46 (1H, m), 8.32 (1H, d, J 8.1), 7.90 (1H, dd, J 8.2 and 2.2), 7.80 (2H, m), 7.52-7.40 (2H, m), 4.78 (2H, d, J 5.2), 3.87-3.76 (4H, m) and 1.71-1.48 (6H, m).

### 2-(Piperidin-1-yl)-N-((6-(trifluoromethyl)pyridin-3-yl)methyl)quinazolin-4-amine hydrochloride (Compound 196)

LCMS RT=1.64 min; MH⁺ 388.2; ¹H NMR (DMSO): 11.92 (1H, s), 10.27 (1H, br s), 8.85-8.83 (1H, m), 8.34 (1H, d, J 7.8), 7.93 (1H, dd, J 7.9 and 1.7), 7.88 (1H, d, J 8.0), 7.84-7.78 (2H, m), 7.49-7.42 (1H, m), 4.90 (2H, d, J 5.4), 3.81-3.74 (4H, m) and 1.68-1.44 (6H, m).

### Method 4q

### Compounds of General Formula (I)

### 2-(Piperidin-1-yl)-N-(quinolin-4-ylmethyl)quinazolin-4-amine

4-Chloro-2-(piperidin-1-yl)quinazoline (74 mg, 0.3 mmol) was suspended in MeCN (Solvent S, 2 mL), and treated with 4-aminomethylquinoline hydrochloride (58 mg, 0.3 mmol, 1.0 eq. E). The mixture was heated to 180° C. (Temperature K) for 10 minutes (Time T) under microwave irradiation. After cooling to room temperature, a white precipitate formed which was collected by filtration. The precipitate was then partitioned between EtOac and aq. NaHCO₃. The organic layer was concentrated, and then purified by column chromatography (1:1 EtOAc-petrol to 100% EtOAc to afford the title compound as an off-white solid (35 mg, 32%).

LCMS RT=1.43 min; MH⁺ 370.1; ¹H NMR (CDCl₃): 8.82 (1H, d, J 4.3), 8.19-8.11 (2H, m), 7.78-7.71 (1H, m), 7.63-7.48 (4H, m), 7.41-7.36 (1H, m), 7.08-7.00 (1H, m), 6.07 (1H, br s), 5.27 (2H, d, J 5.3), 3.82-3.72 (4H, m) and 1.66-1.46 (6H, m).

### 6. Methods 12-13

### Boc Deprotection and Piperazine Acylation

### Method 12

### Compounds of General Formula (I) from Other Compounds of General Formula (I)

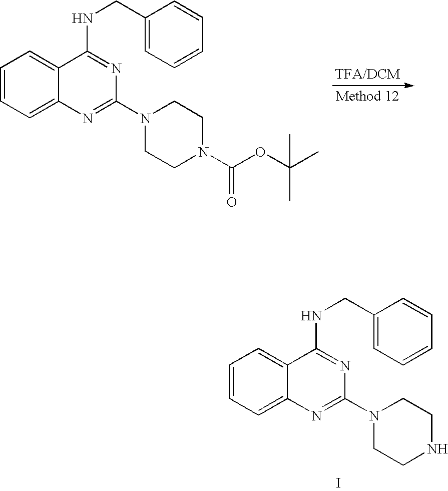

### N-Benzyl-2-(piperazin-1-yl)quinazolin-4-amine (Compound 51)

tert-Butyl 4-(4-(benzylamino)quinazolin-2-yl)piperazine-1-carboxylate (Compound 31; 1.08 g, 2.60 mmol) was dissolved in DCM (20 mL) and treated with TFA (20 mL). The reaction was stirred at RT for 15 min, then concentrated in vacuo. The resulting oil was taken up in water and basified with NaHCO₃. The solid that formed was collected by filtration, washed with water and dried in a vacuum oven. Column chromatography in EtOAc-EtOAc/10% MeOH gave the product as a pale pink solid (410 mg, 49%).

LCMS RT=6.73 min, MH⁺ 320.3; ¹H NMR (DMSO): 8.54 (1H, t, J 5.5, NH), 8.02 (1H, d, J 8.3), 7.49 (1H, ddd, J 8.2, 7.0 and 1.2), 7.40-7.18 (6H, m), 7.05 (1H, ddd, J 8.2, 7.0 and 1.2), 4.68 (2H, d, J 5.8), 3.64 (4H, t, J 4.7) and 2.65 (4H, t, J 5.0).

### Method 13

### Compounds of General Formula (I) in which NR³R⁴ is Piperazinyl Substituted with COR⁶

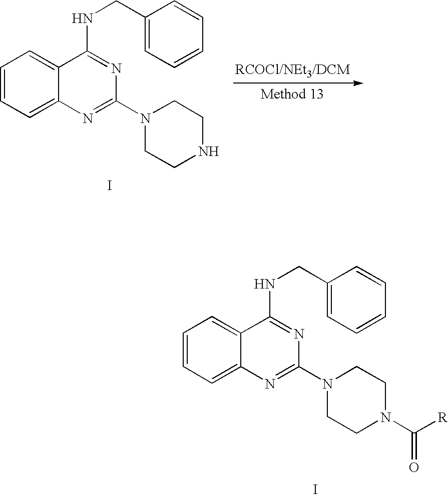

### 1-(4-(4-(Benzylamino)quinazolin-2-yl)piperazin-1-yl)-2-methylpropan-1-one (Compound 56)

To a solution of N-benzyl-2-(piperazin-1-yl)quinazolin-4-amine (Compound 51; 96 mg, 0.30 mmol) in DCM (2 mL) was added triethylamine (125 □L, 0.90 mmol), followed by isobutyroyl chloride (35 □L, 0.33 mmol). The reaction was heated in the microwave at 100° C. for 10 min. The crude reaction was then adsorbed on silica and purified by flash chromatography (ethyl acetate/petrol ether 1:1) to give the product as a white solid (95 mg, 81%).

LCMS RT=5.64 min, MH⁺ 362.2; ¹H NMR (DMSO): 8.64 (1H, t, J 5.9, NH), 8.05 (1H, dd, J 8.2 and 1), 7.52 (1H, ddd, J 8.5, 7.0 and 1.3), 7.41-7.18 (6H, m), 7.09 (1H, ddd, J 8.1, 7.1 and 1.2), 4.70 (2H, d, J 5.7), 3.80-3.64 (4H, m), 3.53-3.42 (4H, m), 2.89 (1H, tt, J 6.7) and 1.00 (6H, d, J 6.6).

The following compounds were prepared in a similar manner, purifying by crystallisation or column chromatography where necessary:

### 1-(4-(4-(Benzylamino)quinazolin-2-yl)piperazin-1-yl)-2,2-dimethylpropan-1-one (Compound 57)

LCMS RT=6.86 min, MH⁺ 404.3; ¹H NMR (DMSO): 8.63 (1H, t, J 5.5, NH), 8.05 (1H, dd, J 8.2 and 0.8), 7.52 (1H, ddd, J 8.4, 6.9 and 1.4), 7.41-7.18 (6H, m), 7.09 (1H, ddd, J 8.0, 7.0 and 1.0), 4.71 (2H, d, J 5.6), 3.75-3.66 (4H, m), 3.58-3.50 (4H, m) and 1.21 (9H, s).

### 1-(4-(4-(Benzylamino)quinazolin-2-yl)piperazin-1-yl)(phenyl)methanone (Compound 58)

LCMS RT=6.52 min, MH⁺ 424.2; ¹H NMR (DMSO): 8.65 (1H, t, J 6.0, NH), 8.05 (1H, dd, J 8.2 and 0.9), 7.55 (1H, ddd, J 8.4, 6.9 and 1.3), 7.49-7.34 (7H, m), 7.33-7.26 (3H, m), 7.24-7.17 (2H, m), 7.10 (1H, ddd, J 8.1, 7.0 and 1.1), 4.69 (2H, d, J 5.9), 3.87-3.68 (4H, m), 3.66-3.52 (2H, m) and 3.40-3.25 (2H, obsc.).

### 5. Methods 8-9

### Quinoline Compounds of General Formula (I)

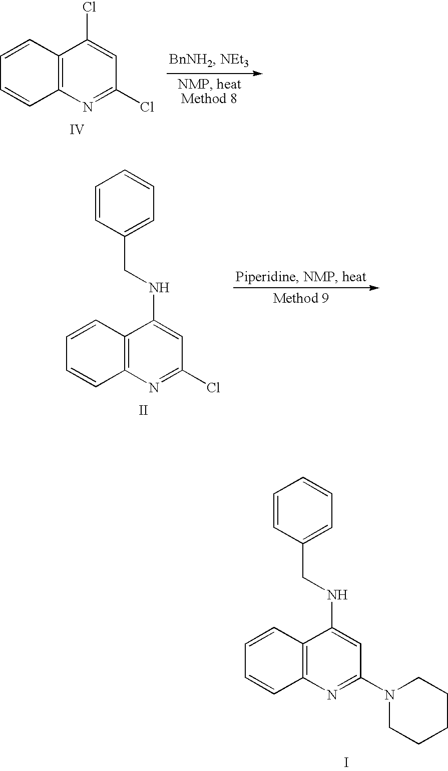

### Method 8

### Compounds of General Formula (II) in which X¹ is CH, R¹ is H and R² is Benzyl

### N-Benzyl-2-chloroquinolin-4-amine

2,4-Dichloroquinoline (300 mg, 1.51 mmol) was dissolved in NMP (5 mL). Triethylamine (1.00 mL, 7.56 mmol) was added, followed by benzylamine (1984, 1.80 mmol). The mixture was heated to 150° C. for 17 h. The mixture was cooled to room temperature, diluted with water (to form a precipitate) and EtOAc. The mixture was washed with brine (3×20 mL), the organic layer dried (MgSO₄), filtered and concentrated. The crude mixture was purified by column chromatography (5% EtOAc-petrol to 20% EtOAc-petrol) to afford the title compound as a yellow solid (84 mg, 22%). This material was carried forward to further reaction.

¹H NMR (DMSO): 7.88 (1H, app. d), 7.69 (1H, t, J 5.7), 7.60-7.53 (2H, m), 7.41-7.21 (6H, m), 7.06 (1H, s) and 4.63 (2H, d, J 5.9).

### Method 9

### Compounds of General Formula (I) in which X¹ is CH, R¹ is H and R² is Benzyl

### N-Benzyl-2-(piperidin-1-yl)quinolin-4-amine (Compound 54)

N-Benzyl-2-chloroquinolin-4-amine (73 mg, 0.29 mmol) was dissolved in NMP (2 mL). Piperidine (86 μL, 0.87 mmol) was added and the mixture heated to 150° C. for 17 h. The brown solution was cooled to room temperature, and diluted with EtOAc and water. The mixture was washed with brine (3×20 mL) and the organic layer dried (MgSO₄), filtered and concentrated. The crude product was purified by column chromatography (10-100% EtOAC-petrol) to afford the title compound as a yellow solid (28 mg, 32%).

LCMS RT=8.47 min, MH⁺ 318.2; ¹H NMR (DMSO): 7.69 (1H, dd, J 8.3 and 0.9), 7.45-7.19 (8H, m), 7.11 (1H, ddd, J 8.1, 6.5 and 1.7), 6.31 (1H, s), 3.02-2.96 (4H, m), 1.79-1.72 (4H, m) and 1.65-1.58 (2H, m).

### 6. Miscellaneous Methods

### Method 15

### Compounds of General Formula (I) in which R³ is H and R⁴ is COR⁵

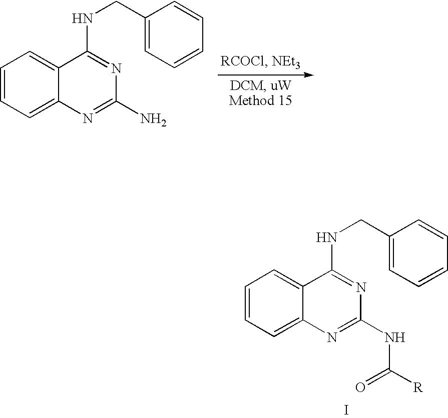

### N-(4-(Benzylamino)quinazolin-2-yl)benzamide (Compound 73)

N⁴-Benzylquinazoline-2,4-diamine (100 mg, 0.4 mmol) was suspended in DCM (3 mL). NEt₃ (2 eq.) and benzoyl chloride (51 μL, 1.1 eq.) were added and the mixture heated to 100° C. for 10 min under microwave irradiation. The mixture was cooled to ambient temperature and absorbed onto silica. Column chromatography (2:1 petrol:EtOAc) afforded the crude product, which was triturated with petrol to give the product as an off-white powder (60 mg, 42%).

LCMS RT=4.97 min, MH⁺ 355.2; ¹H NMR (DMSO): 10.44 (1H, s), 8.86-8.77 (1H, br m), 8.23 (1H, d, J 8.1), 7.92 (2H, d, J 7.5), 7.72 (1H, t, J 7.6), 7.61-7.52 (2H, br m), 7.52-7.36 (5H, br m), 7.31 (2H, t, J 7.4), 7.27-7.19 (1H, br m) and 4.75 (2H, d, J 5.7).

### Method 20

### Curtius Rearrangement

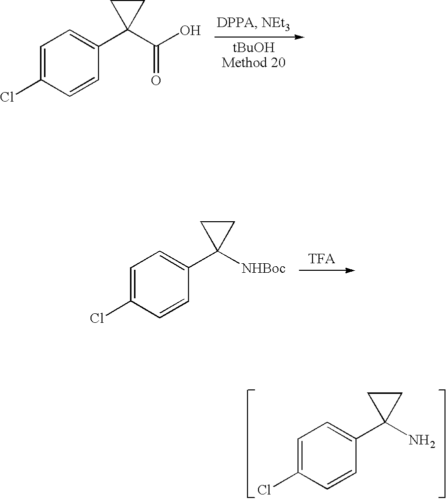

### 1-(4-Chlorophenyl)cyclopropanamine

1-(4-Chlorophenyl)cyclopropanecarboxylic acid (590 mg, 3.0 mmol) was suspended in ^{t}BuOH (5 mL). This suspension was treated with NEt₃ (834 μL, 6.0 mmol, 2 eq.) and diphenyl phosphoryl azide (DPPA, 647 μL, 3.0 mmol, 1 eq.) and stirred at 80° C. for 18 h. The solvent was removed in vacuo and the residue taken up in EtOAc and washed with aq. NaHCO₃, 1M NaOH and brine. The EtOAc layer was concentrated. This residue was extracted with Et₂O and then the Et₂O extract absorbed onto silica and purified by column chromatography (1:1 EtOAc-petrol) to give the Boc-amine as an off-white solid. This material was dissolved in DCM (5 mL) and treated with TFA (5 mL). The mixture was stirred at room temperature for 2 h, then concentrated in vacuo. The residue was taken up in aq. NaHCO₃ solution and extracted with EtOAc. The organic extracts were washed with 1M NaOH and brine, then dried (MgSO₄), filtered and concentrated. The crude amine was used without further purification or analysis.

Table 1 shows for each of the compounds the compound number, the method by which it was obtained, the solvent (S) and temperature (K) used for the reaction; the number of molar equivalents of reagent (E) and base (EB) if used as well as the time (T) for which microwave irradiation was applied.

TABLE 1

| Compound | | | | | | |
|---|---|---|---|---|---|---|
| Number | Method | S | E | EB | Time T | Temp K |
| 1 | 2a | IMS | 3 | n/a | 5 | 150 |
| 2 | Commercial | n/a | n/a | n/a | n/a | n/a |
| 1 | 2a | IMS | 3 | n/a | 5 | 150 |
| 2 | Commercial | n/a | n/a | n/a | n/a | n/a |
| 3 | 2b | IMS | 10 + 10 + 13 | 2 | 3 × 15 + 20 + 15 | 150-175 |
| 4 | Commercial | n/a | n/a | n/a | n/a | n/a |
| 5 | Commercial | n/a | n/a | n/a | n/a | n/a |
| 6 | Commercial | n/a | n/a | n/a | n/a | n/a |
| 7 | Commercial | n/a | n/a | n/a | n/a | n/a |
| 8 | Commercial | n/a | n/a | n/a | n/a | n/a |
| 9 | Commercial | n/a | n/a | n/a | n/a | n/a |
| 10 | Commercial | n/a | n/a | n/a | n/a | n/a |
| 11 | Commercial | n/a | n/a | n/a | n/a | n/a |
| 12 | Commercial | n/a | n/a | n/a | n/a | n/a |
| 13 | Commercial | n/a | n/a | n/a | n/a | n/a |
| 14 | Commercial | n/a | n/a | n/a | n/a | n/a |
| 15 | Commercial | n/a | n/a | n/a | n/a | n/a |
| 16 | Commercial | n/a | n/a | n/a | n/a | n/a |
| 17 | 2a | IMS | 3 | n/a | 5 | 150 |
| 18 | Commercial | n/a | n/a | n/a | n/a | n/a |
| 19 | Commercial | n/a | n/a | n/a | n/a | n/a |
| 20 | Commercial | n/a | n/a | n/a | n/a | n/a |
| 21 | Commercial | n/a | n/a | n/a | n/a | n/a |
| 22 | 2b | IMS | 3 | 2 | 10 | 150 |
| 23 | Commercial | n/a | n/a | n/a | n/a | n/a |
| 24 | 2b | NMP | 1.2 | 3 | 10 | 210 |
| 25 | 2b | IMS | 3 | 2 | 3 × 10 | 150 |
| 26 | 2b | IMS | 1.05 | 2 | 2 × 15 | 150 |
| 27 | 2b | IMS | 1.05 | 2 | 15 | 150 |
| 28 | 2b | IMS | 1.1 | 2 | 2 × 20 | 150 |
| 29 | 2b | IMS | 1.1 | 2 | 2 × 15 | 150 |
| 30 | 2b | IMS | 1.1 | 2 | 2 × 15 | 150 |
| 31 | 2b | IMS | 1.05 | 2 | 2 × 15 | 150 |
| 32 | 4a | IPA | 1.05 + 0.5 | 2 | 20 + 10 | 180 |
| 33 | 4a | IPA | 1.05 + 0.5 | 2 | 20 + 10 | 180 |
| 34 | 4a | IMS | 1.05 | 2 | 4 × 15 | 150 |
| 35 | 4b | Pyr | 1.05 | n/a | 10 | 200 |
| 36 | 4b | Pyr | 1.05 | n/a | 10 | 200 |
| 37 | 4a | IPA | 2 × 1.05 | 2 | 2 + 1 × 15 | 160-170 |
| 38 | 4a | IPA | 1.05 + 0.5 | 2 | 20 + 10 | 180 |
| 39 | 4a | IPA | 1.05 + 0.5 | 2 | 20 + 10 | 180 |
| 40 | 4a | IPA | 1.05 | 2 | 20 | 180 |
| 41 | 4a | IPA | 1.05 | 2 | 20 | 180 |
| 42 | 4a | IPA | 1.05 | 2 | 20 | 180 |
| 43 | 4a | IPA | 1.1 | 2 | 15 | 180 |
| 44 | 4a | IPA | 1.1 | 2 | 15 | 180 |
| 45 | 4a | IPA | 1.1 | 2 | 15 | 180 |
| 46 | 4d | IPA | 1.05 | n/a | 15 | 180 |
| 47 | 4d | IPA | 1.05 | n/a | 15 | 180 |
| 48 | 4d | IPA | 1.05 | n/a | 15 | 180 |
| 49 | 4a | IPA | 1.05 | 2 | 20 | 180 |
| 50 | 2a | IMS | 3 | n/a | 10 | 150 |
| 51 | 12 | n/a | n/a | n/a | n/a | n/a |
| 52 | 2a | MeCN | 1.1 | n/a | 10 | 200 |
| 53 | 2a | MeCN | 2 | n/a | 10 | 200 |
| 54 | 9 | n/a | n/a | n/a | n/a | n/a |
| 55 | 2a | MeCN | 1.1 | n/a | 10 | 200 |
| 56 | 13 | n/a | n/a | n/a | n/a | n/a |
| 57 | 13 | n/a | n/a | n/a | n/a | n/a |
| 58 | 13 | n/a | n/a | n/a | n/a | n/a |
| 59 | 2a | IMS | 3 | n/a | 10 | 150 |
| 60 | 2a | IMS | 3 | n/a | 10 | 150 |
| 61 | 2a | IMS | 3 | n/a | 10 | 150 |
| 62 | 2a | MeCN | 3 | n/a | 5 | 180 |
| 63 | 4a | IPA | 1.05 + 0.5 | 2 | 15 + 15 | 180 |
| 64 | 4a | IPA | 1.05 + 1 | 2 | 15 + 15 | 180 |
| 65 | 4a | IPA | 1.05 + 1 | 2 | 15 + 15 | 180 |
| 66 | 4a | MeCN | 1.05 + 0.5 | 2 | 15 + 2 × 15 | 150-180 |
| 67 | 2a | MeCN | 2 | n/a | 15 | 180 |
| 68 | 4a | IPA | 1.5 | 2 | 15 | 180 |
| 69 | 4a | IPA | 1.5 | 2 | 15 | 180 |
| 70 | 2a | MeCN | 3 | n/a | 15 | 150 |
| 71 | 2a | MeCN | 3 | n/a | 10 + 15 | 180 |
| 72 | 4e | IPA | 1.05 | 3 | 10 | 150 |
| 73 | 15 | n/a | n/a | n/a | n/a | n/a |
| 74 | 2a | MeCN | 3 | n/a | 15 | 180 |
| 75 | 2a | MeCN | 3 | n/a | 15 | 180 |
| 76 | 2b | MeCN | 1.05 + 1 | 2 | 15 + 2 × 15 | 180 |
| 77 | 2a | MeCN | 3 | n/a | 15 | 180 |
| 78 | 2a | MeCN | 3 | n/a | 15 | 180 |
| 79 | 2b | MeCN | 1.05 + 1 | n/a | 15 + 2 × 15 | 180 |
| 80 | 4f | MeCN | 1.05 | n/a | 2 × 10 | 200 |
| 81 | 4f | MeCN | 1.05 | n/a | 2 × 10 | 200 |
| 82 | 4f | MeCN | 1.05 | n/a | 10 | 200 |
| 83 | 4f | MeCN | 1.05 | n/a | 2 × 10 | 200 |
| 84 | 4f | MeCN | 1.05 | n/a | 10 | 200 |
| 85 | 4f | MeCN | 1.05 | n/a | 10 | 200 |
| 86 | 4f | MeCN | 1.05 | n/a | 10 | 200 |
| 87 | 4f | MeCN | 1.05 | n/a | 2 × 10 | 200 |
| 88 | 4d | MeCN | 1.05 | n/a | 10 | 200 |
| 89 | 4d | MeCN | 1.05 | n/a | 10 | 200 |
| 90 | 4d | MeCN | 1.05 | n/a | 10 | 200 |
| 91 | 2a | MeCN | 2 | n/a | 10 | 200 |
| 92 | 4d | MeCN | 1.1 | n/a | 10 | 200 |
| 93 | 4d | MeCN | 1.1 | n/a | 10 | 200 |
| 94 | 4d | MeCN | 1.1 | n/a | 10 | 200 |
| 95 | 4d | MeCN | 1.1 | n/a | 10 | 200 |
| 96 | 4f | MeCN | 1.05 | n/a | 10 | 200 |
| 97 | 4d | MeCN | 1.05 | n/a | 10 | 200 |
| 98 | 4f | MeCN | 1.05 | n/a | 10 | 200 |
| 99 | 4f | MeCN | 1.05 | n/a | 10 | 200 |
| 100 | 4f | MeCN | 1.05 | n/a | 10 | 200 |
| 101 | 4d | MeCN | 1.05 | n/a | 10 | 200 |
| 102 | 2a | MeCN | 2 | n/a | 10 | 200 |
| 103 | 2a | MeCN | 2 | n/a | 10 | 200 |
| 104 | 4f | MeCN | 1.05 | n/a | 10 | 200 |
| 105 | 4f | MeCN | 1.05 | n/a | 10 | 200 |
| 106 | 2d | MeCN | 1.05 | 2 | 15 | 180 |
| 107 | 2a | MeCN | 3 | n/a | 10 | 180 |
| 108 | 2a | MeCN | 3 | n/a | 10 | 180 |
| 109 | 2c | MeCN | 1.05 | n/a | 3 × 10 | 180 |
| 110 | 2c | MeCN | 1.05 | n/a | 3 × 10 | 180 |
| 111 | 2c | MeCN | 1.05 | n/a | 3 × 10 | 180 |
| 112 | 2c | MeCN | 1.05 | n/a | 3 × 10 | 180 |
| 113 | 2d | MeCN | 1.05 | n/a | 10 | 180 |
| 114 | 2d | MeCN | 1.05 | n/a | 15 | 180 |
| 115 | 4f | MeCN | 1.05 | n/a | 10 | 200 |
| 116 | 4f | MeCN | 1.05 | n/a | 10 | 200 |
| 117 | 4d | MeCN | 1.05 | n/a | 10 | 200 |
| 118 | 2c | MeCN | 1.05 | n/a | 10 | 200 |
| 119 | 2c | MeCN | 0.95 | n/a | 10 | 200 |
| 120 | 2a | MeCN | 1.2 | n/a | 10 | 200 |
| 121 | 2a | MeCN | 1.2 | n/a | 10 | 200 |
| 122 | 2a | MeCN | 1.2 | n/a | 10 | 200 |
| 123 | 2a | MeCN | 1.2 | n/a | 10 | 200 |
| 124 | 2a | MeCN | 1.2 | n/a | 5 | 190 |
| 125 | 2a | MeCN | 1.2 | n/a | 5 | 190 |
| 126 | 2c | MeCN | 1.05 | n/a | 10 | 200 |
| 127 | 2c | MeCN | 1.05 | n/a | 10 | 200 |
| 128 | 2c | MeCN | 1.05 | n/a | 10 | 200 |
| 129 | 2c | MeCN | 1.05 | n/a | 10 | 200 |
| 130 | 2a | MeCN | 1.05 | n/a | 10 | 200 |
| 131 | 2a | MeCN | 1.05 | n/a | 10 | 200 |
| 132 | 2c | MeCN | 1.05 | n/a | 10 | 200 |
| 133 | 2c | MeCN | 1 | n/a | 10 | 190 |
| 134 | 2c | MeCN | 1 | n/a | 10 | 190 |
| 135 | 2c | MeCN | 1 | n/a | 10 | 190 |
| 136 | 2c | MeCN | 1 | n/a | 10 | 190 |
| 137 | 2f | MeCN | 2 | n/a | 5 | 190 |
| 138 | 2f | MeCN | 1.2 | n/a | 5 | 190 |
| 139 | 2a | MeCN | 1 | n/a | 10 | 200 |
| 140 | 2c | MeCN | 1 | n/a | 10 | 200 |
| 141 | 2c | MeCN | 1 | n/a | 5 | 190 |
| 142 | 2c | MeCN | 1 | n/a | 5 | 190 |
| 143 | 4f | MeCN | 1 | n/a | 10 | 200 |
| 144 | 4d | MeCN | 1 | n/a | 5 | 190 |
| 145 | 4f | MeCN | 1.05 | n/a | 5 | 190 |
| 146 | 2a | MeCN | 1.1 | n/a | 3 × 10 | 180 |
| 147 | 2a | MeCN | 1.1 | n/a | 3 × 10 | 180 |
| 148 | 2a | MeCN | 1.1 | n/a | 2 × 10 | 180 |
| 149 | 2a | MeCN | 1.1 | n/a | 2 × 10 | 180 |
| 150 | 2c | MeCN | 1.05 | n/a | 10 | 200 |
| 151 | 2c | MeCN | 1.05 | n/a | 10 | 200 |
| 152 | 4g | MeCN | 2 | n/a | 5 | 190 |
| 153 | 4g | MeCN | 2 | n/a | 5 | 190 |
| 154 | 4g | MeCN | 2 | n/a | 5 | 190 |
| 155 | 4g | MeCN | 2 | n/a | 5 | 190 |
| 156 | 4g | MeCN | 2 | n/a | 5 | 190 |
| 157 | 4g | MeCN | 2 | n/a | 5 | 190 |
| 158 | 2c | MeCN | 1 | n/a | 10 | 200 |
| 159 | 2c | MeCN | 1 | n/a | 10 | 200 |
| 160 | 2c | MeCN | 1 | n/a | 10 | 200 |
| 161 | 2c | MeCN | 1 | n/a | 10 | 200 |
| 162 | 2f | MeCN | 2 | n/a | 5 | 190 |
| 163 | 2a | MeCN | 2 | n/a | 5 | 190 |
| 164 | 2f | MeCN | 2 | n/a | 5 | 190 |
| 165 | 2f | MeCN | 2 | n/a | 7 | 180 |
| 166 | 2a | MeCN | 2 | n/a | 5 + 20 + 30 | 190 |
| 167 | 2a | MeCN | 1 | n/a | 10 | 200 |
| 168 | 2c | MeCN | 0.95 | n/a | 10 | 200 |
| 169 | 2f | MeCN | 1.2 | n/a | 5 | 180 |
| 170 | 2f | MeCN | 1.2 | n/a | 10 | 180 |
| 171 | 2f | MeCN | 1.2 | n/a | 10 | 180 |
| 172 | 2f | MeCN | 1.2 | n/a | 10 | 180 |
| 173 | 2c | MeCN | 1.2 | n/a | 10 | 180 |
| 174 | 2c | MeCN | 1.2 | n/a | 10 | 180 |
| 175 | 4d | MeCN | 1 | n/a | 5 | 150 |
| 176 | 4d | MeCN | 1 | n/a | 5 + 10 | 150-160 |
| 177 | 2f | MeCN | 1 | n/a | 10 | 180 |
| 178 | 2f | MeCN | 1 | n/a | 10 | 180 |
| 179 | 2c | MeCN | 1.1 | n/a | 10 | 180 |
| 180 | 2f | MeCN | 1 | n/a | 10 | 150 |
| 181 | 2e | MeCN | 1 | 2 | 10 | 160 |
| 182 | 2f | MeCN | 1.2 | n/a | 10 | 180 |
| 183 | 2f | MeCN | 1.2 | n/a | 10 | 180 |
| 184 | 2a | MeCN | 5 + 5 | n/a | 2 × 10 + 10 | 150 |
| 185 | commercial | n/a | n/a | n/a | n/a | n/a |
| 186 | 2c | MeCN | 1.1 | n/a | 5 | 180 |
| 187 | 2c | MeCN | 1.1 | n/a | 5 | 180 |
| 188 | 2e | MeCN | 1.1 | 2 | 10 | 160 |
| 189 | 4f | MeCN | 1.1 | n/a | 5 | 180 |
| 190 | 4f | MeCN | 1.1 | n/a | 5 | 180 |
| 191 | 4f | MeCN | 1.1 | n/a | 5 | 180 |
| 192 | 2e | MeCN | 1.1 | 2 | 10 | 160 |
| 193 | 4f | MeCN | 1.1 | n/a | 10 | 180 |
| 194 | 4f | MeCN | 1 | n/a | 10 | 180 |
| 195 | 4g | MeCN | 1 | n/a | 10 | 180 |
| 196 | 4f | MeCN | 1 | n/a | 10 | 180 |
| 197 | 2c | MeCN | 1.05 | n/a | 10 | 180 |
| 198 | 4g | MeCN | 1 | n/a | 10 | 180 |
| 199 | 4g | MeCN | 1 | n/a | 10 | 180 |
| 200 | 4g | MeCN | 1 | n/a | 10 | 150 |
| 201 | 4g | MeCN | 1 | n/a | 10 | 180 |
| 202 | 4f | MeCN | 1 | n/a | 10 | 180 |
| 203 | 4g | MeCN | 1 | n/a | 10 | 180 |
| 204 | 4g | MeCN | 1 | n/a | 10 | 180 |
| 205 | 4f | MeCN | 1 | n/a | 10 | 180 |
| 206 | 4f | MeCN | 1 | n/a | 10 | 160 |
| 207 | 4g | MeCN | 1 | n/a | 10 | 160 |
| 208 | 4g | MeCN | 1 | n/a | 10 | 160 |
| 209 | 4f | MeCN | 1 + 1 | n/a | 2 × 10 | 160 |

### EXAMPLE 2

### Alamar Blue Susceptibility Test (MABA)

The assay was carried out according to the method described by Collins et al (Antimicrobial Agents and Chemotherapy (1997) 1004-1009) using the H37Rv strain of M. tuberculosis.

Antimicrobial susceptibility testing was performed in black, clear bottomed, 96 well microplates in order to minimize background fluorescence. Outer perimeter wells were filled with sterile water to prevent dehydration in experimental wells. Initial drug dilutions were prepared in either dimethylsulfoxide or distilled deionized water, and subsequent two-fold dilutions were performed in 0.1 ml of 7H9GC (no Tween 80) in the microplates. BACTEC 12B-passaged inocula were initially diluted 1:2 in 7H9GC, and 0.1 ml was added to the wells. Subsequent determination of the bacterial titers yielded 1×10⁶ CFU/ml in plate wells of H₃₇Rv. Frozen inocula were initially diluted 1:20 in BACTEC 12B medium followed by 1:50 dilution in 7H9GC. Addition of 1/10 ml to wells resulted in final bacterial titers of 2.0×10⁶ CFU/ml of H₃₇Rv. Wells containing drug only were used to detect autofluorescence of compounds. Additional control wells consisted of bacteria only (B) and medium only (M). Plates were incubated at 37° C. Starting at day 4 of incubation, 20 μl of 10× alamar blue solution and 12.5 μl of 20% Tween 80 were added to one B well and one M well, and plates were reincubated at 37° C. Wells were observed at 12 and 24 hrs for colour change from blue to pink and for a reading of ≧50,000 fluorescence units (FU). Fluorescence was measured in a Cytofluor II microplate fluorometer with excitation at 530 nm and emission at 590 nm. If the B wells became pink by 24 hrs, reagent was added to the entire plate. If the wells remained blue or ≦50,000 FU was measured, additional M and B wells were tested daily until a colour change occurred, at which time reagents were added to all remaining wells. Plates were then incubated at 37° C. and results were recorded, at 24 hrs post reagent addition. Fluorometric MICs were determined by a background subtraction on all wells with a mean of triplicate M wells. Percent inhibition was performed on all wells with a mean of triplicate M wells. Percent inhibition was defined as 1−(test well FU/mean FU of triplicate B wells)×100. The lowest drug concentration effecting an inhibition of ≧90% was considered the MIC.

### EXAMPLE 3

### Low Oxygen Recovery Assay for Screening Compounds Against Non-Replicating M. tuberculosis

The experiment was conducted as described by Cho et al (Antimicrobial Agents and Chemotherapy, (2007), 1380-1385) in order to determine whether the test compounds had activity against the non replicating phase of M. tuberculosis. The M. tuberculosis used in this experiment is H37Rv with a plasmid with an acetimidase promoter driving a luciferase gene. This strain is maintained as a standard strain and is readily available.

Cultures were thawed, diluted in Middlebrook 7H12 broth (Middlebrook 7H9 broth containing 1 mg/ml Casitone, 5.6 μg/ml palmitic acid, 5 mg/ml bovine serum albumin and 4 μg/ml filter-sterilized catalase), and sonicated for 15 s. The cultures were diluted to obtain an A₅₇₀ of 0.03 to 0.05 and 3000 to 7000 RLUs per 100 μL. This corresponds to 5×10⁵ to approx 2×10⁶ CFU/ml. Twofold serial dilutions of the test compounds were prepared in a volume of 100 μL in black 96-well microtiter plates, and 100 μL of the cell suspension was added. For LORA, the microplate cultures were placed under anaerobic conditions (oxygen concentration less than 0.16%) by using an Anoxomat™ model WS-8080 (MART Microbiology) and three cycles of evacuation and filling with a mixture of 10% H₂, 5% CO₂, and the balance of N₂. An anaerobic indicator strip was placed inside the chamber to visually confirm the removal of oxygen. The plates were incubated at 37° C. for 10 days and then transferred to an ambient gaseous condition (5% CO₂ enriched air) incubator for a 28 hour “recovery”. The numbers of CFU (determined by subculture onto Middlebrook 7H11 agar) during the 10-day incubation did not increase and remained essentially unchanged. On day 11 (after the 28-h aerobic recovery), 100 μL culture was transferred to white 96-well microtiter plates for determination of luminescence.

Luminescence was measured in a Victor multilabel reader (Perkin-Elmer Life Sciences), using a reading time of 1 s. The MIC was defined as the lowest test compound concentration effecting growth inhibition of ≧90% relative to the growth of the controls. The MICs were numerically extrapolated from transformed inhibition-concentration plots so that the MICs were independent of the discrete twofold concentrations of the drug dilutions tested.

The results are given in Table 2 below.

TABLE 2

| Compound | | |
|---|---|---|
| Number | MABA | LORA |
| 1 | − | n.d. |
| (Comparative) | | |
| 2 | − | n.d. |
| (Comparative) | | |
| 3 | ++ | n.d. |
| (Comparative) | | |
| 4 | ++++ | +++ |
| 5 | ++ | ++ |
| 6 | ++ | ++ |
| 7 | ++ | n.d. |
| 8 | ++ | +++ |
| 9 | ++ | ++ |
| 10 | ++ | +++ |
| 11 | ++ | n.d. |
| 12 | +++ | +++ |
| 13 | ++++ | +++ |
| 14 | +++ | +++ |
| 15 | ++ | − |
| 16 | +++ | +++ |
| 17 | ++ | − |
| 18 | +++ | +++ |
| 19 | +++ | +++ |
| 20 | ++ | − |
| 21 | +++ | +++ |
| 22 | ++++ | +++ |
| 23 | +++ | n.d. |
| 24 | +++ | +++ |
| 25 | +++ | n.d. |
| 26 | ++ | n.d. |
| 27 | ++ | n.d. |
| 28 | +++ | n.d. |
| 29 | +++ | n.d. |
| 30 | ++++ | ++++ |
| 31 | +++ | n.d. |
| 32 | +++ | n.d. |
| 33 | ++++ | +++ |
| 34 | ++++ | +++ |
| 35 | ++++ | ++++ |
| 36 | ++++ | +++ |
| 37 | ++++ | ++++ |
| 38 | ++++ | ++++ |
| 39 | ++++ | +++ |
| 40 | ++++ | +++ |
| 41 | ++++ | +++ |
| 42 | ++++ | +++ |
| 43 | ++++ | +++ |
| 44 | ++++ | +++ |
| 45 | ++++ | +++ |
| 46 | ++ | n.d. |
| 47 | ++ | n.d. |
| 48 | ++++ | +++ |
| 49 | ++++ | +++ |
| 50 | +++ | n.d. |
| 51 | ++ | n.d. |
| 52 | +++ | n.d. |
| 53 | ++++ | +++ |
| 54 | ++++ | ++++ |
| 55 | +++ | n.d. |
| 56 | ++ | n.d. |
| 57 | +++ | n.d. |
| 58 | +++ | n.d. |
| 59 | +++ | ++++ |
| 60 | +++ | ++++ |
| 61 | +++ | n.d. |
| 62 | ++++ | +++ |
| 63 | ++++ | +++ |
| 64 | ++++ | +++ |
| 65 | ++++ | +++ |
| 66 | ++++ | +++ |
| 67 | +++ | n.d. |
| 68 | ++++ | ++++ |
| 69 | ++++ | +++ |
| 70 | +++ | n.d. |
| 71 | +++ | n.d. |
| 72 | ++ | n.d. |
| 73 | ++ | n.d. |
| 74 | +++ | n.d. |
| 75 | +++ | n.d. |
| 76 | ++++ | +++ |
| 77 | +++ | n.d. |
| 78 | ++++ | +++ |
| 79 | ++++ | +++ |
| 80 | ++++ | +++ |
| 81 | +++ | n.d. |
| 82 | +++ | n.d. |
| 83 | ++++ | ++++ |
| 84 | ++++ | +++ |
| 85 | +++ | n.d. |
| 86 | +++ | n.d. |
| 87 | +++ | n.d. |
| 88 | ++ | n.d. |
| 89 | +++ | n.d. |
| 90 | +++ | n.d. |
| 91 | +++ | n.d. |
| 92 | ++++ | ++ |
| 93 | ++++ | ++ |
| 94 | +++ | n.d. |
| 95 | ++++ | n.d. |
| 96 | ++++ | +++ |
| 97 | +++ | n.d. |
| 98 | ++++ | +++ |
| 99 | ++++ | +++ |
| 100 | ++++ | +++ |
| 101 | +++ | n.d. |
| 102 | +++ | n.d. |
| 103 | +++ | n.d. |
| 104 | +++ | n.d. |
| 105 | ++++ | ++++ |
| 106 | ++++ | ++++ |
| 107 | +++ | n.d. |
| 108 | +++ | n.d. |
| 109 | +++ | n.d. |
| 110 | +++ | n.d. |
| 111 | +++ | n.d. |
| 112 | ++++ | ++++ |
| 113 | +++ | n.d. |
| 114 | ++++ | ++++ |
| 115 | ++++ | +++ |
| 116 | ++++ | +++ |
| 117 | ++++ | ++++ |
| 118 | +++ | n.d. |
| 119 | +++ | n.d. |
| 120 | ++++ | +++ |
| 121 | +++ | n.d. |
| 122 | +++ | n.d. |
| 123 | +++ | n.d. |
| 124 | ++++ | +++ |
| 125 | ++ | n.d. |
| 126 | +++ | n.d. |
| 127 | ++++ | ++++ |
| 128 | ++++ | ++++ |
| 129 | +++ | n.d. |
| 130 | ++++ | − |
| 131 | ++++ | ++++ |
| 132 | ++++ | ++++ |
| 133 | ++++ | +++ |
| 134 | ++++ | +++ |
| 135 | ++++ | +++ |
| 136 | ++++ | +++ |
| 137 | +++ | n.d. |
| 138 | ++++ | n.d. |
| 139 | ++++ | n.d. |
| 140 | ++++ | n.d. |
| 141 | +++ | n.d. |
| 142 | +++ | n.d. |
| 143 | +++ | n.d. |
| 144 | +++ | n.d. |
| 145 | ++ | n.d. |
| 146 | +++ | n.d. |
| 147 | ++++ | n.d. |
| 148 | +++ | n.d. |
| 149 | +++ | n.d. |
| 150 | +++ | n.d. |
| 151 | +++ | n.d. |
| 152 | +++ | n.d. |
| 153 | ++ | n.d. |
| 154 | ++++ | n.d. |
| 155 | ++++ | n.d. |
| 156 | +++ | n.d. |
| 157 | ++++ | n.d. |
| 158 | +++ | n.d. |
| 159 | ++++ | n.d. |
| 160 | ++++ | n.d. |
| 161 | +++ | n.d. |
| 162 | ++++ | n.d. |
| 163 | +++ | n.d. |
| 164 | +++ | n.d. |
| 165 | +++ | n.d. |
| 166 | +++ | n.d. |
| 167 | ++++ | n.d. |
| 168 | +++ | n.d. |
| 169 | +++ | n.d. |
| 170 | +++ | n.d. |
| 171 | +++ | n.d. |
| 172 | ++++ | n.d. |
| 173 | +++ | n.d. |
| 174 | +++ | n.d. |
| 175 | +++ | n.d. |
| 176 | ++ | n.d. |
| 177 | ++ | n.d. |
| 178 | ++ | n.d. |
| 179 | ++++ | n.d. |
| 180 | ++ | n.d. |
| 181 | +++ | n.d. |
| 182 | +++ | n.d. |
| 183 | +++ | n.d. |
| 184 | ++ | n.d. |
| 185 | ++ | n.d. |
| 186 | +++ | n.d. |
| 187 | +++ | n.d. |
| 188 | +++ | n.d. |
| 189 | ++++ | n.d. |
| 190 | +++ | n.d. |
| 191 | +++ | n.d. |
| 192 | +++ | n.d. |
| 193 | +++ | n.d. |
| 194 | +++ | n.d. |
| 195 | +++ | n.d. |
| 196 | +++ | n.d. |
| 197 | +++ | n.d. |
| 198 | ++ | n.d. |
| 199 | +++ | n.d. |
| 200 | ++ | n.d. |
| 201 | +++ | n.d. |
| 202 | ++ | n.d. |
| 203 | +++ | n.d. |
| 204 | ++ | n.d. |
| 205 | ++ | n.d. |
| 206 | ++ | n.d. |
| 207 | ++ | n.d. |
| 208 | +++ | n.d. |
| 209 | ++ | n.d. |

In Table 2 the results for TB growth inhibition using the colourimetric assay (MABA) and the LORA assay are set out as follows:

TB growth<10 μM=++++; 10-50 μM=+++; 50-128 μM=++; >128 μM but inhibited=+; no inhibition=−; n.d.=not determined.

1. A compound of general formula (I) 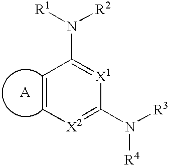
wherein
X¹ is CH or N;
X² is CH or N;
provided that X¹ and X² cannot both be CH;
A is a saturated, unsaturated or partially saturated 5- or 6-membered ring system containing up to three heteroatoms chosen from N, O and S and optionally substituted with one or more substituents selected from halo, OH or C₁-C₆ alkyl, or O—(C₁-C₆ alkyl), either of which may be substituted with one or more halo substituents;
R¹ and R³ are each independently hydrogen or C₁-C₄ alkyl optionally substituted with halo or a group R⁵;
R² and R⁴ are each independently selected from:
(a) a group —C₁-C₆ alkyl- or C₂-C₆ alkenyl, either of which may optionally be substituted with one or more groups NHR⁵, R⁵, R⁶, OR⁶, COR⁶, CO₂R⁶, CONR⁶R⁷;
(b) a group R⁵ or —COR⁵;
each R⁵ is independently an aryl, heteroaryl, carbocyclic or heterocyclic group, any of which may be substituted with one or more substituents chosen from halo, CN, NO₂, R⁶, OR⁶, N(R⁶)₂, COR⁶, CO₂R⁶, SO₂R⁶, (C₁-C₆) alkyl-CO₂R⁶, (C₁-C₆) alkyl-OR⁶, NR⁷COR⁶, NR⁷CO₂R⁶, NR⁷SO₂R⁶, NR⁷CONR⁶R⁷, CONR⁶R⁷, or SO₂NR⁶R⁷;
each R⁶ is independently H, C₁-C₆ alkyl, C₄-C₇ carbocyclyl, C₄-C₇ heterocyclyl or a 5- or 6-membered aromatic or heteroaromatic ring, any of which may be substituted with one or more halo atoms; and
each R⁷ is independently hydrogen or C₁-C₄ alkyl optionally substituted with one or more halo atoms;
or, alternatively, R¹ and R² together with the nitrogen atom to which they are attached may form a 4- to 7-membered heterocyclic ring, optionally containing one or more further heteroatoms chosen from N, O, S, SO or SO₂, optionally fused with a 5- or 6-membered aromatic ring and optionally substituted with one or more substituents selected from R⁵ as defined above or halo, CN, NO₂, R⁶, OR⁶, N(R⁶)₂, COR⁶, CO₂R⁶, SO₂R⁶ and (C₁-C₆) alkyl-CO₂R⁶, (C₁-C₆) alkyl-OR⁶, NR⁷COR⁶, NR⁷CO₂R⁶, NR⁷SO₂R⁶, NR⁷CONR⁶R⁷, CONR⁶R⁷, or SO₂NR⁶R⁷; wherein R⁶ and R⁷ are as defined above;
or, alternatively, R³ and R⁴ together with the nitrogen atom to which they are attached may form a 4- to 7-membered heterocyclic ring, optionally containing one or more further heteroatoms chosen from N, O, S, SO or SO₂, optionally fused with a 5- or 6-membered aromatic ring and optionally substituted with one or more substituents selected from R⁵ as defined above or halo, CN, NO₂R⁶, OR⁶, N(R⁶)₂, COR⁶, CO₂R⁶, SO₂R⁶ and (C₁-C₆) alkyl-CO₂R⁶, (C₁-C₆) alkyl-OR⁶, NR⁷COR⁶, NR⁷CO₂R⁶, NR⁷SO₂R⁶, NR⁷CONR⁶R⁷, CONR⁶R⁷, or SO₂NR⁶R⁷; wherein R⁶ and R⁷ are as defined above;
or a pharmaceutically acceptable salt, hydrate, solvate, complex or prodrug thereof;
for use in the treatment or prevention of a mycobacterial condition.

2. The use of a compound as defined in claim 1 in the preparation of an agent for the treatment or prevention of a mycobacterial condition.

3. A compound of general formula (I) as claimed in claim 1 or the use as claimed in claim 2, wherein the mycobacterial condition is tuberculosis.

4. A compound or the use as claimed in any one of claims 1 to 3 wherein in the compound of general formula (I) A is phenyl.

5. A compound or the use as claimed in any one of claims 1 to 4, wherein in the compound of general formula (I), X¹ is CH and X² is N.

6. A compound or the use as claimed in any one of claims 1 to 4, wherein in the compound of general formula (I), X¹ is N and X² is CH.

7. A compound or the use as claimed in any one of claims 1 to 4, wherein in the compound of general formula (I), both X¹ and X² are N.

8. A compound or the use as claimed in any one of claims 1 to 7, wherein in the compound of general formula (I) R¹ is hydrogen or C₁-C₄ alkyl, optionally substituted with phenyl.

9. A compound or the use as claimed in claim 8 wherein, in the compound of general formula (I), R¹ is hydrogen, methyl or benzyl.

10. A compound or the use as claimed in any one of claims 1 to 9 wherein, in the compound of general formula (I), R² is a carbocyclic moiety; or
a group —C₁-C₄ alkyl-R⁵, where R⁵ is an aryl, heteroaryl, carbocyclyl or heterocyclyl group optionally substituted with halo, CN, NO₂, C₁-C₄ alkyl, C₁-C₄ haloalkyl, O(C₁-C₄ alkyl), N(C₁-C₄ alkyl)₂, CO(C₁-C₄ alkyl), CO₂(C₁-C₄ alkyl) or SO₂(C₁-C₄ alkyl).

11. A compound or the use as claimed in claim 10, wherein in the compound of general formula (I), R² is adamantyl.

12. A compound or the use as claimed in claim 10, wherein in the compound of general formula (I), R² is C₁-C₄ alkyl-R⁵ and R⁵ is phenyl, thiophene, pyridine, naphthalene, indane, cyclohexyl or furyl any of which is optionally substituted with one or more substituents chosen from chloro, fluoro, trifluoromethyl, dimethylamino, methoxy, methyl, ethyl, CO₂CH₃, nitrile and SO₂CH₃.

13. A compound or the use as claimed in any one of claims 1 to 7 wherein, in the compound of general formula (I), R¹ and R² together form a heterocyclic ring system, selected from isoindoline, piperazine, piperidine, tetrahydroisoquinoline, any of which may optionally be substituted with one or more phenyl or halophenyl groups.

14. A compound or the use as claimed in any one of claims 1 to 12 wherein, in the compound of general formula (I), R³ is hydrogen or C₁-C₄ alkyl.

15. A compound or the use as claimed in claim 14 wherein, in the compound of general formula (I), R³ is, hydrogen, methyl or ethyl.

16. A compound or the use as claimed in any one of claims 1 to 14 wherein, in the compound of general formula (I), R⁴ is R⁵, COR⁵ or C₁-C₄ alkyl or C₂-C₄ alkenyl optionally substituted with R⁵, or NHR⁵,
where R⁵ is aryl or heteroaryl optionally substituted as defined in claim 1.

17. A compound or the use as claimed in claim 15 wherein, in the compound of general formula (I), R⁵ is a phenyl group which is optionally substituted with C₁-C₄ alkyl, halo or NO₂.

18. A compound or the use as claimed in any one of claims 1 to 12 wherein, in the compound of general formula (I), R³ and R⁴ together form a heterocyclyl group.

19. A compound or the use as claimed in claim 18 wherein, in the compound of general formula (I), R³ and R⁴ together form a 5- to 7-membered heterocyclic group or a 7-membered ring containing an additional nitrogen or oxygen atom; or such a group fused to a phenyl group, any of which is optionally substituted with CO(C₄-C₇ cycloalkyl), CO-aryl, CO(C₁-C₄ alkyl), CO₂(C₄-C₇ cycloalkyl), CO₂-aryl, CO₂(C₁-C₄ alkyl), SO₂(C₄-C₇ cycloalkyl), SO₂-aryl, SO₂(C₁-C₄ alkyl) or CH₂CO(C₁-C₄ alkyl).

20. A compound or the use as claimed in any one of claims 1 to 19 wherein the compound of general formula (I) is:
N-Benzyl-2-(piperidin-1-yl)quinazolin-4-amine
N-Benzyl-2-morpholinoquinazolin-4-amine
N-Benzyl-2-(4-methylpiperazin-1-yl)quinazolin-4-amine
N¹-(2-Morpholinoquinazolin-4-yl)-N²-(4-nitrophenyl)ethane-1,2-diamine
N-(1-Phenylethyl)-2-(piperazin-1-yl)quinazolin-4-amine
2-Morpholino-N-(1-phenylethyl)quinazolin-4-amine
2-(4-(Methylsulfonyl)piperazin-1-yl)-N-(1-phenylethyl)quinazolin-4-amine
N¹-(2-(4-Methylpiperazin-1-yl)quinazolin-4-yl)-N²-(4-nitrophenyl)ethane-1,2-diamine
N-(1-Phenylethyl)-2-(piperidin-1-yl)quinazolin-4-amine
N-(Furan-2-ylmethyl)-2-(piperidin-1-yl)quinazolin-4-amine
N-(1-Phenylethyl)-2-(pyrrolidin-1-yl)quinazolin-4-amine
N-(1-Phenylethyl)-2-(4-(phenylsulfonyl)piperazin-1-yl)quinazolin-4-amine
N-(2-Adamantyl)-2-morpholinoquinazolin-4-amine
N-Benzyl-6,7-dimethoxy-2-morpholinoquinazolin-4-amine
N²,N⁴-Dibenzylquinazoline-2,4-diamine
N-(1-(2-Adamantyl)ethyl)-2-morpholinoquinazolin-4-amine
N-Benzyl-1-methyl-6-(pyrrolidin-1-yl)-1H-pyrazolo[3,4-d]pyrimidin-4-amine
Cyclopentyl(4-(4-(1-phenylethylamino)quinazolin-2-yl)piperazin-1-yl)methanone
N-Benzyl-2-(pyrrolidin-1-yl)quinazolin-4-amine
N⁴-Benzyl-N²-phenylquinazoline-2,4-diamine
N-Benzyl-4-(piperidin-1-yl)quinazolin-2-amine
N⁴-Benzyl-N²,N²-diethylquinazoline-2,4-diamine
N-Benzyl-2-(4-(ethylsulfonyl)piperazin-1-yl)quinazolin-4-amine
Ethyl 2-(4-(4-(benzylamino)quinazolin-2-yl)piperazin-1-yl)acetate
N⁴-Benzyl-N²-(4-methylbenzyl)quinazoline-2,4-diamine
N²,N⁴-Dibenzyl-N²-methylquinazoline-2,4-diamine
N-Benzyl-2-(3,4-dihydroisoquinolin-2(1H)-yl)quinazolin-4-amine
tert-Butyl 4-(4-(benzylamino)quinazolin-2-yl)piperazine-1-carboxylate
(S)—N-(1-Phenylethyl)-2-(piperidin-1-yl)quinazolin-4-amine
N-Phenethyl-2-(piperidin-1-yl)quinazolin-4-amine
N-(4-Chlorobenzyl)-2-(piperidin-1-yl)quinazolin-4-amine
N-Benzyl-N-methyl-2-(piperidin-1-yl)quinazolin-4-amine
4-(3,4-Dihydroisoquinolin-2(1H)-yl)-2-(piperidin-1-yl)quinazoline
N-(Naphthalen-1-ylmethyl)-2-(piperidin-1-yl)quinazolin-4-amine
2-(Piperidin-1-yl)-N-(4-(trifluoromethyl)benzyl)quinazolin-4-amine
(R)—N-(1-Phenylethyl)-2-(piperidin-1-yl)quinazolin-4-amine
N-(2-Chlorobenzyl)-2-(piperidin-1-yl)quinazolin-4-amine
N-(3-Chlorobenzyl)-2-(piperidin-1-yl)quinazolin-4-amine
2-(Piperidin-1-yl)-N-(2-(trifluoromethyl)benzyl)quinazolin-4-amine
N-(4-Methylbenzyl)-2-(piperidin-1-yl)quinazolin-4-amine
N-(4-Methoxybenzyl)-2-(piperidin-1-yl)quinazolin-4-amine
N-(Cyclohexylmethyl)-2-(piperidin-1-yl)quinazolin-4-amine
2-(Piperidin-1-yl)-N-(pyridin-2-ylmethyl)quinazolin-4-amine
2-(Piperidin-1-yl)-N-(pyridin-3-ylmethyl)quinazolin-4-amine
2-(Piperidin-1-yl)-N-(thiophen-2-ylmethyl)quinazolin-4-amine
2-(Piperidin-1-yl)-N-(3-(trifluoromethyl)benzyl)quinazolin-4-amine
N-Benzhydryl-2-(piperidin-1-yl)quinazolin-4-amine
N-Benzyl-2-(piperazin-1-yl)quinazolin-4-amine
N-Benzyl-2-(1,4-oxazepan-4-yl)quinazolin-4-amine
N-(2-Phenylpropan-2-yl)-2-(piperidin-1-yl)quinazolin-4-amine
N-Benzyl-2-(piperidin-1-yl)quinolin-4-amine
tert-Butyl 4-(4-(benzylamino)quinazolin-2-yl)-1,4-diazepane-1-carboxylate
1-(4-(4-(Benzylamino)quinazolin-2-yl)piperazin-1-yl)-2-methylpropan-1-one
1-(4-(4-(Benzylamino)quinazolin-2-yl)piperazin-1-yl)-2,2-dimethylpropan-1-one
(4-(4-(Benzylamino)quinazolin-2-yl)piperazin-1-yl)(phenyl)methanone
N-(1-Adamantyl)-2-(piperidin-1-yl)quinazolin-4-amine
N-(2-Adamantyl)-2-(piperidin-1-yl)quinazolin-4-amine
N-(1-Adamantyl)-2-morpholinoquinazolin-4-amine
N-(4-(Dimethylamino)benzyl)-2-(piperidin-1-yl)quinazolin-4-amine
N-(4-Fluorobenzyl)-2-(piperidin-1-yl)quinazolin-4-amine
N-(3-Fluorobenzyl)-2-(piperidin-1-yl)quinazolin-4-amine
N-(2-Fluorobenzyl)-2-(piperidin-1-yl)quinazolin-4-amine
N,N-Dibenzyl-2-(piperidin-1-yl)quinazolin-4-amine
N⁴-Benzyl-N²-propylquinazoline-2,4-diamine
N-(3,4-Dichlorobenzyl)-2-(piperidin-1-yl)quinazolin-4-amine
N-(2,4-Dichlorobenzyl)-2-(piperidin-1-yl)quinazolin-4-amine
N-Benzyl-6,7-dimethoxy-2-(piperidin-1-yl)quinazolin-4-amine
N-Benzyl-6-chloro-2-(piperidin-1-yl)quinazolin-4-amine
N-Phenyl-2-(piperidin-1-yl)quinazolin-4-amine
N-(4-(Benzylamino)quinazolin-2-yl)benzamide
N-(4-Chlorobenzyl)-2-morpholinoquinazolin-4-amine
N-(4-Chlorobenzyl)-2-(pyrrolidin-1-yl)quinazolin-4-amine
N⁴-(4-Chlorobenzyl)-N²-(4-methylbenzyl)quinazoline-2,4-diamine
2-Morpholino-N-(4-(trifluoromethyl)benzyl)quinazolin-4-amine
2-(Pyrrolidin-1-yl)-N-(4-(trifluoromethyl)benzyl)quinazolin-4-amine
N²-(4-Methylbenzyl)-N⁴-(4-(trifluoromethyl)benzyl)quinazoline-2,4-diamine
N-(3-Methylbenzyl)-2-(piperidin-1-yl)quinazolin-4-amine
N-(2-Methoxybenzyl)-2-(piperidin-1-yl)quinazolin-4-amine
N-(3-Methoxybenzyl)-2-(piperidin-1-yl)quinazolin-4-amine
N-(4-Chlorophenethyl)-2-(piperidin-1-yl)quinazolin-4-amine
N-(4-Methoxyphenethyl)-2-(piperidin-1-yl)quinazolin-4-amine
4-(Isoindolin-2-yl)-2-(piperidin-1-yl)quinazoline
N-(2,3-Dihydro-1H-inden-2-yl)-2-(piperidin-1-yl)quinazolin-4-amine
N-(4-Methylphenethyl)-2-(piperidin-1-yl)quinazolin-4-amine
4-(4-Phenylpiperazin-1-yl)-2-(piperidin-1-yl)quinazoline
4-(4-(4-Chlorophenyl)piperazin-1-yl)-2-(piperidin-1-yl)quinazoline
4-(4-Phenylpiperidin-1-yl)-2-(piperidin-1-yl)quinazoline
N-(2-Phenylpropan-2-yl)-2-(pyrrolidin-1-yl)quinazolin-4-amine
4-((2-(Piperidin-1-yl)quinazolin-4-ylamino)methyl)benzonitrile
Methyl 4-((2-(piperidin-1-yl)quinazolin-4-ylamino)methyl)benzoate
N-(4-(Methylsulfonyl)benzyl)-2-(piperidin-1-yl)quinazolin-4-amine
N-(3-Phenylpropyl)-2-(piperidin-1-yl)quinazolin-4-amine
(R)—N-(1-(4-Chlorophenyl)ethyl)-2-(piperidin-1-yl)quinazolin-4-amine
(R)—N-(1-(4-Methoxyphenyl)ethyl)-2-(piperidin-1-yl)quinazolin-4-amine
(R)—N-(1-(4-Fluorophenyl)ethyl)-2-(piperidin-1-yl)quinazolin-4-amine
(R)—N-(1-(Naphthalen-1-yl)ethyl)-2-(piperidin-1-yl)quinazolin-4-amine
(R)—N-(1-(Naphthalen-2-yl)ethyl)-2-(piperidin-1-yl)quinazolin-4-amine
(R)-2-(Piperidin-1-yl)-N-(1-p-tolylethyl)quinazolin-4-amine
2-(Piperidin-1-yl)-N-(2-p-tolylpropan-2-yl)quinazolin-4-amine
2-(Pyrrolidin-1-yl)-N-(2-p-tolylpropan-2-yl)quinazolin-4-amine
N-(2-Methylbenzyl)-2-(piperidin-1-yl)quinazolin-4-amine
2-(Piperidin-1-yl)-N-(4-(trifluoromethyl)phenethyl)quinazolin-4-amine
N-(4-Chlorobenzyl)-2-(3,4-dihydroisoquinolin-2(1H)-yl)quinazolin-4-amine
N-Benzyl-5-chloro-2-(piperidin-1-yl)quinazolin-4-amine
N-(1-(4-Fluorophenyl)-2-methylpropan-2-yl)-2-(piperidin-1-yl)quinazolin-4-amine
4-(Piperidin-1-yl)-N-(4-(trifluoromethyl)benzyl)quinazolin-2-amine
N-(4-Chlorobenzyl)-4-(piperidin-1-yl)quinazolin-2-amine
N-(4-Fluorobenzyl)-4-(piperidin-1-yl)quinazolin-2-amine
N-(2,4-Dichlorobenzyl)-4-(piperidin-1-yl)quinazolin-2-amine
2-(3,4-Dihydroisoquinolin-2(1H)-yl)-4-(piperidin-1-yl)quinazoline
2-(3,4-Dihydroisoquinolin-2(1H)-yl)-N-(4-(trifluoromethyl)benzyl)quinazolin-4-amine
N-(2,4-Difluorobenzyl)-2-(piperidin-1-yl)quinazolin-4-amine
N-(3,4-Difluorobenzyl)-2-(piperidin-1-yl)quinazolin-4-amine
(R)-2-(Piperidin-1-yl)-N-(1-(4-(trifluoromethyl)phenyl)ethyl)quinazolin-4-amine
N⁴-(4-Fluorobenzyl)-N²-(4-fluorophenyl)quinazoline-2,4-diamine
N²,N⁴-bis(4-Fluorobenzyl)quinazoline-2,4-diamine
N-(2-(4-Fluorophenyl)propan-2-yl)-2-(piperidin-1-yl)quinazolin-4-amine
N-(2-(4-Fluorophenyl)propan-2-yl)-2-(pyrrolidin-1-yl)quinazolin-4-amine
N-(1-Phenylcyclopropyl)-2-(piperidin-1-yl)quinazolin-4-amine
N-(1-Phenylcyclopropyl)-2-(pyrrolidin-1-yl)quinazolin-4-amine
N-(3-Phenylpentan-3-yl)-2-(pyrrolidin-1-yl)quinazolin-4-amine
N-(1-Phenylcyclohexyl)-2-(pyrrolidin-1-yl)quinazolin-4-amine
N4-Benzyl-N²-(4-fluorobenzyl)quinazoline-2,4-diamine
N4-Benzyl-N²-(4-chlorobenzyl)quinazoline-2,4-diamine
N4-Benzyl-N²-(4-(trifluoromethyl)benzyl)quinazoline-2,4-diamine
N4-Benzyl-N²-(4-methoxybenzyl)quinazoline-2,4-diamine
N-Benzyl-2-(isoindolin-2-yl)quinazolin-4-amine
N4-Benzyl-N²-(2,3-dihydro-1H-inden-2-yl)quinazoline-2,4-diamine
N-Benzyl-2-(4-phenylpiperidin-1-yl)quinazolin-4-amine
N-Benzyl-2-(4-phenylpiperazin-1-yl)quinazolin-4-amine
N-Benzyl-2-(4-(4-chlorophenyl)piperazin-1-yl)quinazolin-4-amine
N-Benzyl-2-(4-(4-(trifluoromethyl)phenyl)piperazin-1-yl)quinazolin-4-amine
N-Benzyl-2-(4-(4-methoxyphenyl)piperazin-1-yl)quinazolin-4-amine
2-(4,4-Difluoropiperidin-1-yl)-N-(4-fluorobenzyl)quinazolin-4-amine
N-(3-Phenylpentan-3-yl)-2-(piperidin-1-yl)quinazolin-4-amine
1-(4-(Benzylamino)quinazolin-2-yl)piperidin-4-one
N-Benzyl-2-thiomorpholinoquinazolin-4-amine
N-(1-(4-Chlorophenyl)cyclopropyl)-2-(piperidin-1-yl)quinazolin-4-amine
N-(1-(4-Chlorophenyl)cyclopropyl)-2-(pyrrolidin-1-yl)quinazolin-4-amine
(S)-3-Phenyl-2-(2-(piperidin-1-yl)quinazolin-4-ylamino)propan-1-ol
(S)—N-(1-Methoxy-3-phenylpropan-2-yl)-2-(piperidin-1-yl)quinazolin-4-amine
(R)-2-Phenyl-2-(2-(piperidin-1-yl)quinazolin-4-ylamino)ethanol
N-(4-Chlorobenzyl)-4-(4,4-difluoropiperidin-1-yl)quinazolin-2-amine
4-(4,4-Difluoropiperidin-1-yl)-N-(4-(trifluoromethyl)benzyl)quinazolin-2-amine
N-(2,4-Difluorobenzyl)-4-(4,4-difluoropiperidin-1-yl)quinazolin-2-amine
4-(4,4-Difluoropiperidin-1-yl)-N-(4-fluorobenzyl)quinazolin-2-amine
N-(4-Fluorobenzyl)-6,7-dimethoxy-2-(piperidin-1-yl)quinazolin-4-amine
2-(4,4-Difluoropiperidin-1-yl)-N-(4-fluorobenzyl)-6,7-dimethoxyquinazolin-4-amine
N-(1-(4-Fluorophenyl)cyclopropyl)-2-(piperidin-1-yl)quinazolin-4-amine
2-(4,4-Difluoropiperidin-1-yl)-N-(1-(4-fluorophenyl)cyclopropyl)quinazolin-4-amine
N²-(4-Fluorobenzyl)-N⁴-(1-(4-fluorophenyl)cyclopropyl)quinazoline-2,4-diamine
7-Fluoro-N-(4-fluorobenzyl)-2-(piperidin-1-yl)quinazolin-4-amine
2-(4,4-Difluoropiperidin-1-yl)-7-fluoro-N-(4-fluorobenzyl)quinazolin-4-amine
7-Fluoro-N²,N⁴-bis(4-fluorobenzyl)quinazoline-2,4-diamine
N²-(4-Fluorobenzyl)-N⁴-((5-methylfuran-2-yl)methyl)quinazoline-2,4-diamine
N²-(4-Fluorobenzyl)-N⁴4(5-(trifluoromethyl)furan-2-yl)methyl)quinazoline-2,4-diamine
2-(Piperidin-1-yl)-N-((5-(trifluoromethyl)furan-2-yl)methyl)quinazolin-4-amine
2-(4,4-Difluoropiperidin-1-yl)-N-((5-(trifluoromethyl)furan-2-yl)methyl)quinazolin-4-amine
2-(Piperidin-1-yl)-N-(4-(trifluoromethoxy)benzyl)quinazolin-4-amine
2-(4,4-Difluoropiperidin-1-yl)-N-(4-(trifluoromethoxy)benzyl)quinazolin-4-amine
4-((2-(Piperidin-1-yl)quinazolin-4-ylamino)methyl)phenol
4-((2-(4-Fluorobenzylamino)quinazolin-4-ylamino)methyl)phenol
N², N⁴-bis(4-Fluorobenzyl)thieno[3,2-d]pyrimidine-2,4-diamine
N-((5-Methylfuran-2-yl)methyl)-2-(piperidin-1-yl)quinazolin-4-amine
N²,N⁴-bis(4-Fluorobenzyl)-6,7-dimethoxyquinazoline-2,4-diamine
N-(4-Fluorobenzyl)-2-morpholinoquinazolin-4-amine
N⁴-(4-Fluorobenzyl)-N²-((5-methylfuran-2-yl)methyl)quinazoline-2,4-diamine
N²-(Benzo[b]thiophen-2-ylmethyl)-N⁴-(4-fluorobenzyl)quinazoline-2,4-diamine
N-(4-Fluorobenzyl)-2-(pyrrolidin-1-yl)quinazolin-4-amine
N-(4-Fluorobenzyl)-2-(pyrrolidin-1-yl)pyrido[2,3-d]pyrimidin-4-amine
N-(4-Fluorobenzyl)-2-(piperidin-1-yl)pyrido[2,3-d]pyrimidin-4-amine
(S)-Methyl 2-phenyl-2-(2-(piperidin-1-yl)quinazolin-4-ylamino)acetate
(S)-Methyl 3-phenyl-2-(2-(piperidin-1-yl)quinazolin-4-ylamino)propanoate
(S)-(1-(4-(Benzylamino)quinazolin-2-yl)pyrrolidin-2-yl)methanol
(S)—N-Benzyl-2-(2-(methoxymethyl)pyrrolidin-1-yl)quinazolin-4-amine
N-(4-Fluorobenzyl)-2-(isoindolin-2-yl)quinazolin-4-amine
(S)-Methyl 1-(4-(benzylamino)quinazolin-2-yl)pyrrolidine-2-carboxylate
N-Benzyl-2-(6,7-dimethoxy-3,4-dihydroisoquinolin-2(1H)-yl)quinazolin-4-amine
5-Chloro-N-(4-fluorobenzyl)-2-(pyrrolidin-1-yl)quinazolin-4-amine
5-Chloro-N-(4-fluorobenzyl)-2-(piperidin-1-yl)quinazolin-4-amine
tert-Butyl-4-(4-fluorobenzylamino)-2-(piperidin-1-yl)-5,6-dihydropyrido-[3,4-d]pyrimidine-7(8H)-carboxylate
N-(4-Fluorophenyl)-2-(pyrrolidin-1-yl)pteridin-4-amine
4-(3,4-Dihydroisoquinolin-2(1H)-yl)-2-(pyrrolidin-1-yl)quinazoline
2-(Pyrrolidin-1-yl)-N-(thiophen-2-ylmethyl)quinazolin-4-amine
4-(6,7-Dimethoxy-3,4-dihydroisoquinolin-2(1H)-yl)-2-(pyrrolidin-1-yl)quinazoline
N-((5-Chlorothiophen-2-yl)methyl)-2-(piperidin-1-yl)quinazolin-4-amine
N-(Furan-3-ylmethyl)-2-(piperidin-1-yl)quinazolin-4-amine
2-(Piperidin-1-yl)-N-(thiophen-3-ylmethyl)quinazolin-4-amine
4-(6,7-Dimethoxy-3,4-dihydroisoquinolin-2(1H)-yl)-2-(piperidin-1-yl)quinazoline
2-(7-Bromo-3,4-dihydroisoquinolin-2(1H)-yl)-N-(4-fluorobenzyl)quinazolin-4-amine
N-((6-Chloropyridin-3-yl)methyl)-2-(piperidin-1-yl)quinazolin-4-amine
2-(Piperidin-1-yl)-N-(quinolin-4-ylmethyl)quinazolin-4-amine
2-(Piperidin-1-yl)-N-((6-(trifluoromethyl)pyridin-3-yl)methyl)quinazolin-4-amine
2-(Azepan-1-yl)-N-benzylquinazolin-4-amine
(S)-(1-(4-(4-Fluorobenzylamino)quinazolin-2-yl)pyrrolidin-2-yl)methanol
(S)—N-(4-Fluorobenzyl)-2-(2-(methoxymethyl)pyrrolidin-1-yl)quinazolin-4-amine
(S)-Methyl 1-(4-(4-fluorobenzylamino)quinazolin-2-yl)pyrrolidine-2-carboxylate
2-(4-Benzylpiperazin-1-yl)-N-(4-fluorobenzyl)quinazolin-4-amine
Cyclopropyl(4-(4-(4-fluorobenzylamino)quinazolin-2-yl)piperazin-1-yl)methanone
Cyclohexyl(4-(4-(4-fluorobenzylamino)quinazolin-2-yl)piperazin-1-yl)methanone
(4-(4-(4-Fluorobenzylamino)quinazolin-2-yl)piperazin-1-yl)(phenyl)methanone
(4-(4-(4-Fluorobenzylamino)quinazolin-2-yl)piperazin-1-yl)(pyrrolidin-1-yl)methanone
Ethyl 2-(4-(4-fluorobenzylamino)quinazolin-2-ylamino)acetate
N⁴-(4-Fluorobenzyl)-N²-(2-methoxyethyl)quinazoline-2,4-diamine
N²-(Cyclohexylmethyl)-N⁴-(4-fluorobenzyl)quinazoline-2,4-diamine
N⁴-(4-Fluorobenzyl)-N²-(2-(piperidin-1-yl)ethyl)quinazoline-2,4-diamine
or a pharmaceutically acceptable salt hydrate solvate, complex or prodrug thereof.

21. The use as claimed in any one of claims 2 to 20, wherein the antibacterial agent also contains one or more additional compounds useful for the treatment of TB.

22. A pharmaceutical composition comprising a compound of general formula (I) as defined in any one of claims 1 to 20 together with one or more additional compounds useful in the treatment of TB and a pharmaceutically acceptable excipient.

23. A product comprising a compound of general formula (I) as defined in any one of claims 1 to 20 and one or more compounds useful in the treatment of TB as a combined preparation for simultaneous, separate or sequential use in the treatment of tuberculosis.

24. The use, composition or product as claimed in any one of claims 21 to 23 wherein the one or more compounds useful in the treatment of TB is selected from isoniazid, rifamycin and derivatives thereof, pyrazinamide, ethambutol, cycloserine, ethionamide, streptomycin, amikacin, kanamycin, capreomycin, p-aminosalicylic acid, and fluoroquinolones such as levofloxacin, moxafloxacin or gatifloxacin.
